(19) United States
(12) Patent Application Publication
(10) Pub. No.: US 2012/0115207 A1
(43) Pub. Date: May 10, 2012

(54) ENHANCED PRODUCTION OF PAPILLOMAVIRUS-LIKE PARTICLES WITH A MODIFIED BACULOVIRUS EXPRESSION SYSTEM

(75) Inventors: Tilo Senger, Heidelberg (DE); Martin Muller, Neckargemund (DE); Lutz Gissmann, Wiesloch (DE)

(73) Assignee: Deutsches Krebsforschungszentrum

(21) Appl. No.: 13/257,248

(22) PCT Filed: Mar. 30, 2010
(86) PCT No.: PCT/EP2010/054258

§ 371 Date: Dec. 5, 2011

(30) Foreign Application Priority Data
Apr. 3, 2009 (EP) 09157278.4

(51) Int. Cl. C12N 7/02; C12N 15/85; C12N 5/10

(52) U.S. Cl. 435/239; 435/348; 435/320.1

(57) ABSTRACT

The present invention is concerned with the provision of a method for manufacturing papillomavirus like particles (PV-VLP), comprising the steps of a) culturing a host cell lacking protease activity and comprising an expression vector, wherein said expression vector comprises at least one polynucleotide encoding a PV L1 polypeptide, and b) obtaining VLPs from the host cell. Also proposed is a host cell lacking protease activity and comprising an expression vector, wherein said expression vector comprises a polynucleotide encoding at least one PV L1 polypeptide. Furthermore, a method for the manufacture of a pharmaceutical composition for the treatment or prevention of PV-related disease comprising the steps of manufacturing PV-VLPs and the further step of formulating the VLPs as a pharmaceutical composition is proposed as well as an expression vector comprising at least one polynucleotide encoding a PV L1 polypeptide but lacking a functional gene for a v-cath protease.

Papillomaviruses (PV) are a group of small, non-enveloped dsDNA-viruses that infect skin and mucous membranes of a variety of animal species, causing the formation of benign epithelial tumors, or warts, at the infection site. Usually, a distinct group of PV has the ability to infect a certain vertebrate species, where each of these groups comprises several PV types.

In humans, more than 100 different types of human papillomaviruses (HPV) have been characterized, some of which cause genital warts, which are a usually benign sexually transmitted disease. In some cases, however, HPV lesions can enter malignant progression and may eventually lead to cancer. This progression can occur after infection with one of the so-called “high-risk” types of HPV. Sexually transmitted, high-risk HPV types include types 16, 18, 31, 33, 35, 39, 45, 51, 52, 56, 58, 59, 66, 68, and 73 (Munoz N et al. (2004), Against which human papillomavirus types shall we vaccinate and screen? The international perspective. Int J Cancer 111:278-285).

Consequently, vaccination schedules have been devised to protect women from infection with high-risk HPV types. Gardasil, which is marketed by Merck, contains the structural L1-proteins from HPV types -16, -18, -6, and -11, of which HPV-16 and -18 are estimated to be responsible for approximately 70% of cervix cancer cases. In a parallel development, GlaxoSmithKline introduced Cervarix, which is a vaccine containing L1-proteins of HPV-16 and -18. The inclusion of several types of HPV in vaccines is necessitated by the fact that HPV vaccination usually is type-specific, conferring little protection even against closely related types. Hence, it would be desirable to include more HPV types in vaccine formulations in order to extend protection to more rare, but nonetheless high-risk, HPV types.

The HPV virus-like-particles (VLP) used as vaccines are produced using recombinant DNA technology in baker's yeast (Gardasil) or in an insect cell system (Cervarix). Production of VLPs in insect cell systems was found to give satisfactory yield for some HPV types, but failed to produce detectable amounts of VLPs for others.

Thus, the technical problem underlying the present invention may be seen as the provision of means and methods for the manufacturing of PV L1-VLPs with high yield. The technical problem is solved by the embodiments characterized in the claims and herein below.

Accordingly, the present invention relates to a method for manufacturing papillomavirus like particles (PV-VLP), comprising the steps of a) culturing a host cell lacking protease activity and comprising an expression vector, wherein said expression vector comprises at least one polynucleotide encoding a PV L1 polypeptide, and b) obtaining VLPs from the host cell.

The method of the present invention, preferably, may comprise steps in addition to those explicitly mentioned above. For example, further steps may relate to providing a suitable number of cells to be used as host cells or separating VLPs from other proteins in the reaction mixture. The method may be carried out manually or assisted by automation. Preferably, step (a) and/or (b) may in total or in part be assisted by automation.

The term “Papillomavirus” (PV) as used herein relates to viruses from the family Papillomaviridae. Non-limiting examples of PV groups are Human papillomaviruses (HPV), i.e. papillomaviruses that infect man,

and bovine papillomaviruses (BPV), i.e. papillomaviruses that infect cattle or horses. “PV type” relates to a subgroup of PV distinguished on the basis of sequence relatedness.

The term “Papillomavirus like particles” (PV-VLPs) as used herein relates to protein aggregates comprising the L1 major capsid protein of PV. Preferably, the proportion of PV L1 polypeptide in said aggregates is at least 50%, 70%, 80%, 90%, 95%, 99%. In another preferred embodiment, PV-VLPs comprise modified forms of the PV L1 polypeptide as described below. Furthermore, it is also contemplated by the present invention that PV-VLPs comprise L1 polypeptides from more than one type of PV.

The term “host cell”, preferably, relates to a cell maintained in vitro in a suitable cultivation medium and capable of producing PV L1 polypeptides. Preferably, said cell is a eukaryotic cell, more preferably an insect cell, still more preferably a lepidopteran cell, and most preferably a cell selected from the group consisting of Sf9, Sf21, Express SF+, and BTITn-5B1-4 (“TN High Five”).

The term “culturing a host cell” as used herein relates to incubating a host cell comprising the properties as specified below under conditions suitable for the production of VLPs. Preferably, said conditions are the conditions suited optimally for the growth of the respective host cell, which vary with the type of host cell and which are well known in the art (see, for example, Example 1). It is, however, also contemplated by the current invention that host cells are cultured by transferring host cells into a suitable animal, e.g. the abdominal cavity of a rodent. In another preferred embodiment, host cells are cells comprised in larvae of lepidopterans.

The term “lacking protease activity” as used herein relates to the absence of enzymatic activity hydrolysing a PV L1 polypeptide in host cells and/or in the cultivation medium. Preferably, said protease is a member of the cathepsin family of proteases and most preferably, said protease is a viral-cathepsin (v-cath; non-limiting examples are Genbank Accession number: M67451.1 (GI:332490), v-cath from Autographa californica nucleopolyhedrovirus; Genbank Accession number: NP_{—}203280.1 (GI:15320768), v-cath from Epiphyas postvittana nucleopolyhedrosis virus; and Genbank Accession number: YP_{—}717598.1 (GI:113195461), v-cath from Clanis bilineata nucleopolyhedrosis virus). Methods to achieve the absence of protease activity are well known in the art, addition of one or more protease inhibitor(s) being the most prevalent one. Specific inhibitors for members of the cathepsin family of proteases may be, but are not limited to, Hippuryl-Arginine for Cathepsin B, Gly-Phe p-nitroanilide for Cathepsin C, N-Acetyl-Arg-Gly-Phe-Phe-Pro 4-methoxy-2-naphthylamide for Cathepsin D, N-Methoxysuccinyl-Ala-Ala-Pro-Met p-nitroanilide for Cathepsin G, and Z-Phe-Arg 4-methoxy-naphthylamide for Cathepsin L.

It is, however, also contemplated by the present invention, that the lack of protease activity is achieved by the absence of an expressible gene for a v-cath protease, said gene being comprised in most baculovirus vectors. Methods to modify vectors to remove functional genes are well known in the art (see also Example 2 and references therein). Preferably, the absence of v-cath activity is achieved by modification of the regulatory sequence of the v-cath gene in a way that abolishes expression of said gene, e.g. by the exchange and/or deletion and/or insertion of one or more nucleotides; in another preferred embodiment, the open reading frame of the v-cath gene is modified in a way that abolishes expression of the gene and/or the proteolytic activity of the v-cath protease. Preferably, said modification of the open reading frame is an insertion and/or deletion and/or exchange of one or more nucleotides, leading e.g. to the introduction of a premature stop codon, a frameshift mutation, or the deletion of a part of or the complete open reading frame. More preferably, said modification is the modification comprised in the MultiBac vector as described in WO2005/085456. In another preferred embodiment, absence or reduction of v-cath activity is achieved by the absence of an expressible gene for chiA, said gene coding for an activator of the v-cath protease (Hom, L. G., and Volkman, L. E. (2000). Autographa californica M nucleopolyhedrovirus chiA is required for processing of V-CATH. Virology 277(1), 178-83). Absence of an expressible gene for chiA can be achieved by the same methods as detailed above for v-cath. Most preferably, cells lacking protease activity are obtained by the simultaneous inactivation of the genes for chiA and for v-cath.

The term “vector”, preferably, encompasses phage, plasmid, or viral vectors as well as artificial chromosomes, such as bacterial or yeast artificial chromosomes. The vector encompassing the polynucleotides of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Viral vectors may be replication competent or replication defective. In the latter case, viral propagation generally will occur only in complementing host cells.

The term “expression vector” as used herein relates to a vector comprising at least one, two, three, four polynucleotides encoding a PV L1 polypeptide as described below operatively linked to expression control sequences allowing expression in host cells or in isolated fractions thereof. Expression of said polynucleotide comprises transcription of the polynucleotide, preferably into a translatable mRNA. Regulatory elements ensuring expression in eukaryotic cells, preferably insect cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in host cells comprise, e.g., the CMV-, SV40-, RSV-promoter (Rous sarcoma virus), CMV-enhancer, SV40-enhancer or a globin intron in mammalian and other animal cells, and polh and P10 Promoters in insect cell systems. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription, such regulatory elements may also comprise transcription termination signals, such as the SV40-poly-A site or the tk-poly-A site, downstream of the polynucleotide. In this context, suitable expression vectors are known in the art such as MultiBac (WO 2005/085456), pFastBac™DUAL (Invitrogen), and bMON14272. Expression vectors derived from viruses such as baculovirus may be used for delivery of the polynucleotides or vector of the invention into host cells. Methods which are well known to those skilled in the art can be used to construct recombinant viral vectors; see, for example, the techniques described in Sambrook, Molecular Cloning: A Laboratory Manual, Cold Spring Harbor Laboratory (1989) N.Y. and Ausubel, Current Protocols in Molecular Biology, Green Publishing Associates and Wiley Interscience, N.Y. (1994) and in Example 2. Furthermore, it is contemplated by the current invention that the expression vector contains at least one, at least two, at least three, at least four different polynucleotide(s) that are comprised in at least one, at least two, at least three, at least four different PV L1 polypeptides.

The term “polynucleotide” as used in accordance with the present invention relates to a polynucleotide comprising a nucleic acid sequence which encodes an L1 major capsid protein comprised in a PV, said L1 polypeptide having the biological activity of forming VLPs. Examples of suitable assays for detecting the L1 polypeptide and the activity of the L1 polypeptide to form VLPs are described in the accompanying examples. Thus, the polynucleotide, preferably, comprises a nucleic acid sequence coding for an L1 protein selected from the list consisting of gene_ID 1489082 in GENBANK ACC No: NC_{—}001526.1 GI:9627100 (complete genome of Human papillomavirus type 16, representing the alpha genus of papillomaviruses), gene_ID 1489054 in GENBANK ACC No: NC_{—}001531.1 GI:9627145 (complete genome of Human papillomavirus type 5, representing the beta genus of papillomaviruses), gene_ID 1488986 in GENBANK ACC No: NC_{—}001523.1 GI:9627065 (complete genome of Deer papillomavirus, representing the delta genus of papillomaviruses), gene_ID 955406 in GENBANK ACC No: NC_{—}004195.1 GI:23217014 (complete genome of Bovine papillomavirus type 5, representing the epsilon genus of papillomaviruses), gene_ID 1489455 in GENBANK ACC No: NC_{—}001457.1 GI:9626597 (complete genome of Human papillomavirus type 4, representing the gamma genus of papillomaviruses), gene_ID 1489003 in GENBANK ACC No: NC_{—}001605.1 GI:9627486 (complete genome of Multimammate rat papillomavirus, representing the iota genus of papillomaviruses), gene_ID 1460791 in GENBANK ACC No: NC_{—}002232.1 GI:9635132 (complete genome of Rabbit oral papillomavirus, representing the kappa genus of papillomaviruses), gene_ID 1497245 in GENBANK ACC No: NC_{—}001619.1 GI:9627734 (complete genome of Canine oral papillomavirus, representing the lambda genus of papillomaviruses), gene_ID 1494575 in GENBANK ACC No: NC_{—}001458.1 GI:9626605 (complete genome of Human papillomavirus type 63, representing the mu genus of papillomaviruses), gene_ID 1489283 in GENBANK ACC No: NC_{—}001354.1 GI:9626041 (complete genome of Human papillomavirus type 41, representing the nu genus of papillomaviruses), gene_ID 929650 in GENBANK ACC No: NC_{—}003348.1 GI:18138516 (complete genome of Phocoena spinipinnis papillomavirus, representing the omikron genus of papillomaviruses), gene_ID 944558 in GENBANK ACC No: NC_{—}003973.1 GI:21326229 (complete genome of Psittacus erithacus timneh papillomavirus, representing the theta genus of papillomaviruses), gene_ID 5845995 in GENBANK ACC No: NC_{—}010192.1 GI:164398797 (complete genome of Bovine papillomavirus-9, representing the Xipa genus of papillomaviruses), and gene_ID 944325 in GENBANK ACC No: NC_{—}003748.1 GI:20428628 (complete genome of Equus caballus papillomavirus-1, representing the zeta genus of papillomaviruses). It is to be understood that a polypeptide having an amino acid sequence as selected from a list consisting of GENBANK ACC No: NP_{—}041332.1 GI:9627108 (L1 protein of Human papillomavirus type 16), GENBANK ACC No: NP_{—}041372.1 GI:9627153 (L1 protein of Human papillomavirus type 5) GENBANK ACC No: NP_{—}041300.1 GI:9627073 (L1 protein of Deer papillomavirus), GENBANK ACC No: NP_{—}694435.1 GI:23217020 (L1 protein of Bovine papillomavirus type 5), GENBANK ACC No: NP_{—}040895.1 GI:9626604 (L1 protein of Human papillomavirus type 4), GENBANK ACC No: NP_{—}042019.1 GI:9627492 (L1 protein of Multimammate rat papillomavirus), GENBANK ACC No: NP_{—}057848.1 GI:9635141 (L1 protein of Rabbit oral papillomavirus), GENBANK ACC No: NP_{—}056819.1 GI:9627741 (L1 protein of Canine oral papillomavirus), GENBANK ACC No: NP_{—}040902.1 GI:9626612 (L1 protein of Human papillomavirus type 63), GENBANK ACC No: NP_{—}040294.1 GI:9626049 (L1 protein of Human papillomavirus type 41), GENBANK ACC No: NP_{—}542623.1 GI:18138524 (L1 protein of Phocoena spinipinnis papillomavirus), GENBANK ACC No: NP_{—}647590.1 GI:21326235 (L1 protein of Psittacus erithacus timneh papillomavirus), GENBANK ACC No: YP_{—}001648349.1 GI:164398803 (L1 protein of Bovine papillomavirus-9), and GENBANK ACC No: NP_{—}620513.1 GI:20428635 (L1 protein of Equus caballus papillomavirus-1) may be also encoded due to the degenerated genetic code by other polynucleotides as well.

Moreover, also encompassed are polynucleotides which comprise nucleic acid sequences encoding amino acid sequences which are at least 50%, at least 60%, at least 70%, at least 75%, at least 80%, at least 85%, at least 90%, at least 95%, at least 98% or at least 99% identical to the amino acid sequences above. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software packet [Genetics Computer Group, 575 Science Drive, Madison, Wis., USA 53711 (1991)], are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000.

Moreover, the term “polynucleotide” as used in accordance with the present invention further encompasses variants of the aforementioned specific polynucleotides. Said variants may represent orthologs, paralogs or other homologs of the polynucleotide of the present invention. The polynucleotide variants, preferably, comprise a nucleic acid sequence characterized in that the sequence can be derived from the aforementioned specific nucleic acid sequences described above by at least one nucleotide substitution and/or addition and/or deletion whereby the variant nucleic acid sequence shall still encode a polypeptide having the ability to form VLPs. The ability of a polypeptide to form VLPs can be monitored e.g. by centrifugation through a sucrose gradient, by an equilibrium centrifugation in a CsCl density gradient, or by transmission electron microscopy as described in Examples 3 to 6. Moreover, in a preferred embodiment, a polynucleotide comprises a nucleic acid sequence encoding an L1 polypeptide of a PV selected from the group consisting of HPV-2 (GENBANK ACC No: NP_{—}077122.1 GI:13186282, HPV-3 (GENBANK ACC No: CAA52475.1 GI:397013), HPV-10 (GENBANK ACC No: NP_{—}041747.1 GI:9627264), HPV-27 (GENBANK ACC No: CAA52542.1 GI:396972), HPV-57 (GENBANK ACC No: CAA39436.1 GI:60889), HPV-77 (GENBANK ACC No: CAA75468.1 GI:2911564), bovine papillomavirus (BPV)-5 (GENBANK ACC No: NP_{—}694435.1 GI:23217020), and BPV-6 (GENBANK ACC No: CAF05691.1 GI:40804508).

A polynucleotide comprising a fragment of any of the aforementioned nucleic acid sequences is also encompassed as a polynucleotide of the present invention. The fragment shall encode a polypeptide which still has the activity as specified above. Accordingly, the polypeptide may comprise or consist of the domains of the L1 polypeptide of the present invention conferring the activity of forming VLPs. A fragment as meant herein, preferably, comprises at least 50, at least 100, at least 250 or at least 500, at least 750, at least 1000 or at least 1500 consecutive nucleotides of any one of the aforementioned nucleic acid sequences or encodes an amino acid sequence comprising at least 20, at least 30, at least 50, at least 80, at least 100, at least 150, at least 200, at least 250 or at least 300 consecutive amino acids of any one of the aforementioned amino acid sequences.

The polynucleotides of the present invention either essentially consist of the aforementioned nucleic acid sequences or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part other PV polypeptides, polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, 6-histidine-tags, MYC-tags and the like.

The polynucleotide of the present invention shall be provided, preferably, either as an isolated polynucleotide (i.e. isolated from its natural context) or in genetically modified form. The polynucleotide, preferably, is RNA or DNA, including cDNA. The term encompasses single as well as double stranded polynucleotides. Moreover, comprised are also chemically modified polynucleotides including naturally occurring modified polynucleotides such as glycosylated or methylated polynucleotides or artificial modified ones such as biotinylated polynucleotides.

The term “obtaining VLPs” as used herein, preferably, relates to separating VLPs from host cells in a way that makes VLPs amenable for further use. Methods used for obtaining VLPs are well known in the art. The choice of methods depends on the purity required and the further use intended. For example, VLPs may be obtained by centrifugation or by equilibrium centrifugation in CsCl density gradients or by affinity chromatography or by heparin affinity chromatography (see Example 3). Other methods for obtaining VLPs include but are not limited to anion exchange chromatography, hydroxyapatite chromatography, or size exclusion chromatography, alone or in combination.

In another preferred embodiment, the present invention relates to a host cell lacking protease activity and comprising an expression vector, wherein said expression vector comprises a polynucleotide encoding at least one PV L1 polypeptide as specified above.

In a further preferred embodiment, the present invention relates to a method for the manufacture of a pharmaceutical composition for the treatment or prevention of PV-related disease comprising the steps of the method as detailed above and the further step of formulating the VLPs as a pharmaceutical composition.

The term “pharmaceutical composition” as used herein comprises the compounds of the present invention and optionally one or more pharmaceutically acceptable carrier. The compounds of the present invention can be formulated as pharmaceutically acceptable salts. Acceptable salts comprise acetate, methylester, HCl, sulfate, chloride and the like. The pharmaceutical compositions are, preferably, administered topically or systemically. Suitable routes of administration conventionally used for drug administration are oral, intravenous, or parenteral administration as well as inhalation. However, depending on the nature and mode of action of a compound, the pharmaceutical compositions may be administered by other routes as well. For example, polynucleotide compounds may be administered by using viral vectors or viruses or liposomes.

Moreover, the compounds can be administered in combination with other drugs either in a common pharmaceutical composition or as separated pharmaceutical compositions wherein said separated pharmaceutical compositions may be provided in form of a kit of parts.

The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of 0.1 to 1 μg/kg body mass; however, doses below or above this exemplary range are envisioned, especially considering the aforementioned factors.

The pharmaceutical compositions and formulations referred to herein are administered at least once in order to treat or ameliorate or prevent a disease or condition recited in this specification. However, the said pharmaceutical compositions may be administered more than one time, for example from once per week for up to one year.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers' or users' instructions in order to anticipate dose adjustments depending on the considered recipient.

The term “treatment” refers to amelioration of the diseases or disorders referred to herein or the symptoms accompanied therewith to a significant extent. Said treatment as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

The term “prevention” refers to preservation of health with respect to the diseases or disorders referred to herein for a certain period of time in a subject. It will be understood that the said period of time is dependent on the amount of the drug compound which has been administered and individual factors of the subject discussed elsewhere in this specification. It is to be understood that prevention may not be effective in all subjects treated with the compound according to the present invention. However, the term requires that a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “PV-related disease” as used herein relates to a temporary or persistent impairment of health related to PV infection. Preferably, PV-related disease is the development of epithelial tumors, or warts, on and/or in the skin and/or mucous membranes. Epithelial tumors caused by PV are, exemplarily, common warts (verrucae) of the skin, plantar warts on the soles of the feet, warts on the larynx and/or other parts of the respiratory tract (respiratory papillomatosis), and genital and anal warts. More preferably, PV-related disease is the occurrence of cancer after infection of an individual with PV. Examples of precancerous and cancerous lesions associated with HPV infection are, but are not limited to, Cervical Intraepithelial Neoplasia (CIN), cervical cancer, anal cancer, vaginal and/or vulvar cancer, penile cancer (Parkin D M (2006). “The global health burden of infection-associated cancers in the year 2002”. Int. J. Cancer 118 (12): 3030-3044), and oropharyngeal squamous-cell carcinoma (D'Souza G, Kreimer A R, Viscidi R, et al (2007). “Case-control study of human papillomavirus and oropharyngeal cancer”. N. Engl. J. Med. 356 (19): 1944-1956). It is also contemplated by the current invention that PV-related disease is a disease of an animal, preferably a mammal, said disease being related to PV-infection. Examples of PV-related disease in animals are warts in cattle or sarcoid in horses.

In a final embodiment, the present invention relates to an expression vector comprising at least one polynucleotide encoding a PV L1 polypeptide and lacking a functional gene for a v-cath protease and/or for chiA, the term “lacking a functional gene for a v-cath protease and/or for chiA” relating to the absence of an expressible gene for a v-cath protease and/or for chiA as specified above.

TABLE 1
MultiBac expression system permits efficient VLP production of various
PV constructs and it enables production of mutant HPV constructs.

| | | Conventional | | MultiBac | | | | | |
|---|---|---|---|---|---|---|---|---|---|
| | | polh | | polh | | p10 | | polh/p10 | |
| WT L1 proteins | HPV2 | ≦0.05 | mg | 0.2-0.4 | mg | 0.1-0.2 | mg | 0.8-1.5 | mg |
| | HPV3 | 0.1 | mg | N/A | | N/A | | 2.0 | mg |
| | HPV6* | <det | | N/A | | N/A | | 0.7 | mg |
| | HPV10 | 0.1 | mg | N/A | | N/A | | 4.0 | mg |
| | HPV11* | ≦0.15 | mg | N/A | | N/A | | 1.2 | mg |
| | HPV18* | ≦0.15 | mg | N/A | | 0.9 | mg | 1.3 | mg |
| | HPV27 | ≦0.05 | mg | 0.15-0.4 | mg | 0.1-0.3 | mg | 0.8-1.2 | mg |
| | HPV57 | ≦0.15 | mg | 0.6-1.2 | mg | 0.3-0.8 | mg | 1.5-2.8 | mg |
| | HPV77 | 0.5 | mg | N/A | | N/A | | 4.0 | mg |
| | BPV5 | <det | | N/A | | 1.2 | mg | 2.5 | mg |
| | BPV6 | <det | | N/A | | 1.2 | mg | 3.2 | mg |
| Mutant L1 proteins | HPV2 C172S, C422S L1 | ≦0.05 | mg | 0.1-0.2 | mg | N/A | | 0.3-0.5 | mg |
| | HPV27 C173S, C424S L1 | ≦0.05 | mg | 0.1-0.15 | mg | N/A | | 0.2-0.4 | mg |
| | HPV57 C173S, C4283S L1 | ≦0.05 | mg | 0.1-0.2 | mg | N/A | | 0.2-0.35 | mg |
| | HPV16 L1E7(1-60*) | ≦0.05 | mg | 3.4 | mg | 5.1 | mg | 4.3 | mg |

The yield for the different baculovirus stocks infecting 10⁸ High Five insect cells at an MOI of 2 is shown.
<det: below detection limit;
N/A: not analyzed
*for these types MultiBac-based viruses were generated with codon-modified L1 genes

The figures show:

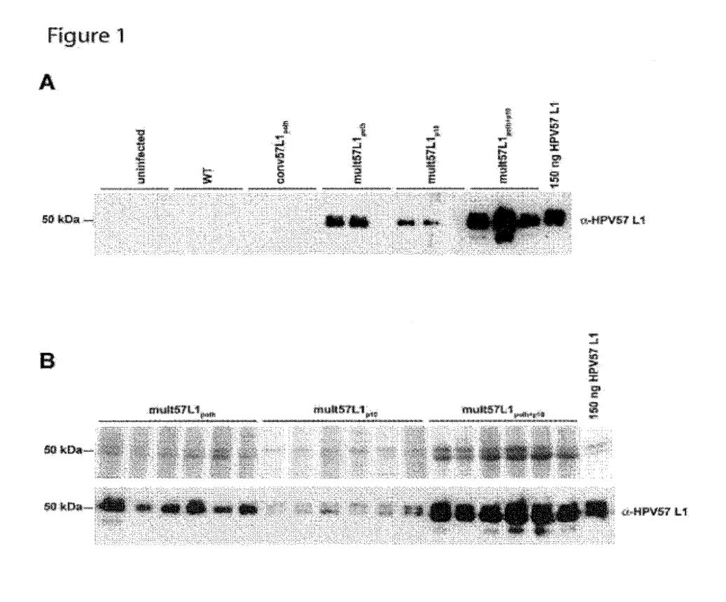

FIG. 1 Expression of HPV 57 L1 using the MultiBac expression system substantially increases VLP yield with high reproducibility. (A) Purified VLPs from infected insect cells. TN High Five cells were infected with a conventionally generated baculovirus transgenic for HPV 57 L1 (conv57L1ₚₒₗₕ), or with MultiBac-based virus carrying HPV 57 L1 in the polh-controlled cassette (mult57L1ₚₒₗₕ), in the p10-controlled cassette (mult57L1ₚ₁₀) or in both cassettes simultaneously (mult57L1ₚₒₗₕ₊ₚ₁₀). Three days post-infection cells were lysed and capsids were purified by a CsCl density gradient centrifugation. Equal volumes of the three gradient peak fractions per infection were loaded on an SDS-PAGE gel for immunoblotting using an L1-specific MAb. As controls, mock capsid purifications were carded out after WT-AcMNPV infection and after no infection and fractions corresponding to the peak after infection with mult57L1ₚₒₗₕ₊ₚ₁₀ were loaded. (B) To demonstrate reproducibility, six independent virus stocks were generated for each of the viruses mult57L1ₚₒₗₕ, mult57L1ₚ₁₀, and mult57L1ₚₒₗₕ₊ₚ₁₀. Independent TN High Five cell infections followed by capsid purification were carried out. Three peak fractions per CsCl gradient were pooled and loaded on SDS-PAGE gels, which were either Coomassie-stained and used for densitometric quantification (top) or immunoblotted using an L1-specific MAb (bottom).

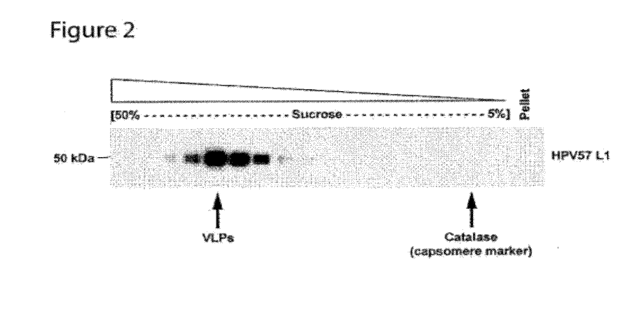

FIG. 2 HPV 57 L1 produced with the MultiBac expression system assembles into properly folded VLPs. Particles produced after infection with Multibac-based AcNPV recombinant for HPV 57 L1 in both expression cassettes were analysed by centrifugation through a linear sucrose gradient. The gradient was fractionated and samples of each fraction were immunoblotted probing with L1-specific MAb. As calibration markers HPV 16 VLPs and catalase, a marker for capsomeres, were used.

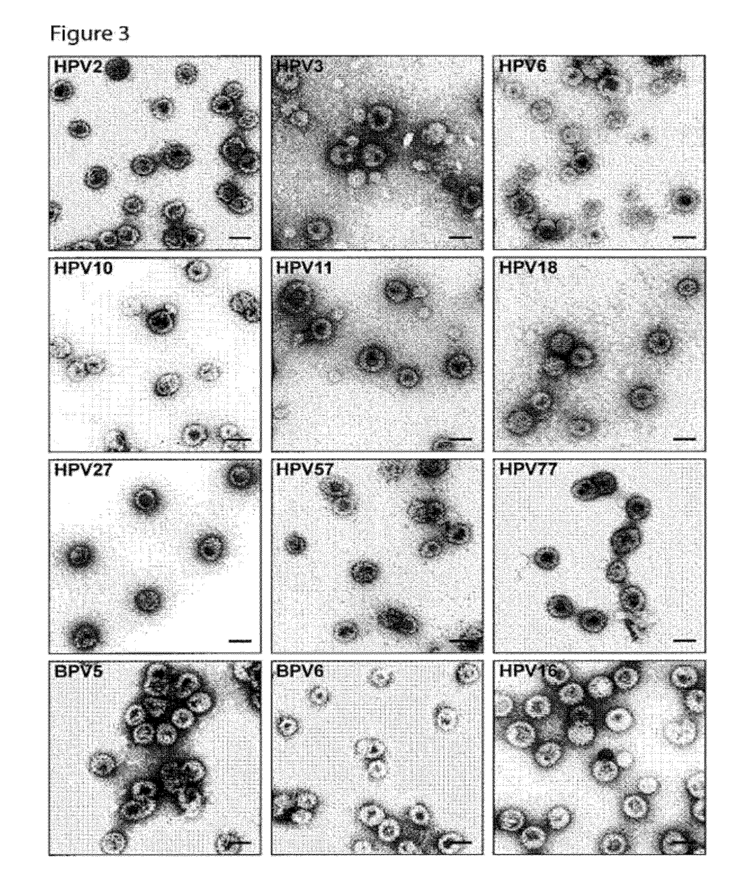

FIG. 3 MultiBac expression system permits VLP production of various PV types. VLPs from HPV 2, HPV 3, HPV 6, HPV 10, HPV 11, HPV 18, HPV 27, HPV 57, HPV 77, BPV 5, and BPV 6 were produced upon infection with MultiBac-based AcNPV recombinant for the respective L1 gene in both multiple cloning sites. VLPs were purified by CsCl gradient centrifugation and heparin affinity chromatography. Samples were analysed by electron microscopy. As control, conventionally generated baculovirus inducing HPV 16 L1 expression was used for a parallel infection followed by the same purification protocol. Bars indicate 50 nm in all panels.

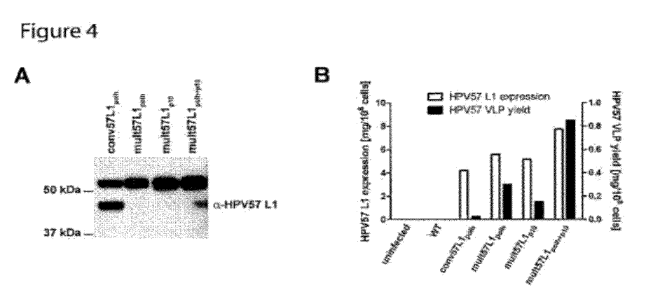

FIG. 4 A slight increase in HPV 57 L1 expression levels strongly enhances VLP yield. (A) TN High Five cells were infected with a conventionally generated baculovirus transgenic for HPV 57 L1 (conv57L1ₚₒₗₕ), or with MultiBac-based virus carrying HPV 57 L1 in the polh-controlled cassette alone (mult57L1ₚₒₗₕ), in the p10-controlled cassette alone (mult57L1₁₀) or in both cassettes simultaneously (mult57L1ₚₒₗₕ₊ₚ₁₀). Three days post-infection L1 expression was determined in cell lysates by western blotting. (B) Quantification of HPV 57 L1 expressed three days post-infection and of HPV 57 VLPs after subsequent capsid purification. Protein amounts were quantified by densitometric means after SDS-PAGE analysis.

### EXAMPLES

### Example 1

### Cells and Viruses

Spodopteria frugiperda 9 (SD; Invitrogen) cells were grown in suspension at 27° C. and maintained on Grace's insect medium (Gibco) supplemented with 10% foetal bovine serum (FCS; Sigma) and Pluronic F-68 (Sigma). Trichoplusia ni (TN) High Five cells (Invitrogen) were cultivated in Ex-Cel™ 405 serum-free medium (SAFC Biosciences) at 27° C. The WT AcMNPV was obtained from BD Biosciences, the recombinant viruses were produced as described below.

### Example 2

### Generation of Baculovirus Recombinants

All point mutations in the L1 genes were introduced using the QuikChange® Multi Site Directed Mutagenesis Kit (Stratagene). The generation of the chimeric HPV 16 L1E7₁₋₆₀ construct has been described previously (Muller et al., 1997). Full-length or mutated L1 genes were cloned into the transfer plasmids pVL1392 (Invitrogen) or pFBDM (Berger, I., Fitzgerald, D. J., and Richmond, T. J. (2004). Baculovirus expression system for heterologous multiprotein complexes. Nat Biotechnol 22(12), 1583-7) by PCR amplification with primers introducing restriction sites. All constructs were confirmed by DNA sequencing.

Recombinant AcNPVs referred to here as produced with a conventional system were generated as described in (Muller, M., Gissmann, L., Cristiano, R. J., Sun, X. Y., Frazer, I. H., Jenson, A. B., Alonso, A., Zentgraf, H., and Zhou, J. (1995). Papillomavirus capsid binding and uptake by cells from different tissues and species. J Virol 69(2), 948-54). Briefly, 2 μg of the respective transfer plasmid and 0.2 μg of linearized DiamondBac™ baculovirus DNA (Sigma) were cotranstected by calcium phosphate precipitation into 5×10⁶ Sf9 cells.

To generate the MultiBac recombinant AcNPVs, the strategy explicitly outlined previously (Fitzgerald, D. J., Berger, P., Schaffitzel, C., Yamada, K., Richmond, T. J., and Berger, I. (2006). Protein complex expression by using multigene baculoviral vectors. Nat Methods 3(12), 1021-32) was applied. Briefly, 10 ng of recombinant plasmid were transformed into DH10MultiBac cells. Positive clones, as identified by blue/white selection, were amplified and MultiBac bacmid DNA was isolated. One microgram of bacmid DNA was transfected into 5×10⁶ Sf9 cells by calcium phosphate precipitation.

All recombinant Ac viruses were amplified at least three times before their employment for a productive infection of TN High Five cells. The titer of all AcNPVs was determined by a plaque assay as described previously (Matsuura, Y., Possee, R. D., Overton, H. A., and Bishop, D. H. (1987). Baculovirus expression vectors: the requirements for high level expression of proteins, including glycoproteins. J Gen Virol 68 (Pt 5), 1233-50).

### Example 3

### Virus-Like Particle Production and Purification

PV virus-like particles (VLPs) were produced as described in (Muller, M., Zhou, J., Reed, T. D., Rittmuller, C., Burger, A., Gabelsberger, J., Braspenning, J., and Gissmann, L. (1997). Chimeric papillomavirus-like particles. Virology 234(1), 93-11). Briefly, 2×10⁸ TN High Five cells were infected with WT or recombinant baculovirus at an MOI of 2 unless indicated otherwise. Three days post-infection, cells were harvested and lysed by sonication. Subsequently, the lysate was cleared by centrifugation, layered onto a two-step gradient with 14 ml of 40% sucrose on top of 8 ml of a 57.5% CsCl solution, and centrifuged for 3 hours at 96,500×g at 10° C. in a SW32 rotor (Beckman). The interphase was collected and transferred into a Quick-seal tube (Beckman). A CsCl gradient was produced by a 16 hour-centrifugation at 184,000×g at 20° C. in a Sorval TFT 65.13 rotor and fractionated into 1 ml specimen. Purity and L1 content of the collected fractions were assessed by SDS-PAGE and Coomassie-staining.

The peak fractions were pooled, dialyzed against 50 mM Hepes (pH 7.4, 0.3 M NaCl), and cleared from residual debris by centrifugation at 20,000×g for 10 min at 4° C. The samples were further purified by affinity chromatography using 1 ml HiTrap™ Heparin HP columns (GE Healthcare). Elution of VLPs was carried out with 50 mM Hepes (pH 7.4) containing 1 M NaCl. The eluates were analysed by SDS-PAGE and Coomassie-staining and western-blot analysis. The capsid quality was verified by electron microscopy.

### Example 4

### Detection and Quantification of L1 Proteins

PV L1 proteins were analysed by SDS-PAGE and stained with colloidal Coomassie dye (GelCode Blue stain reagent, Pierce) or immunoblotted and probed with the anti-L1 monoclonal antibody (MAb) MD2H11. L1 protein concentrations were determined using image densitometry software ImageJ and bovine serum albumin or HPV 57 L1 as standards for the Coomassie-stained SDS-PAGE gels or the immunoblots respectively.

### Example 5

### Sedimentation Analysis

Samples were loaded onto a linear gradient of 5%-50% sucrose in 50 mM Hepes (pH 7.4) containing 0.5M NaCl and centrifuged at 222,000×g for 3 h at 4° C. using a SW41 Ti rotor. Fractions (600 μl) were collected from the bottom of the gradient and analysed by SDS-PAGE and immunoblotting.

### Example 6

### Electron Microscopy

VLPs (100 ng) were applied onto carbon coated grids and stained with 2% uranyl acetate. Grids were analysed using a transmission electron microscope CM200 FEG (FEI) operating at 200 kV. Pictures were taken at a 27,000 fold magnification using a 2 k×2 k CCD camera.

1-14. (canceled)

15. A method for manufacturing papillomavirus like particles (PV-VLPs), comprising the steps of:
(a) culturing a host cell lacking protease activity and comprising an expression vector, wherein the expression vector comprises at least one polynucleotide encoding a PV L1 polypeptide, and
(b) obtaining VLPs from the host cell.

16. The method of claim 15, wherein the PV is selected from the group consisting of human papillomavirus (HPV)-2, HPV-3, HPV-10, HPV-27, HPV-57, HPV-77, bovine papillomavirus (BPV)-5, and BPV-6.

17. The method of claim 15, wherein the protease is a member of the cathepsin family of proteases.

18. The method of claim 15, wherein the protease is a v-cath protein.

19. The method of claim 15, wherein the expression vector is a MultiBac vector.

20. A host cell lacking protease activity and comprising an expression vector, wherein the expression vector comprises a polynucleotide encoding at least one PV L1 polypeptide.

21. The host cell of claim 20, wherein the host cell is an insect cell.

22. The host cell of claim 21, wherein the host cell is a lepidopteran cell.

23. The host cell of claim 20, wherein the host cell is selected from the group consisting of Sf9, Sf21, Express SF+, and BTITn-5B1-4 (“TN High Five”).

24. A method for the manufacture of a pharmaceutical composition for the treatment or prevention of PV-related disease comprising the steps of the method of claim 15, and the further step of formulating the VLPs as a pharmaceutical composition.

25. The method of claim 24, wherein the PV is selected from the group consisting of human papillomavirus (HPV)-2, HPV-3, HPV-10, HPV-27, HPV-57, HPV-77, bovine papillomavirus (BPV)-5, and BPV-6.

26. An expression vector comprising at least one polynucleotide encoding a PV L1 polypeptide and lacking a functional gene for a v-cath protease.

27. The expression vector of claim 26, wherein the PV L1 polypeptide is selected from the group consisting of L1 polypeptides comprised in human papillomavirus (HPV)-2, HPV-3, HPV-10, HPV-27, HPV-57, HPV-77, bovine papillomavirus (BPV)-5, and BPV-6.

28. The expression vector of claim 26, wherein the expression vector is a MultiBac vector.


SEQUENCE LISTING (40 sequences)

SEQ ID NO: 1 - protein, 323 aa, Autographa californica nucleopolyhedrovirus [sequence omitted]

SEQ ID NO: 2 - protein, 323 aa, Epiphyas postvittana nucleopolyhedrovirus
MSKFLLYWFVYGVVCSAAYDILKAPNYFEEFVRQYNKQYDSEYEKLRRYKIFQHNLNDIITKNRNDTAVYKINKFSDLSKDETIAKYTGLSLPLHTQNFCEVVVLDRPPGKGPLEFDWRRFNKITSVKNQGMCGACWAFATLASLESQFAIAHDRLINLSEQQMIDCDSVDVGCEGGLLHTAFEAIISMGGVQIENDYPYESSNNYCRMDPTKFVVGVKQCNRYITIYEEKLKDVLRLAGPIPVAIDASDILNYEQGIIKYCANNGLNHAVLLVGYGVENNVPYWILKNSWGTDWGEQGFFKIQQNVNACGIKNELASTAEIN

SEQ ID NO: 3 - protein, 325 aa, Clanis bilineata nucleopolyhedrosis virus
MKTFLLFFAIITSSVCGYDLLKAPDYFESFVANYNKMYNDTQEKAYRYKIFKHNLEEINIKNQVEDHAVFSINKFSDMSKSEIISKYTGLSLPSLMQENFCRAIILDGPPNKAPINFDWRQYNAVTPVRVQGNCGSCWAFSTLAGIESQYSIKYNKQISLSVQQLVDCDTSNMGCAGGLLHTALEQIINAGGGVLQEEDYPYKGVDKQCNLPHNNFAVQVLGCYRYIVMNEEKLKDVLRAVGPIPVAIDAASIVDYSRGIIRTCTYYGLNHAVLLVGYGVQDGVPYWTLKNTWGDDWGEHGYFRVRQNVNSCGIINDLASTAVIK

SEQ ID NO: 4 - protein, 5 aa, Artificial
  synthetic
RGFFP

SEQ ID NO: 5 - protein, 4 aa, Artificial
  synthetic
AAPM

SEQ ID NO: 6 - DNA, 7904 bp, Human papillomavirus type 16
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 7 - DNA, 7746 bp, Human papillomavirus type 5
aacggtaagttgcaatttccttgtaccaggtgcggtattgggatttcacaattataatggttgttgccaactaccataggcatattcaagtttttgcctgtatcgttttcgtatcctgtaataatatccaatatatgtatacataaataaatatatatatatataagtgtctaagattgggttcttctgtaatcaggcaatggctgagggagccgaacaccaacagaaactgacagaaaaagataaggcagaattacctttaagtattagagacttagctgaagccttaggcatccctgtgattgattgtttaataccttgcaatttctgtggcaactttctaaattatttggaagcttgtgaattcgactacaaaaggcttagtctaatttggaaagattattgtgtgtttgcgtgctgtcgcgtatgctgtggcgccactgcaacttatgaatttaaccaattttatgagcagacagtgttaggaagagatattgaattagcttcaggactttcaatatttgatattgatatcaggtgtcaaacttgcttagcatttcttgacattatagaaaagttagattgctgtggcagaggccttccctttcataaggtgaggaacgcctggaagggaatctgtaggcagtgtaagcatttttatcatgattggtaaagaggtcaccgtgcaagatattattctggagctcagtgaggtgcagcccgaagtgctaccagttgacctgttttgtgaagaggaattaccaaacgagcaggaaacggaggaggagcctgacaacgaaaggatctcttacaaagttatagctccgtgcggttgcaggaactgtgaggtcaagcttcgcatttttgtccacgccacagaatttggtattagagctttccaacagctactgaccggagatctgcagctcctgtgccctgactgtcgcggaaactgcaaacatgacggatcctaattctaaaggtagtacatctaaagaagggtttggtgattggtgtttattggaagctgactgtagtgatgtagaaaatgatttgggacaattatttgagagagatacagactctgatatatcggatttgttagatgatactgaactggagcagggcaattccctggaactatttcatcaacaggagtgtgagcagagcgaggagcaattgcaaaaactaaaacgaaagtatcttagtccaaaagctgtcgcacagcttagtccgcgacttgagtcaatttcattgtcaccccagcagaagtctaagcgaaggctctttgcagagcaggacagcggactcgagctgactttaaacaatgaagctgaagatgttactcctgaggtggaggtaccggctattgactctcggccggatgacgagggaggttcaggggacgtagatatacattacactgcattgttgcgttctagcaacaaaaaagctacattaatggctaagtttaaagagtcgtttggagtaggttttaatgaattgacacggcaattcaaaagccacaaaacctgctgtaaggactgggttgtctctgtatatgcagtgcatgatgatctatttgaaagctcaaagcagctattgcaacagcattgtgactatatctgggtccgtgggataggtgcaatgtcattatacctattgtgttttaaggcgggaaaaaatcgcgggacagttcataagttaattacctcaatgttaaatgtgcatgaacagcaaatattgtctgagccgccaaaattgagaaatacagccgctgcattgttctggtataagggttgtatgggatcgggggcgtttagccatggaccatatcctgattggattgcccaacaaactatattaggtcacaaaagtgctgaggcaagtacttttgatttttcagcaatggtccaatgggcatttcataatcacttattagacgaagcagatatagcataccagtatgcaaggcttgctcccgaagacgcgaatgcagtagcttggcttgcacataacaaccaggccaaatttgtgagagaatgtgcatatatggtacgattttataagaagggacaaatgagagacatgagtatatctgaatggatatacactaaaatcaatgaagtagaaggggaagggcactggtcagatatagtaaagtttattagataccaaaatataaactttattgtattcctaactgcattaaaagaattcctacactcagtgccaaaaaaaaattgcattttaatttatggtcctccaaattctggaaagtcatcatttgcaatgtcattaataagagtgttgaagggtagagtgttgtcatttgtaaattctaaaagtcagttttggctgcaacccctttcagagtgcaagatagctctattggatgatgtaacagacccttgttggatatacatggatacatatttaagaaatggcttggatggacattatgtttcattagattgtaaatatagagccccaacgcaaatgaaatttcccccattattattaacatctaacattaatgtgcatggggaaactaattatagatatttacacactacaataaaaggatttgaatttccaaatccttttcctatgaaagcagataatacacctcagttcgaactaactgaccaaagctggaaatctttttttacaaggctttggacacaattagacctgagtgatcaagaagaggagggcgaggatggagaatctcagcgagcgtttcaatgctctgcaagatcagctaatgaacatttatgaagctgcagaacaaacattgcaggcacaaattaaacattggcaaaccttacgaaaagaacctgtattactctactatgctagggagaaaggtgttacaaggcttggatatcaacctgtgcctgtaaaggcagtatcagaaacaaaggctaaagaagccatagcaatggtgctgcagcttgagtcactacagacatctgattttgctcatgagccatggactctagttgataccagcatagaaacatttagaagcgctccagaaggtcacttcaaaaaaggccccctccctgtagaagttatttatgacaatgatccagataatgccaatttgtatacaatgtggacctatgtgtattatatggatgcggatgataagtggcataaggcaagaagtggggtgaatcacattggcatttattatttacaaggaacttttaaaaactattatgtactgtttgctgacgatgcgaaaagatatggtacaactggagaatgggaagtaaaagttaataaggaaactgtgtttgctcctgtcaccagctccacgcctccagggtcgccaggaggacaagcagacacaaacaccacccccgcgacccccaccacctccacaaccgccgttgactccacgtccagacagctcaccacatcaaaacagccacaacaaaccgaaaccagaggaagaaggtacggacggaggccctccagcaagtcaaggagatcgcaaacgcagcaaaggcgatcaaggtcccgacaccggtcccggtctcggtcccggtcgcggtccaagtcccaaacccacaccactcggtccaccaccaggtcccggtccacgtcgctcaccaagactcgggcccttacaagcagatcgcgatccagaggaaggtccccaaccacctgcagaaggggaggtggaaggtcacccaggcggcgatcaaggtcaccctccacctcctcctcctgcaccacacaacggtcacagcgggcacgagccgaaagttcaacaaccagaggggcccgagggtcgagagggtcacgaggagggagccgtggggggagagggcggcgacgaggaaggtcatcctcctcctcctcccccgcccacaaacggtcacgaggggggtctgctaagctccgtggcgtctctcctggtgaagtgggagggtcacttcgatcagttagttcaaagcatacaggacgacttggaagattactggaagaagctcgcgaccccccagtaatcattgtcaaaggggcggctaacacactgaaaaatgtccgcaacagagctaaaattaaatacatgggactgtttaggtcatttagtactacctggtcatgggtggcaggagatggcactgagcgtctaggcaggcccagaatgctcattagcttttcttcctatactcaaaggagagattttgatgaagcggtgcgataccccaaaggagttgataaggcctatggcaacctggacagtctttaacatttactaatgctgcttttgctactaacatactaacataccctagcattttatatttttttttacattttgtatttgctatggcgcgtgcaaaaacggtcaagcgagactctgtaactcatatttaccaaacctgcaaacaggcaggcacttgcccccctgatgttattaataaagtggaacaaacaacagttgctgacaatattttaaaatatggcagtgctggtgtattttttggtggccttggtattagtacaggccgaggaactgggggtgctacagggtacgtgccacttggggaaggtcctggtgtccgtgtcggaggaacccccacggttgtaaggccttccttggttcctgaaacaatcgggcccgttgatattttgcccattgatacagttaaccccgtggaacctacagcatcatccgtggtccctctaactgagtccacaggcgctgatttacttccaggtgaagtagaaacaattgctgaaatccatcctgtacctgaggggccatcagtggatacccctgtagttaccactagcacaggttccagtgctgttttagaggttgccccagagcctattcctccaacacgggtcagggtttcacgcacacagtatcacaatccatcttttcaaataataactgagtctactccagcacaaggggaatcgtctcttgcagatcacgttttggtgacatcgggttctggggggcaacgaatagggggtgatataactgacataattgagttagaggaaattcctagtaggtatacatttgaaattgaagaaccaactcctccacgccgcagcagtactccattgccacgcaatcaatctgtaggccgtaggaggggtttctctttgactaatagacgtttagtacagcaggtacaagtggacaatccattgtttctaactcaaccatctaagttagttcgttttgcatttgataatcctgtttttgaggaagaagtgactaatatatttgaaaatgatctggatgtctttgaagaacctccagacagagattttcttgatgttagggaattgggacgtccacaatattctacaacaccagcgggatatgttagagtaagcaggttggggactcgagccactattcgcactcgctctggtgcacagatagggtcgcaagtccatttttacagagatcttagctctattaatactgaagatcctattgaattacaattattaggccaacattcaggtgatgctactatagtccacggacctgttgaaagcacatttatagatatggatatttctgaaaatccattatctgaaagcattgaagcatattcacatgatttattattagatgaaacggtggaagatttcagtgggtctcagctggttataggtaatcgaaggagcacaaactcttacactgttcctaggtttgaaactacaagaaatggttcatactatacacaagacacaaagggatattatgttgcatatccagagtcacgtaataatgcagaaatcatttatcctacacctgatattcctgtagtcattatacaccctcatgacagtacaggggacttttatttacatcccagtcttcacaggcgcaaacgtaaaagaaaatatttgtgatttgcattcgagatggcagtgtggcactcggctaatggtaaagtatatcttccaccatcgacaccggtggccagagtccaaagcaccgatgaatacattcaaagaacaaatatctactatcatgcatttagtgacagattgttaactgtaggtcatccttatttcaatgtatacaatattaatggtgataagcttgaggttcctaaggtttcaggaaatcaacacagagtatttcgcctaaaattaccagatcctaacagatttgcattacctgatatgtctgtttacaaccctgacaaagaacgtttggtttgggcctgtagaggcttagaaataggtaggggccagccattaggtgtacggagtactggtcacccttatttcaataaagtaaaagatacagaaaacagtaatgcatacataacattttctaaagatgacagacaggatacatcttttgatcctaaacagatccaaatgtttattgtaggatgcacaccttgcataggagagcattgggataaagctgttccatgtgcagaaaatgatcagcaaactggcctttgtcctcctattgaactaaaaaacacatatatacaagatggtgatatggcagacataggttttgggaacatgaattttaaggcacttcaagatagtagatcagatgtcagtttagacatcgtcaatgaaacttgcaagtatccagattttttaaagatgcaaaacgatatttatggcgatgcgtgctttttttatgctcgtagggagcaatgttatgccagacacttttttgttagagggggaaaaactggtgatgacattccacgtgcacaaattgacaatggtacatacaaaaatcagttttacattccaggggctgatggccaagctcaaaagactataggaaattccatgtatttcccaactgttagtggctcattagtatccagtgatgctcaattgtttaacaggcccttctggctccaaagagcccaaggtcataataatggcatcctgtgggctaatcaaatgtttatcacagtggttgacaacacaagaaatactaatttcagtatttctgtatataatcaggctggagcactaaaagatgttgcagactataatgcagatcaatttagagaatatcaaagacatgtagaagaatatgaaatatctttaattctacaactctgtaaggttcctttaaaggcacaggtattggcacagatcaatgcaatgaactcttcgttattggaggattggcagttaggatttgttcccactcctgataatccaattcaggacacctacagatatattgactctttggctacacggtgtccagataagaatcctccgaaagaaaaggaagacccttataagggcttacatttttgggatgtagatttaactgaaagattgtcattagatttagatcaatattccttaggcagaaaatttttattccaagctgggttacaacaaacgaccgttaacggtacaaaagcagtgtcttataaagggtctaatagaggaacaaaacgcaaacgtaaaaattgaggtctgaccgaaagtggtacatttttataaacttttacacagtattcaaggaatgtttgtttactctgactaagtataagtcttccaaggataccgaccgcacccggtacactcagtcaagttgttgccaatatagaatcagatcagtgccaaacacaccgtcttggactcagaacagaccgtgttcgttataacatgctcggattagggacctccccaaagaagatttaatctacaatcgcttttggcaatcgcatttggcactgctaaaagaccgtt

SEQ ID NO: 8 - DNA, 8374 bp, Deer papillomavirus
gttaacaataaccagaccttagccgtttttgggtgagcgggaaagatgggttacaggttctataaaagcaccacaccgcacaaggttgctatcacttgtcactctgctcagacccttccttctgcatgtctgctgattactatgaacatctatactgtgtattttgctactgtgttcttggaaaggtggaagctcgccgatgctatgacaaaaaaattagaacagtggtaagaggagggctcagatgtgcagtttgcactgcatgcttggaaaaagggctctatctggaaagagtgctgaatgcgcctcaacctgtatatcaggggtccattgaagagcctgatcctttcattcaaaaagcctgcataagatgcatgtactgtgggggaatactgacccgtgacgaaaaggacaggcacagatattttgaagagctttacgtgatattcaggaatcaggttcttggcagatgctacacctgtactaggcatggcatgtgctcggccccttaccgggcgaacgctaccggctgatgaatcaccttgcttaacattgattttggagccagtctcgggagaagcagccaagaacagtacaccagtcgttgtggataagcctggaaaaccgccccctaaacgccaccgaagacagtataatgtgactgtttcctgcaacgactgtgacaagcgtctgaacttctctgtcaaaactacctgcagcacaatactcaccctgcagcaactcctgacagaggacctggatttcctgtgttctttctgtgaggccaagaatggataaagaaaatgcaggtagctctggggttgggggggattcttttatcctctttgaggcagaatgctcagatacagattctgaatcacctgcccaaggtgaatctactgatgaggatttactagataatgccactgccgttccgggaaaccacctggagctcttccaaactcaggaaaaagaggcgggagaaagacagatttcaattttgaaaagaaaactgtgtttaagcccttgctctgctgactctgaggtggagcagctcaagtcctgggcttgctgtcataagtatcacacctcggaagcgaatcccgttgttagacgcaggcttttcgaaagaggtgatccaggcggtgctaacacacctgtgaaccatgaagctgacaatttttctccgtcaggactgcaggtacagtctggggaaaataggtggagccaggaaaagggaaaagggggagtttcgcccgtgcctagctcagctgagccaaatatggccgcctgcatacagaaattgttcaagactctctacatcgcctcccatggggagatcactcgtgtattccaaagtaataagactgttaaccatcagtgggtgattctggcatatggagttagtgaggtgttgtattctgctagctttgatctctttggtaaacagtgtaactgcctgcaaacgtccagaaaggttcatgaaaaagggagtatttctgtttaccgttgtatgttcaatgttgccaaaagtagagatacagtgcagaaattaatgaccacaattctgaatgttaccgcgggcaacctcctcctacagccccctaaaatcagaggtctcgggcctgctctattctggtttaagctcacactgtcacctgctaccttaacccatggtaccacaccggaatggatacagcaggcaactaatgttgccagcaatactggagaggcggctaaatttgatttaggaactatggtgcagtgggcttatgaccatggtttcacggaggagtcgaaaattgcttatgaatatgctctgtgtgctgggagcgactgcaatgccaaagcatttttggcaagcactagccaggcccgtttggtcaaagactgctgcaccatggtgagacattacctgcgtgctgaggtacaggccctgacaatgtcaggttatataaaaaggcggtgtgatcaaactgcaggaagtggcagctggctctctatcatgaatttgcttaaatatcatgggatagaacatatacagtttgtgaatgcattaaagccttggttaaaaggcattcccaaatataactgcattacaattgttggcccgcctaacagtgggaagtcactcctttgcaactctttgatagcatttcttggcggcaaggtgcttacatttgcaaaccaccacagccacttttggctcgcacccttagcggactgccgagttgctttaattgatgatgctaccacagcttgctggaggtactttgacacacacctcagaaatgtgttggatggctacccattcggtattgatagaaaacacaataccgctgttcaaatgaaagcccctcccctcttagtaaccagtaatattgatgtgcatgcagaggaaaagtatttctattcgcacagcagagttaagccgttttacttcaaggagccgtgccctgcttcagacaatggtgagcctatgttttctataactgatgctgattggaaacatttctttgaaaggctatggggacgtttagacctgagcgatcaagaggacgaggttgacgacgatgagtgcagccaaagaacagttacttgcagcgcaagaaacgcaaatgacattaattgaaaaagatagcacagatttgaaagatcacatagactcttggggtccggtcaggagagagcatggtttgctttatgctgccagacacaaaggcttgatttggctcggtttgaaccctgtgccaccatgctctgtgaagtgcttagaagctcggcaagcaattgagatgcagcttctggggaacagcttaaaggagagcccatggtgcaatgagccatggtcactgtgtgacttaagctggggacgctatcaagcgcctccagcagaaactttgaaaaaaggcgccagactggttgaagtggagtatgatgggagctccactaataaaacttggtataccgcttggaattcattgtacttgcgcaaaccggatgaggagggctgggagacggcgactggtggtgcagacgcagacggtctcttctatactaccatgtccggtacacgggtttattatgagctctttgaaagagatgcagccagatacagcactacagggacttggactgtgagggataacgatcgtacttatcactcacattctgcgccctcccactctagagagaccatcgaaggactgtggaactccgggggccgtgaaagaggcagacccaccaactcgcccgaccgcgccgtgcttcacactcctcctggaggcaacaccgttcacggtcccgtcagagcttgcgaaaaccggggtcggtccattaaccgcccgactccctacagcacaccacagtccccgaggagtggcgtgggccccgataccacctccccgctgccgagcccggtaccgcagaacccccggtgcgtatctctacccgacggttttggacgaggggaggaggataacccgccgtcgccagatcaacacgacgtaatccccaacccccagccgaaagaaccgcggtttagcctatttggctcttcaggtgggctgccctgtcttctaattagtggaactgggaaccaagtcaagtgctattccttccgcgtgaaaagatggcatagggacaagtatcaccactgcacgaccacctggtgggcagtcggggagcagggatctgaaagaccaggcgatgccacagtgatcgtcaccttcaaagaccaaagtcagagatcaatgtttctgcagcaggtgcccttaccgcctggtatgtcagcacatggagtgactatgactgttgacttttgagtctgttgtgctactggctgctatctgctgcagcatcgcgagtctgatactttacctgtggaaaatggaaacgctgcatgctgtaaagcatgaaccatcctgggctgtttctgtttctgggactggtctttggagtgcagttgctactgttagtatttattttgtttttcttctttgtatggtgggatcagtttgggtgtaagtgtgaaaacttccacatgtaaatccctttcctgcatggagggatcaggtatggtgtctctgtgaacacttttagatgtaaataatcatttgatccaaagctttgtaaatagcaataggtgcaatttaaggagcattatttactggcgcatcgggcagatttctgtgtcatcccaaggagccttctctctctctctagttctctcacgtggtacagcataatcctccttcctttatttatttttgatattattcatattattcatatatagagctattattatccaaggtcaggtactgttatccaaggtcagcaagatcagttcccaagagttttcttctttcgaccctcttcgagttggactgatacggactatggactatggactaaaacttaagtgcgtgtgcgacgatcaccagggggccattcttgttttgttttttttcttttgttgttttgttctcgagtaacctcaagcctttgtcagacagaaccattagtacagcggtgccatatctttgaactgccaaaaaaaagacacataagcctgcatgaaactttggttgttattgttgctgctattattattaggtcattggacaccagtttgggttatattttgtttgctgtggtcgagcttcctatttttgttacattctgttttcataactttttgacagggctgtttcagcttcccaatcaaagtttaagtgtctgtgtttacaataaatgcaccatgccaccattaaagcgtgtgaagcgtgcaaatccatatgatctctacaggacgtgcaagaggtggaagtgcccactgatgtcattcctgaaggtggagggaaaaactgttgctgataaatactgcagtatgggcagtatgggtgtatactggcggctggcattgcatgggagtggccgcccaacccaaggtgggtatgttcctttgaggggaggtgggtcctctacatctctttctagcaggggaagtgggtcctctacatctatttctaggccctttgctggagggattcctttggaaacgctggaaacagtgggtgcttttcgccctggcatcatagaggaggttgctccgacactggaaggtgtcctacctgatgctcctgcagtggtgactccagaggctgtacctgtggatcagggcctgagtggcttggatgtggcaagggaagtcacacaagaaagcctcatcacttttttgcaaccagaagggccagatgatatagcagtgctggaacttaggcccacagagcatgatcaaacacacctgatttccacctcaacgcacccaaaccctttgtttcatgcacctattcagcagagcagcattatagcagaaacctcagggtctgagaacatatttgtgggtggtgggggagtgggtagcaccactggggaggagattgaactcacactgtttggtcagccaaagactagcactcctgagggccctattaaccggggtcggggcattttcaactggttcaacagaacatactacacacaggtacctgtggaagacccagacgagattgctgctgcaggctcgtatgtctttgagaatgcattatacgattcaaaggcctacaaacatgagcagcagccgtggttatcgcgaccacaggatgcacctgagtttgacttccaagatgcagtgaggctacttcaaggaccctcgggtcgtgtggggtggagcagaattattaggcctacctcaataggcacacgttcaggggtacgggtggggcctttgtatcacctgcgtcagtcgttcagcactattgatgagcctgaaacaatagaattgattcctagcactgttgatgaagaggaggttttgactggagtacctgagtcggctgaaggacctgatgcagaatattcagatattgatcttcaaagtataggaagcgatgagccccttttagggacaggcatcatctatcctttggtgggcggaggacaaatattcctgtgcatgcacagggccccagtgggttggtcctcagggacatacattaatcatgaaggacaaagtagggatgatggtgagtacgtgatagacaatggaggacaatcgaacatcacgcctactgtagttattgatggatcaattgctttgtctttggaatattttaggcattactacctgcacccaagtctgctaagacgcaagcgcaaacgcaacccaatatttatctgatgttttgcagatggcgttctggcagcctggtcaagcgctatacctgcctccaacacctgtgacaaaggttctttgctcagagcagtacattaacgtacgggatatattttatcatggggaaacagagcgcatgctcaccagtgggtccatcctttctcttgaggtgacacagaagcacacgactgtccctaaagtgtctccaaatcaatacagggtgtttagagtcgcattacctgatcctaatcagtttgctttacctgataaggcccttcataaccctagtaaagaaaggttagtgtgggcagttgtgggtgtccaggtgtcaagagggcagcccctgggtggggaggtaagagggcattcctacttcaatactttcttggatgcagagaatgtcagtaaaaaagtaactgctcagggcaccgatgaccgtaagcaagcaggtatggatacaaaacagcaacaggtgctgatgcttggatgcactcccgctataggtgagtactggacaaaggctcgtccatgtgtaacggacagaccagatgctgggtcatgtcctcctattgaattaaaattaagctttatagaggatggagatatgatggacataggatttggggctgcgaactttaaagagttaaatgccacaaagtcagatctacctctggacattgccaactccatctgtttgtatcctgattacttaaagatgactgaggaggcggcaggcaatagcatgtttttttttgccaggaaggaacaagtttatgtaagacacatttggaccccgtggggtactgacaaagaactcccacccgaggcctattatctgaagccaccgggggagatggaactcaaaatgccaagtgttttctttgcaagtccaagtgggagtttagtatctacagatggccagctattcaatcggccatattggatactgagagctcagggaatgaacaatggtgtatgctggaataataccctatttgtgacagtgggagacaacacaaggggcagcacactgaccatcacggtccctaacaatgatgagcctttgacggagtatgatactagtaaatttaatgtatatcaaagacatgtggaggaatttaagcttgcattcattctcgaactatgctcagtggagcttactcctgagactgtctctagtctccagggctcaatgccctcaatcctggaaaactgggaaattaacctgcaacctccaacatcctctgtgttagaggatatctatcgctttatagattcccctgcaacaaagtgtgcagataatgtatctcccagcaagcctgaggacccatactctgctcataagttttgggaggtaaacttaaaagagaaattatctttagatttggaccagtttcccttaggtcgcctcgtcctacagtttgactgtcgtctagacaggcttttacctcaaaaagaccacttcacgtaccctgaaaagcggtataaacggcacatgaggataacggggacggtgagaaaagtgcttctgtacatatgctttagtttaaattcctaataaacctgagtttcttgaatgtgtctagtcatgttccaattgagtgtgtcatgtcctgcttgtattcctccgaagtttgcaccacactcccggtgtcggaatgaagctgataatacccttgagacgcactgtcagcaacttttaattcacagcaggcctcttggctcggcacattgacgccgcagacagctgcatttgtctggattagggataccgctggcgttcaataaagcacttcgtctgcaactttcacggtaccggcgcgcacgtcgagtccgaaccgctgacggtggttatggtaagtaggcggtcaatagaggcggaactgaggcggaacttatagactataagccggacatgtccttgcaagttcaatgcctatagaaccgtcctcggtgctgtacaaggcgctaaaccgttcgcggtgtagtgtttaatgcatgccatcgcgagcggtctcagctgatcttcttaagttcccaggcgtcttgattggacggttccaaatgtactgaagtactttgcaggcatcgctctcggtacgtgagaatcttgatttttcagtgtgaatgattgtt

SEQ ID NO: 9 - DNA, 7841 bp, Bovine papillomavirus type 5
atgccatactggttgcccacatataacttcgaaggactgcaatgcttgcagtgcaagaaagctttagggtccctggatgctctaaaatgcaagaatcataaatataggagggtgcatagagggggaaagccttatggcatgtgtcaaatttgcttagaggctttgctgcaattagaaaggcaagaatttccttggacattgctactgccaaaggactttgttaaagttttggggagactgcctggagactattgtgtacgctgctattactgtggctgcgtgttgtcagacagtgaaaaagatcgccacgccttggaccacgaaggttacctgtacgtccgtggaagagccagaggccgctgctactcttgctctagtgatggtcgccggccctgcgtgttctaaatttctgccacaagaccctccaccaccctcagtgacactggttcttcatgatttgactcaagaagaggatgaacaggattttgtaacgctgcacgcacaatatagaccaacttttaaagataaaactcctagacgacctggctacaatcctcgtgctgctccctgccacattcagaggctctctgcgaggtgctcagttttttttgtgcaagtcggatgcccctgtggtcagcctttgaagattgctgtgcaaagtaccccagactgtatctctcagtttgaacaacttctgcgaggacctttagatcttctgtgtcctcactgcgcatcccgctgctatggcagataaatcaggtagattgctggggggctgctcttttgtattagatgagggctgactgtagtgatcttgaaatagatagtgatgatgagtctgataaggaaaatgtgccaaatggacaggatatgtgcaatagcttcgatgctgaatttatagacaatgcgcctttagcacagggaaataccctggcccttttccagagccaggtagcccaggcgggaaaacagaaagtaaactatctcaaaagaaaactgcacctcgagtcgagtgagctgggcacggttggtagagcagtgctgcagcctgtgaaccacagcaccccagcagctaaaaggcgcctttttgagtgctcaagtagtgaaaatgaagttagttatgctgcttcgcccgccgccgcaaacacacaggtatttagaaatcaaaatagtgggtctgtgggaggaagtagcgggtttgggtcacaggcttcggtaagtcagtctcaacaaaacagtaatttacatttgcagattttaaagtctaaaaatagtgctgcttgcaagcttgctgtatttaagtttgtgtatgctgcgagtttttgtgacttaactagaccttttaaaaatgataaaacaacaaactatcagtgggtggcggcggtctttggggtttcggaggagttgtttgaagctagtaagcagttgctaggtagaagctgcacatatttgcatgcgacctgcagagcccatgaaaagggctcagttgctttgcttttattatcctttcacgtggcaaaatctagagagacagtcacaaatctgttaaaaaatttgctcaatttaagagctgagcacatgatgctgcagcctccaaaacttagaggggtaacatctgcaatgttttggtataaaatgacattaagcccgaatacttatacatgggggcagttacctaggtggatagaacagcaaatattaattacagaaaatagttcagaagttttaaaatttgatttctctcacatggtccaatgggcccttgataatgagatgatggatgagtcctccatagcttttcattatgcgcagatggctgatcatgactctaatgccagagcatggctaggtttaagtaatcaagctaagatagttaaagatgtctgcactatggtacatcattatcagagagctataatgcgtagcatgacaatgtcagcatatgtgcacaaaatgtgtgaaagagtaaatgtgactgggtcttggttagtgatcatgcagtttttgaagtttcatggaattgagccaataagatttgtgaatgccttgcgcccatggcttcaaggagtgccaaaaaaaaactgtcttgcatttatagggccacctgatactggcaaatctttattcactaatagcctgatgagttttctaaaaggcaaagttttaaattttgcaaatagtgcaagtcacttttggctggcccccctgactgaagccaaggtagctttaatagatgatgccacgcatgcctgcttaaaatactgtgatacttaccttagaaatttttttgatggttattctgtgtgcattgataggaagcataaaaatgcagtccaaataaaagcacctccaatgcttttaactagcaatatagatatacaggcagaagaaaagtattcttacctcaaaagcagggtgacctgcttctattttaatgataaatgtcctctaaatgaagatggaaaaccactgttccaaataactgaccccgattggaaatctttttttgaaaggctttggcagcgtttagagctcagtgaccaggaggaggaggaggagggggacgaaaatggcagccgcggaacgtttatctgcagcacaagaaactcaaatgactttacttgagaagcctagctttgatttaaaagatcatatatcatattatggggctctgcgaacagaaaacactattttttatgcagctcgcaaaaaaggtctgacctcacttggacactgtccagttcctaccctggcaactgcagcagccaatgcaaaagcagcaattgaaatgcagctgctgctaaaagacttgttacgttcaccttttgccaaaaatgattggtcactcaacgatgttagccatgagcgctacaaggcccctcctagtgacactttgaaaagaaagcctagaattgtggaggttatatttgataaggatccacagaataaaacctggtacactctatgggatgaagtgtacgtatgcactgtagatgggtggactttaaccactagcggcgctgacgccacaggcatatttgtcaatatgcagggcagccggcagtattatgagcttttcggagaggacgctcagaggtttgggacctctggaacgtgggaggtcatagatcaaaaccaacggtttcattttccaccatcctctagagcggacaccacagacggtctgcctggactccaagaggaacctcgaggaggagacggacctacctgctccggacctgctccagccatccctgactctccttcacgttgcttatccagaggttttgcaggaagagatcctggctgtcaccgtaacagacaccgcgtacatccctacattttatcaggaggccaaaggatactggtcacttccagttcatcatccacggtgcaaggccctctttcgtcgggttcttcacaacactcgcaaagccgcggtcgcccgccatcaccggattcaacggaaacagaaagggctcggacaccagtaaactctgaccgccagcgtgcaggagaatttgacttgctgaaagggggctgcagaccctgctgcttaattgaaggtaatggcaacaaggtgaaatgcttgcgcttcaggcttaaaaaaagtcatcgatccaggtttctggacatcactactactttttgggctacaggggatgagggctcagacagacaaggcaatggaactatcctaatcactttcactgatactactcaaagggacttatttcttggaagtgtctctattcctggtgaactttcggttcgacgcattacaatctctacagactgaactttagccatgcaatcttgcaggacaatggtctgcagagacccttaatgttttgtctgtatgttttcttaatttttccacatttagaatgtactttatggtgctgtgacttgctgctgtgtatcattatattgtggcttttttatatgtgtttaaacaatttgtaacttttatactttttcagtaaagttttaaacatggctgtgcgtctgcgtagagtcaaaagggcaaatccttatgacctatacagaacctgtgctactggtgactgtccccaagatgttaaggacaggtttgagcataatacaatagctgacaagatacttaaatggggaagtgcaggagtgtttttagggggcttgggtataggcagtacacaagctaggccagggcttggaacctattccccacttgggcggggaggtgttactgggagaatccctgtcagggggccaggaagcaccagacccttaggaagaccatttagctcaggccccatagatacaataggagcaggggttcgtacatctgtagaaactagtgtgactgtccctgatgtagtggctgtcctgccagaatctccagcagtaattacaccagatagtatgcctgtagaccctggagtggggggtttggatattagtgcagaaattatagaggaaccatcattgacttttgtggagcctcatggaccagaagatgttgctgttttggatgtgaatcctgcagagcatgatcgcagtgtgtatttatcttctagcaccacacaccataacccctcttttcaaggtcaagtgacagtgtacactgatattggggaaacttctgagactgaaaatttactgattagtggcagcaacatagggtcgagcaggggagaagagattcaaatgcaattattttcagggcctaaaaccagcacacctgaaactgatgcagtgactaaggtaagaggccgagctaattggtttagtaaaaggtactatactcagaccagtgtgcgagaccctacctttattcaagagccacaaacatatttctatggttttgaaaatccagcttatgagccagatccatttgaggatagttttgacgtgcaattagccagtccatctgaacctgtacagcctgaacttagagatattacacatgttagcgctgctagaacatttaggggggaatctgggcgggttggtattagcagacttggccagaaatcctctattcaaactagaagtggtgtgacagtgggaggacgtgtgcattttcggtactctttaagcacaatagaagatgcaatagaagatgctggggaaatagaattacaggtcacaaatgggtcacaaggtcctagtgggtcactgcaacacacagcagaaacaattttaagtgagggccatgatgcatacgttgatgttgacatggatagtgtggggagtctttatagtgacattgatttgattgatgaacacagtgaaactcctcatggtattctggtgtttcatgatgaagctgaaacggatgtggtgcccgttatagatgtctcatatgtaaggaaaccattgtcaacaatacctggaagtgacctttggcctacaaatataaacatacagaatggtcccgtggatgtggatttgcaagacagcattttacctggcataatcatcacagattcaggggtggatggcacatattttttaaacacatacctacacccaagcttacacaaaagaaaaaaacgcaggttttcctgattgttttgcagatggcggtttggcagcagcaaggacaaagactatacttcccccctaaccctgtaactaaagtactgtgcacagaatcatatgtgaaaagaacatctatattttatcatggagaaacagaaagactactcacagtaggccacccttactggaaaattccagaacagaatattccaaaagttagtggcaatcagtatagagtctttagagtgcagctgccagaccctaatcaatttgcactgccagataaaaatttacataatcctgcaaaagaaagattggtgtgggcaatattgggccttcaggtaagtagaggacagcctctaggagcccctgtcacagggaatcaattgtttaatgtttggacagatgcagaaaatgtgactgcaaaaagagcactgccaggatcagatgatagaaagcagctaggaatggatgttaagcaaacacagatgctgctcataggctgcacacctgctattggtgaatattggggaaaagctattccttgtgaaggtaaacagccaaaagcaggggactgtcctcccatagaattaaagaacaagcctattgaggacggagatatgatggatataggctttggggcctgtgattggaaagacttcagtcaaaatttatctgatgtccccttagatcttattaattcaaaaagcttgtatccagattatttaaaaatggcagaggattctttaggtaacagctgctttttctatgcccggagagagcaagtgtatgttagacatgtttatagtagagggggtgaacaaaaagaagccatacctaaagacatgactctgccccagcaggtaccagataataaggacacttctttcacatttatggggacacctagtggctccttagtttctacagatggacagctatttaacagaccttactggctctaccaagctcaaggtttaaataacggggtctgctgggataatgagctatttattacagtaggagacaatagtagaggaggagtgtttacaattagtgttccagtggacgataggaaacctgagcagtacaatagtgccaatatgaatatttattgcaggcatgtagaggaatataagctagccgttattctggagctatgtagtgtggagctgacctcagaaaccgttgcatatttgcagaccgttaacccttctgtcttagaaaaatgggaagtaggagtgaaccctcccccagccactgtattagaagacacttatagatatcaggaatccaaggctataaaatgcatagatcagacggcagcagctaaaaaagataaatatgaaaatcttagcttttggaatattgatctcagagaaaaattatccgcagatttggatcaatatcctctaggcagacgcttcttagcacaaaatggtattacctgtagcagaaaaagattgcgccctgcaagtactaaaaaaagtacaacaaataagaaaagaaaaacatcacgttaaggtttgtattctggtatgtatgtactggggactgggagtgggcttcgggtgggtcaaatacttgcttgctaataaagctcttaatgatgcctatgtgtcatgaaatttgtgcaccacacccggtcgggttgctacaagtaggtataaaactggcgccaagtgctgggctctggccaccttggcgccagcactatctttttggccgaagatccactgcagctgcagctgcaaaactgcagcgtcgacagaaacaagcctctgcgcgccaaaaacgctgacggtgcccattgttaaagggtggattagagcagctgtaagggtgagtaacaccggaagccttgggagggctcctgcaccgctgccggtcttaaaggtgtcccccgagcgaccgacctctcgcggtcgaaattaaataggaaataactgaaaaaattagaccagcagcggtgcatggcgccgctttatgacatcggacacacgccctacgggataaattagggcgaagtagaacaataacccagtaaaaaggaagtaaaacggatatacaccgcgagcggtgttttggtactgtgtgcaaaggtaagtcaaggcgggaacctctcaaccaatcaattcaaactgtaccgggagcggtgccaccgcctgcggttttccttctttttctaggagaagattgttgttaacaatgagaatgcttatagtacagccaaaaactgaggatgctgggacaaaaaaaaaagtggtatataaagtgccgcaccgcaggaggttgtttattagatagtggggact

SEQ ID NO: 10 - DNA, 7353 bp, Human papillomavirus type 4
gtctgtaatgatagttggcaacaatcattacttatagctatatataaccggaagagatacatataaaaagggacagtgcatttctactaaatcctgtccagatggcagatggcagacctgcaaccttggacgacttctgcagacgattcgacatttccttttttgatttgcgccttacttgtattttttgttctcatactgtcgatcttgcggatcttgctttattctatcttaagaaacttagtttagtatttagaggaaattgttattatgcatgttgttctgaatgcttaagattaagtgcactgtttgaacaagagaattattttcaatgttctattaaagctgtacatttggaggaaattgctcagaaaaagattaaggaaatttgcattagatgcatttgctgccttagattacttgatattgttgagaaattagatttattatactctgacgagacttgctatttaataaggggtttgtggaggggctattgcagaaattgtattaggaaacaatgagaggagcagcgcccacggttgcagatcttaatttagaactaaatgacttagtgttaccagcaaacctgctgagtgaggaggtcttgcaatcttcagatgatgagtatgagattacagaggaggagtcggtggttccatttagaatagacacctgttgctatagatgtgaagttgctgtaagaattacattgtatgctgctgagctcggactacggaccttggaacaacttcttgtagaaggaaagctgacgttttgctgcaccgcttgtgcaagaagtcttaacagaaatggcagataaaggtacagacaattttgacttagaagggaataattggtatattgtccatgaagcagaatgcactgacagtatagatacgttggatgatttatgcgacgaaagtaatgacgattcaaacatttctaacttaattgatgacgatgtcgttgatcaggggaattcccttgcgctgtacaatgcacaaataaatgaggattgtgacaatgcactagcacacctaaaacgaaagtataacaaaagtccagagcaggcagtcgctgaattgagtccgcagttgcaggctgtgaaaataactcctgaaagacacagcaaaaggagattatttcaggacagtgggattttcgaagatgaagctgaaaattctcttacacaggtagaatccgagagccaggctggaccttctagccaagatggcggcggagatattaatttgttgttgttacaaagtagtaacaggagggcaacaatgctagcaaagtttaaagaatggtatggggtctcatacaatgaaataacaagaatttataaaagtgataaatcttgtagtgataattgggtaatagttatttttagagctgctgttgaagtattagaaagttcaaagattgttttaaagcagcattgtacatatattcaagttaagatctttggattttcagctttatatttagtacagtttaaaagtgcgaaaagtagagaaactgtacaaaagttgatgtgttctatattaaatatccaagaatatcaaatgttatgtgatcctccaaaattacgaagtgtacccacagcattatacttttataagcatgctatgttaacagagagttctgtttttggacaaacaccggattggatcgcaaaacaaactctcgtaagtcatcaagcagcaactactgcagagacttttgagttatctagaatggttcagtgggcatacgataataattatgtggatgaatgtgacattgcttatcactatgcaatgtacgcagaggaggatgcaaatgctgctgcttatttaaaaagtaataatcaagtaaagcatgtacgagattgtagtacaatggtcaggatgtataaaagatatgaaatgagagatatgtcaatgtcagaatggatttataaatgttgtgatgaatgttctgaagaaggagattggaagccaatctcacagtttttaaaatatcaaggtgttaatatattatcctttcttatagtgcttaaatcatttttaaaaggtattccaaaaaaaaactgtatagttattcatggtccaccagatacaggaaaatcattattttgttattcttttataaaatttttaaaaggaaaagtagtttcatatgtaaatagaagtagccatttttggttgcagcctctgatggattgcaaggtaggatttatggatgatgctacctatgtgtgctggacatatatagatcaaaatttaaggaatgcattagatggtaatccaatgtgtattgacgctaaacacagagcaccacaacaattaaaattaccaccaatgctaataacgtcaaatattgatattaaacaggaacaatctttaatgtatttacacagtagaatacagtgttttaattttcctaacaaaatgcctattttagatgatggtagtcctatgtatacatttactgacggtacttggaaatcttttttccaaaagcttggcagacaattagaattaacagatcctgaagaggaaaacaatggagtccctagtcgcacgtttcgatgcacttcaagaagcaattctgactcatattgagtcacaggagagcactttggaatcccaaatccaatattgggaaaatatcagaaaagaaaatgctataatgcattatgctcgaaaacaaggcctaaccaaattaggtctacaaccacttcctacactagcagtaactgaatacaatgcaaagcaagctattcagatacatttaactttgcaatcattgttaaaatctccctttgcatctgaacggtggacattgacagatgttagtgcagaactgataaatacctctccacaaaactgtttaaaaaagggaggttatgatgttgctgtgtggtttgataatgatagacagaatgcaatgctgtacacaaattgggactttttatattatcaagatatgaatgaacagtggcacaaagttaaaggtgaagtggattatgatggcttatactttacagaccatacgggagaaagagcttattttacattatttagctctgatgctcaaagatttagcagaactggactgtggactgtgcattttaaaacccaagttatttcctcccctattgttagctctacatactcctcctccttcgacactgaggaacaacagttacccgggccctccaccagctactccgaagttaccgagcaggcgagccctactcgaaggaggaaaccgaggaaatccgacgcgacctccaccacgtcccctgaaaccgagggagtacgactacgacgaagacgacgagaaggaaaatcagggcccgggtcaggagaaaccccccgcaaaagaagaagaggaggaggaagaggaggaggagagaccgaattgggatctgcaccatctcctgcagaagtggggagcagacatcgacaagttgaaagacaaggtctgtcgcgacttggactcttacaagcagaagctagggatccgcctatgatattgttaaagggcacagcaaattctttgaaatgttggagatatagaaaagttaactcaaattgctgcaacttcttattcatgagtactgtttggaactgggttggagattgctcacataatcatagtcgcatgcttattgcatttgatagcactgaccaaagagacgcttttgtaaaacacaacctttttcctaaactgtgtacatatacctacggctcattgaatagtttataaaatgcaaagcttgagtagaaggaaaagagattcagttccaaatctttatgcaaaatgtcaactgtctggcaattgcctacctgatgtaaaaaataaagtagaagctgatactcttgctgatcgtttgctgagatggttgggaagtgtaatatacctaggaggcttgggtattggtactgggagaggtagtggggggtcaactgggtataatccaattggagctccaagtagagtcacacctagtggtactttagtaaggcctacagtgcctgtggaaagtttgggaccctcagaaataatcccaatagatgcaatagacccaacaacatcttctgttgtgccattagaggatctgaccatcccagatgtcacagtagatagtggagatacaagaggaataggggagactactcttcagcctgcacaagtagatatttcaacatcacatgaccctatatcagatgtcactggtgctagcagccaccctacaatcatatctggcgaggataacgccattgcagtgttagatgtgtcccctatagaacctcccacaaaacggatagcattggcaactaggggagcctcagcaactccacatgtaagtgtcatatctggcacaaccgaattcggtcagtcatctgatctgaatgtatttgtgaatgccacattttcaggcgattccattggttatacagaagaaattccattagaaccgttgaacccctttcaagaattcgaaatagaaagccctccaaaaactagtacaccacgtgacgttttaaatcgtgcaataggaagagcacgggatttatataatagaagggttcagcaaatacctactaggaacccagctttactgacacagccttcccgcgcaatagtatttggatttgaaaatcccgcctttgatgctgacatcactcaaacatttgagcgggatttagaacaggttgcagcagctccagatgctgactttgcagacatagtcactatagggcgtccaaggttttcagagacagatgctggtcaaattagagttagcaggcttggacgccgaggcacaataaaaactagaagtggtgtgcaaattgggcaggcggttcatttttattacgacctaagtacaatagatactgctgatgctattgaattatctactttaggtcaacattcaggagaacaaagcattgttgatgctatgatagaaagcagcttaatagatccttttgaaatgcccgatcctacttttacagaagaacaacagcttttagatccacttacagaagattttagtcagtcacacttggtgcttactagtagcagacgtgggacatcatttactatacctacaataccacctggattaggtcttagaatttatgtagatgatgtaggttctgatttatttgtttcctatccagaatctagagtaatacctgctggaggtttaccaactgagccatttgttcctctagaaccagctttgttatctgatatatttagtacggattttgtatatcgtcctagtttatatcgcaagaaacggaaacgattagaaatgttttaattgttttgcaggaacatgtcgagttggttatctacaacgggtaaagtctacttacctccagctcaacctgtggcaagagttttggaaactgacgaatatatcactggaacatctctgtatttccacgctggtacagaaaggcttttaactgtaggccatccttattttccagtgaaagatgtacaggaacctcacaaagtattagttcctaaggtttcaggaagtcaatttagagtgtttagattcaatttgccagacccaaacagatttgctttaattgataatggcttttatgattctgatcatgaacgcctagtatggaaactgaggggaatagaaataggaagaggaggaccgcttggtataggtactacaggtcatcctttatataataagtttggagacacagaaaatcctaatggctacaaaaagcaatcagatgataatagacaggatgtctctttagacccaaaacaaacacagatgtttattataggttgcactcctgcaataggtgaacattgggataaagctgaaccttgtcccagccctgctccgcaacagggagattgcccaccaatagagcttgtaaattcatacattcaagatggagatatgtgtgacattggatttggggctttcaattttaaagctttgcaggctgataaatctagtgctcctttggatgtcattgccacagtttgtaaatggccagattttttaaaaatggggaaagatatctatggagatagcttgtttttctttggaagaagagaacaactatatgccagacatttctttgtcagagcaggcaccatgggagatgctctaccagaaccttttgaagctacctcagattattttattggtgctcaaaaccaacaagatcagtacactttaggacctcatatttatgtagggacccctagtggctctttagtatccagtgaatcccagttgtttaatcgaccgtattggttaaacagagctcagggtacaaataatggaatttgttgggataatcagttgtttgttactcttgtagataacactcataatacaaactttacaatttctgtgaagtcagatggtgctaatgacaattatcagtataaagctagtgattttaaacagtacctcagacatatagaggagtttgaaatggaatttatatttcaactttgtaaagttcctctaactgcagatgttatggctcatttaaatgtaatgaatcctaatattttggataattggcagttaaattttgttccaccacctccctctggaattgaggatcaatatagatttttgcaatctagagctacaagatgccctacacagacccctgcaactgaaaaagaagatccatataaagatttgtctttttgggttgttgatttaagtgaaagattttccagtgaattgagccaattttccttaggcaggcggtttttatatcaaagtggtttaattaatggttctctaaaacgtaaaagaataataagttcttctcatgcacaaactaataccaaacgttctgccaaacgaaaacggtctctgaaataacaatgtgaactcttctggaatgttttattctgccaggaaaaccttcaactgagccaaattattatataatcgttcttaatctcaaaattgagctaattatataagatttgcaaacgtgtatgtatctgtttttgtgaactatagtgaaataaactgccacatacttgccagtgtccagtctctctgagtcatttggtcaacatgcgtccgcaccccaataattatttgcatacacagatcagtaggagaggcgccaagacggacatatcctcttcaaatttccttaaaattattgaatttaacaactgtaagctacaaaagaccgttatcgtttcctctaaccttgggaaaaaggtgagtgaaagttttattgcaccttttgtgagtcaatttgtctggcggcgctgaacgaatttggctgtcagcctttgcaccgggagtggtggaaaatagtttct

SEQ ID NO: 11 - DNA, 7687 bp, Mastomys natalensis papillomavirus
gcaacaatctcctctccatacttttttccactgcaccggtatcgtacaaacatatataagaagaccccattggctatggatttgttactgcggtagccggatggataggaccgtgcactcctttgtggagcggctgggaattcctcgggaggacctcctgctgccgtgcacattctgctcgaggtttcttacccaggaggaattaactgcatttgactttagtgcttttaaccttgtttggagaggaaggtgtgcccatggaatctgcacagcctgtgctcgtgtctgtgcatccctagacctgtttctgcaccatcagaattcgcgaccattagcagatgttctgcgggacgaaaatcttacactccacggactgaaagcacggtgtcgcgtgtgcatgaagatactgtcagtgacagaaaagctagagtgtgcagaaagaggggaatcctttgccaaagtcaggggccagtggagggcacggtgcagaatttgcaaacccgtgtaaaatgataggacctgacaccacgcgctgtctcaccggcgaaactcctgactcggtcagcctgtattgtcacgaagttctcgacgaggacgaattaaaagagccaacagaggcggctccgccaccggaacaatacaccttgtaccaggtactcattgagtgtcctgagtgtaataagacaattcggctgacgtgcgcggcacaagcacaccagatccgtgggctagaacatctactgcttgacgggctaagagtgatctgtccgcggtgtaaccagaagaatggaagatcttgaagaaggtactggcgaaggttgcagtggctggtttgatagagaagctatttgtagtgacgggtctagtgatgaggagccaaatgagtcctttgaatctattgcggacatgttcgatgacggaacacaaacacagggcaattccctagagttgttccatacccaggaaaaggaggagactaggacacagatacaagctctaaagcgaaagtacattcccagtccagaggcaggtggggatctctcaccacggctgagggctatctctattacccccaaaaaaaagaaacctagcagacggttgtttgagaccccagaggatagcggcaacgggagtcttgggaatgagactacagatacttcttcggggtttcaggtagtaggggactcagctgtggatgtatgcgatgcggggcggctgcttaatctgaatctgcttcaaagccataatagggtggcgaggttgcttgctgtcttcaaggaagcttatggggtgtcatacaaagagcttacacgggagtacaagagcgataaaacctgcaatccagattgggttatcgcattgtactccttgagtgagcccatcctaaatgcggcgcggacaacactacagggaatttgtgagtatgtgtttatgcaaagccgccctacagcggcagccacagttgctttactaactgttcgctttaaatgcagtaaaagcagggagacagtaagaaaacaaatgtgcggcatgttccactcagatccgctactctgcctgtgtgatccccccaaggtccaaagtgtgcctgcagctctatactggtataagagcagcatgtatagtgggacattcacacacggagaggcgcctgagtggatcaagagacagaccatgattacctgtgcaatggaagagactaaatttgacctttcagaaatggtgcagtgggcatatgacaataactatgaggacgaatcccaaatagcatttgaatatgctagaacagccactgagagccctaatgcgaatgcctggctggcttccaatgcacaagctaaacatgtgagggactgtgctacaatggtgaggcattataaacgggcggagatgaaggctatgagtatgtcacagtgggtatggaagtgctgtagagaggaacctgaggagggcacttggacacctatttccctatatctcgcgtccgaaggggtggaagtgataagatttctatctgctatgaagagttggttgcgggggattccaaagaaaaattgtctggtattttacggccctccaaatacagggaagagtctgtttactatgagccttattaagtttttgagagggcgagttatatcatttgccaatagcaaaagccatttctggatgcagccactggctgaggcaaaggtagtgcttttagatgatgccacaagggccacatgggactatgtagatacatatatgaggaatgccatggatggaaatccattatcaattgattgcaagtatagaacacctgtgcaggtaaaatgcccccccatgcttgtcacaacaaatgaggatgtgcacttgaatgataggtggcgctaccttcatagcagaatacaagtctttcacttaaaggaacctatgcctatagacactgccggtaacccagagtattccttctctaatagacattggaaggcgtttttcgaaaagttacagaagccactagatctaagcgaggacgagggtgaccccaaggacaatggagagcatacacagccgtttagctgctgtgcaagaggaactgatgtgcatgtatgaggatggggaggagacactggaggcccagcttaaacattggggcttgttgaggaaagagcaagtcttgttacatgcagcacgccagcatggacataacaaaataggactgcaggccgtgccccctctttcagtgacccagcagaatgccaagaatgctattgaaatgcatttgctgttgcaaagtcttgcagagacaccatatgctagggaagcatggacactaagccagaccagcagggaaatgtatatggcaggtccatccggcaccttcaagaaagacggcaccattgtggaggttatatttgatggtgacaagactaatatgatgacatatacaaagtgggggaagatatactttgctgatccaaatggcaattggagcagaacaacctcccatacggacattaatggcatatattttaataagtctggggataaggagtactatgtgcggttcaaagaggaagcaaagaggtactcattaacaggaacttgggaagtacatgatggactagagacacattcccttcttattcctgtcaccagctctacaccgcagaccggatttcctagaggggatccggtacgccttcacgggaataccaccacaggactgcccataccgcttcggaacagcagcagcaaccagatattactacgagagggaagaggagactatccagacggcgcacgccgcgagacgaggaggtactaccaggggccaacaccgacgcccaggtctctatctccccccatctaccgtcccccgccaagctacgaagagtcgaggaggaggaggaagctaaggcgccgccaagacgggcgagtcaaataccccgcgtctccctacaggacaaaaccaccgggggaaaccagcagcgacgacgaagacgaggggagaggggggcacgaaccccgtccccagagacgactgcccagaggcctaagagaccgcggagagcgtgcacccgaaaggaggagacccccagttcaggagggggaggaggacgtggacggcgtaggggccttgctggacgacctgaagctgtaccaagaaccacctggagacccagtggaggactcggactccccaggcagtcgtcttacccccgccccgccagacctatctcggtacgactctacccggttacaggtggacgcggagagcagccctcctaggacacccagaccggcccccactctcgtggcagagtgcactcctgggagaccttctccgcagactggaagcggacagcaagcactgggagaaccgccttctcggccttcacgcggacattgccgcgaccctcggactgcctgccttttgatcatcaaaggatcatcaaatcaggttaagtgcttgcgatttagacttaaatcctggcatcacagcctgttttcctacatcagcaccacatggcagtgggttccttcagtaggaagtaataggattggacggtcacgcattctggtgatgtgtgaggactcagcgcagatggacagattcctatgtactgttaagatccctgctggtatgacagttgaacagtgcagcatggcgtctgtctgatgcccccccccctcgcataacatactaacgcacactgcaataaagtttttcctttacacagtactaacctactaatattagcatgtctagaaggagaaagcgacatacacgagtccctcgtgactcggccactcacatatatcaaacatgtaagcaggcaggcacatgtccgcctgatgtagttaataaagttgaaggcacaaccacagctgataagattcttcaatatggcggggcggctgtattcctcggtggccttggtattggtacaggtaggggaagtggtggtgcaacagggtatgtaccggtcggcgagacacctggtatttccgtgggtgcaagaccagttcctcgacctaatgtgcccttagaaactgttggtccccaggacctgtttcctgtggatgccattaggcctactgatccttcggtgattgatgtcgccagtgtgcctactcccactgacacctctattaatgtacccgaggtggaggtcattgctgagatccaccctgtacctcctgacggtccctccaacacaccaacaaccacaattaacacatcaggctcaggggatgcagccatattagaggtagctcctgaaccatccccagccgtcaggactcggtggagagctagcaagacaaccttccataatcctgcctttcacagcttctcctctactggttcaactgtaggcgaggccacaggtatggacaatattgttgtttacagcggtagtggggggaggacgataggtggggacagcatagagcttatgccctttactagcagtgataccctagatttaagtattgtggaggagacctcctttggaggtaggaccagcacaccacgaaccaagcccctcccttctcggttgccttcccggaggtattatgaatatagagaaagcagtcttggtgagttatggtcacctaggagggctatgggtcccacgtatataaatcctgcctttgaagctgaggatagtatcctttttcctgaatgtagcatgcaggccgctaatccagattacacaggcattaccaggcttggtcatctctttggtactgagcagggtggccgtgtccgtattggtcgtctgggacaaaagacatccctgcacacacgcagcggtatggcaataggccctaaggcatacttttataaggacatttctagcatttctgttgtcccagaggagagtatagaactcagcacctatacctcagctgcccctttgggtgaggatgcaggtataatagtggaggactctatggagggttcttttgacaatatcaccctcagttcttggagtcatggatccatggacgggcttcttgaggatgatgctagttatgattttcacggccacctggtgtggggaacacgccgtagctctaagcaaataagcatgccattccgccggtcgtggtatcctgaaactgctgtgtacgtgcaggagggtgggtctgtaatggatcctgaggcttctgcagagctggttcccagtagggacagtgctcgtccccatgtcatatataggggctataatgggacggactattatctacacccgtcattgtccagacgcaggcgtaacagcaggcatatctatttttcagatggcgtactggctgcctaataaccagaagttgtacctgcccccggccccggtgcagcgcatactgtctacagatgaatttactacacgaacagacatatattactatgctagtagtgacaggttattaactgttggtaatccatattatcctatactggatggggatactgttactgttcctaaggtcagtcctaatcaatacagggtgttccgttgtaaattaccggaccctaaccggtttgcatttggtgagaagtcggtttacgaccctgagaagcaacggcttgcatggtgtatacggggagtggagatagctcgtggccaacctctgggaatagggattactgggcatcccctatataacaggctagaggatgtggagaaccctggaaagtatccatctgctccgggcacggacaatagacaaaatgtaggccttgatccgaagcagactcagatgtttattgtcggttgtgtacctgcacagggtgagcactggagtagagcacttacctgcagcaatcaggtggttaagaagggtgactgtccacctattcagcggatgtctgggatgattgaggatggtgacatgggggacataggttatggcaacttagacttccgagtgttgcaggaaaacaagtcagaggttcccctcgaggtagttgactctatctgtaagtaccccgattatttaggaatgtccaaggaaacccacggcaactcatgcttcttctatgctaggcaggcgagattatacagcaggcacttctttaaccgtgcaggtgttcagggtgagactgtgccggagtcattatacaagaagggcaaggatggacaggcacagagcacactggcactagctacatactcagggactccgtcagggtcactagtgtcatctgatgctgtactgttcaaccgtccatactggcttgagagggcacaaggacaaaacaatggcattctgtggaataatgatttgttcgtgaccgtgctggacaacactcgtgggacccatttctccatcagcattgctacacaggatgaaaatgattacaccgcctcaaactacaagcaatatactcgacatgttgaagaatttgagcttgaatttattttccaactggttaagatcaacctttctactgaggtgctagcatacctgcatgggatggacccatctatactggataactggaacttgactctgggaccccccaatgatggtagccttgctgataagtacagatttatagaatcccttgctacaaaatgccctgacaatgtggaagtcactaagcctgatccctacaaaggacggatattctggaacattgacctgactgaaagactgacagctgatctggaccaattctcacttggacggaagttcctctaccagcacgcgcgaatttcaaaccgtaaacggtcccttcctgcttccagaaacggcggcggaacctcctcctcttccaccaagcggagaaaaaaatagttggaataaagactgctgacactgcacttgtgtcccgccttttcttaatcccgcctttgctggggctgcagtacagcacgctgccaagtttatgggaggtgctggaacactgggcggtgcttggatccggaatgcgccgccttggaagccagcgccagtcttgttcagacaccgagacgccaggtgtgcagcttcattggtgcaccgtgccaggtatacctctttcggtgcagttcttatgccaagtctattgttgctttttgccaactcggtgagtaacatcctgcttggcactgtctgcgacgtacctgctgcagagacttgtaccgggtgcggtacttggcagtacaaacacaattaggtttggacaagaccgatatgggtgtgaatgttg

SEQ ID NO: 12 - DNA, 7565 bp, Rabbit oral papillomavirus
ggcaacaacaattgacaggagaatttagaagagaaacaacctttatcggtctgccaacttttagggtataaataagtagtttctacggaaattttcaaatggaggagccacgcaccttggtggagctttgcgaccgtctaaattgtacctttgaaagcttgaatattccttgtatcttttgtagtgcgccgctgtctttgggggataaagcattgtttgattttagtcagctaaaacttgtatggagggacgggtggcctcatggcagctgcagagtatgttgtagatatttttgttatatggatttggtttttaacctggattatattaattcttggagtcaaattgaagcgctttttaatcaggacattgcagaatggttcatgcgttgtacatgttgtgcaagagtgctaactacgtctgataagattgatctgaaatctgagaatagagatttatttagaattgtttcatattcgggaaccgtgcagtggagagcattgtgcggtccttgcagacggggggtggggacattgcctcaaatacctttgcttgaaaggcctttgccaacacagcctttgtgcaactgcagcttttgcagcccattgcttgcagaagcagcacctttagttactgtagattctggaggtggttccagatcagcgattcctactgggccttgcgactgcgaggagtgcagtggtaataggacacccgacggggaggaggaaccaggagcaagaaatacaagtagccctgacagttctgtttgtggttgtcctctttgcgagggagaaattattaacgtttaagcttcttgctcatatttggggcgtaggtataataaagcaaaaattatgataggccccaagcctacccttcaggacattgtgcttacagaaactgctgatcctgtaaatctgcattgcaatgagcgtgtggatgatataattctggaggaggaggatcagcagggcaaacaggtgcattcagcttatcttatagtagtagagtgtggggactgcggcaagaggctacgctttacctgtgtgagcgacaaggactccattcggggctttcaacaactactattgggaccgctgctgttactttgcaaggactgcgccgcaaacgtctgaaaaatggctgaaggtacagaccctttagagggtagcagtgggtggtttttagtgactgaagcagactgtgtggatggagtaccggactttgacgatgagtttgaggaactgtttgaaggtgacactataggagatttaatcgacaatacagatcaggctcagggaaattccctggaattgttccaccaacaggaaactgctgaggttttagcggagatagctcagcttaagagaaagtattgtgatagccccgagaaaaacaaaactgaaactgaacgaaatgtaaatgcactcagtccacgcatgcagtctgtctcgttaacaggcaagaaagattctgtgaaaaaaagactatttggattgggaaatgaagctgtatctcttaatggcgcgtcacagcaggtagaatcgggattggggacgggacaaagtttaaattctagcgcgcgggagaccattactgattctttgtcttccgttactagtgtttttacccaatcttcttcaaggatagctcagctagcgatttttaaggaagcccatacagtaagttttgctgaactaactagaccctttaaaagcgataagactgtatgcggagactgggttgctgggatatcgggagtgcattgtgccttgggtgacagcttaaaaacctccttaagaagccattgcatgttttttttgtatgatttaaactacgctgctaataattccacatctatactgcttttgctaagatttaaatcacaaaagtcgcgcgatacagtgacaagtttgctaaaacagcttttaggggttgaccacattcaggtcatgttggatcctccaaagaccagaagcgttcctgcggctttgttttggtacaagcgagccatggtaacagcagttgaaagctttggtccatttccagaatggatcactcagcaaacacaagtaaatcatcaaatggcgcaggaaaaacagtttgagctgtccaccatgatacagtgggcttatgacaatcacataacagaagagagcaaaattgcttatcaatatgctttgcttgcagactcagatgagaatgccaaagcgtttttagcctcaaatgctcaggcaaaatatgtaaaagactgtgctgctatggttcgcttgtatttcagagccgaaatgcaggaaatgtctatatctgcatggatacattataggctgcaaaaggttgatggtgcgggggactggaaggaaatagtaaggtttttgagatttcaaggaattgaatatattccattcatgatttcaatgaaaaaatttttgaaagggacacctaaaaagaattgcattgtgatttacggtccacctaatagtggcaaatcatatttttgcatgagcctgctaagactgatgggagggaaggtaatttcctttgcaaatagtaaaagccatttttggctacagccactagcagatgctaaaatagggctgttggacgatgctacaaagccctgttgggattttattgatacatatttaagaaatgctttggatggcaatccaatatcagttgattgcaagcacagggctccaacagaacttaaatgcccaccattattagttaccactaatgtagatgtgatgggtgatgacagatggatgtacttacatagtagaatagtgtttttacgcttcatgaataaaatgccattaaaaagtgatggtacacctggttacaaccttgacgataaaaactggaaatctttctttacaaggttctgggaaaccttagagctcagtgatcaagaagaggagggggacgatggaaaccctcagccaacgcttagactctgtgcaagaacagcttctcaatctttatgaaaaggagagccaaagcttgcaagatcaaattgcacactggaatttgatcaggaaagagcaagttatattgcactttgccagaaaacatggcataaggcgcttgggaatgacatatgtgcctaccttggctgctacacagcacaatgctaaacaggctattgaaattgtattgtttttggagagtttacagaggagtcagtatggtcaggaaccgtggacattgcaggacacaagcaaggagagatttaaaagccctccttctaattgttttaaaaagggggctcagaccgtggaggtgatttatgatggtaacaaggataataattttagatataccgtgtggaagtttatatatttttgggatgaaagcggtgattggcataaggtacctagcactgttgatgagaagggggtgtattatagggatacagaaggaaacaatatatattatgtggactttgagactgatgctgcacgcttttcaagcaaaggagagtatgaagttgtatataaaagccaaaaactttctgtgtcctctgttaccagctcaacccccctacggcccatcgctcttggcaacacccctgacaacgccaccgcgtcgcccgcccctgcagtatccgcaggccccgcgcaccatccgcaaaccccggtcaagtcgttatcggggccggtttctcgttacggacggaggagatccagatccccaggagttggattcgacccagcaagatccagaagacaaggaaaacatcccaccgacttcaacgccaacaccatctccgccgactccaccgactccaccgacttctcgcccgcctttagaccacctactccttcagaggttggaagaagaaatacgacagctccaagagagtctgcaagaggacttggaggaagagttcggcaacttatatctgaggctcgggatccgccagtgatctgtttaaaagggggcaacaaccagcttaagtgcttaaggtatcgcttaaaagccaaacaccgcactttgtttgactgtattagtaccacatggagttgggtagataatagcagcacatgcagagtaggtagtgggagagtgctgataaaattcaaagatgaagcacaacgtgaaaggtttttagaggaggtaccgatacccagacatatgcaagtgtttgttgggaacttctttggcttgtaaatgcatttctgtgaattatctgtatctgtgaaatttgtacattgtttacaatggtgctacgcacgcgcaagcgcagagctgctccacaagacatttatcctgcatgcaaaatatctaacacatgtcccccagacattattaataaatatgaaaataaaacagtggctgacaagattttacagtatgggagtcttggagtgtactttgggggtttgggaataggtactggaactgggtcgggtggcaggggtggttatgttcccttgggaggctcatcaggcggacgggtagtaggtggctctgctgtaagaccacctatccctacagacactgtaggtcctttagaagtaatccctgaagcggttgaccccgcagggtcttcaatcgttccccttgaagaatatcctgctgaaataccaacaacaagtggcactaatgtcataggtgaaggaggtgcccagcccccacccagttcagggggcggcagcgcaatcctggacgtaatcagcgaggaaagtggagtcacaagcagaacacactttaataaccccacctttgaagcccccaatacaaataatattagtgtccctgacattgtagacccccaaccagaagacatagttattagctacacagatgccccagaacctggtgagctcatagaattggtacccttgcatccacggggaagagaaacatttgacatacaagaggaaacctcattcattacaagtacccctgaccccgcctccagccaggcggcgcgaactgcaaaccttgccagtaggcggtaccaacaaattcaagtgagcgacccgctctttttaggacaaccccgtaagctggtgcagtttgaaaacacttttgaaaatcccgccttcgtagatgatgatcagttaactcttttgtttgatcaggaccttgataatgtccttgctgcaccagacccccaattcactgacgtggtcaaactgtccagaccctcttatacaagaacggcctcaggtcgagtgagagtcagcagacttggtactactggcactatccgcacacgcagtggtctgcaaataggcccccgcaagcacttttattatgatatctcatcgataccatctgaaagtatagagctacaacccattgcagaatctgcaaatgaagacacagttagtgggctgcctgacctagacatcatcaatgcagatgaaactgcatttactgaggctgaccttttggatgagccagaatctgtgggcgaaggcctgcagctggtgattagttccactagacgggcaccacggatcctaccgatgcccaagttatttgccactgatgtccatccaggctttttcccagacatacacatagattacaaccagccagatgtattacctggttttgaagagggaactattacccccagcttttcatttaacaatagcggtgactttgtacttcacccttctctaagacgccgtagaaaacgcaaatttgttttttaattgcagatggcggtgtggctgtcccagcagagtaaattttatgtgccaccacaacccattacaaagattctcagcacggatgaatatgttagcagaacaaacatcttttatcatgcatcaacagaccgcctgttaacagtgggacatccgtattatgagctagaaaaaggtggaacggtggtagtaccaaaggtgtcacctaatcaatacagagttttccgtgtaaggctgcctgatcctaataaatttgcttttaatgataagcaattgtatgaccctgaaaaggaacgtttagtatgggctgtaagaggtgtagaagtgggaaggggtcagccgcttggtgtaaatgtcactggtaaccctcttttcaatcgctatgatgacgttgaaaattctagtagatacaacagtggacacaataatgaccaagacaacaggcaaaacattgcttttgatccaaagcaaacacagttgtttatacttggttgtgtcccggcaaccggtgagcactggacccaagcgcagcgttgcgctggagctggttatgagcaaggggattgtcccccaatagaactaatcaatacagtgattgaagacggggatatgagtgacataggcttgggagctatggatcatagactgctgcaggtcagtaaggcagaagtaccaatggaacttgtaaactcagttagtaaatatccggattatattaaaatgctcaaagacccatttggggacagtttgtttttctatgcacgaggggagcaaatgtatgctcgacattttttcagtagagctggggatgataaggaaaaccccaccgataccttgataacaggtaaaggaaatcagtccacagtatccactgacaactatatggtcactcctagtgggtcattggtgtccagtgattcacaagtatttaatagggcttactggctacaacgagctcagggtatgaataatggcatttgctggaacaatcaaatgtttgtgaccatagtggacaacactcgtggcactgtcatgaacattgtcacaaaggcgaatggcaatggcgcagttgatacttgggctaacaatgctttcaagtcttatctgcgacatgtggaagagtttgagctgcagttcattgtacagctttgcaaagtccgtctcagtccagaaaaccttgcatttttgcacaaaatgcaaccatcaataatcgataattggcaattatcaataaccgcccccgcaacttccaatttggaggatcaatacagatttattcagtccttagcaacaaaatgtccaccggttgaacccccacaagaagacacagacccctataaaaactataaattctgggatgttgacctctctgaaaaaatgtcagatcagctagatcaatttcccttgggaagaaaatttttgaaccaaagtgggctggggcaaaatagacaagttaaaacggctgctcccactacaagtatgcgaggattaaagagaaaaagacgtatttaatgtatttctgccgtatgtaatgtttatgttaaaaataaatactgacatgtgtcaaaaggctaaaaaggattgtctgattttatctcgaccgcgcccggtgacacactccgtaaggacgaggaacaaaagaaatccaaatcggtgacaagctcagacggtcttccggattagcacaaccgcctgagctctcgggcgatccttttagtttttggcagaacatcgtgttttcttgaagattagcgcggttactcagcaattttacagttttgtcttgtctggagaccgctgccggtcgcgctaatccctcaggtaagcttaaaggattagctgctcgaccgaaagcggtcccaggtggtaggaattactcaggcgattgttgtt

SEQ ID NO: 13 - DNA, 8607 bp, Canine oral papillomavirus
aaaaggtgtgttctcttattgtagctaacaacaatcttacttacagtaaaattccaagaccgatttcggtcctggcaactgtttcggtcggtatatatagcatgttttggggggcactgttatcaatggagcgcccgacgtcggtgagagatctttgcatgtctctaaagctctctttgcttgatctgtcgcttgcttgcaaattttgtggcaataatataacaaatatagaaaagctgctttttgataaagctggttttcagttaatctggcgagaaaacaacgcatttggatgctgtcagtactgtgcaagagtctgcagcgttgtggagcaatgttttggaagccacagacacttgacttctgaggagcttgtcaacgtaacaaagaccttgcagcagcttagtcttagatgtttaggatgcctcagtattctgagtgaggcggacaaagaactatgtgctgaattgaacgatttttctgtggtcagggggaaaactaggggcttgtgttcgctgtgccgattaccaccatgattgggcaatgcgcaacccttttggatattgtgctgacagagcagccggagccgatagacttgcaatgctatgaacaattaccatcgtctgacgaggaggaggaggaggaggagccaactgaaaaaaatgtttacagaatagaggctgcctgtggattttgtgggaaaggggtgaggtttttttgtctgtctcaaaaagaggatctgcgtgtgctgcaggtcactttgctgagcctcagcctggtgtgcaccacctgtgtgcagaccgccaagcttgaccatggcggctagaaaaggtactgactctgagactgaggatggtggttgggtactaatagaggcagattgtagtgaggtagactctgcagatgaaaccagtgaaaatgcaagtaatgtctctgatcttgtagacaatgcgagcattgcagaaacacagggactttccctgcaactgtttcaacagcaagagctgactgaatgtgaagagcagttgcagcagctaaaacgaaagtttgtacaaagcccgcaatctcgggatttgtgtagccttagtccgcaattggcaagcattagcttaacgccacggacgtctaaaaaggttaaaaagcagctgtttgcaactgatagtggcattcagtccagcaatgaagctgatgattctcttgaggggcaaagacaggtagaaccgttgccgggtcgggaagaaaatggcgccgatgcattgtttaaagtgagggataagcgcgcctttttgtattcaaaatttaaatcttcgtttggaataagctttacagatttaactagagtttataatagtgataaaacttgcagctcggattgggtagtatgtctttatcatgtatctgatgatagaagagaggcaggaaaaacattattgcaggatcattgtgaatatttttttttgcattcaatggggttttgtactttgttattattatgtttgtttgtgcctaagtgtagaaatactttgtttaaattatgtagaagtttatttcatataagtaatgtacagatgttggctgatcctcctaaaactagaagtcctgcagttgcattatattggtataaaaaagggtttgcatcaggtacatttacacacggagagttgccaagttggatagctcagcagacactaataacacatcatttagctgcagagaaaacctttgatttgagtgagatggttcagtgggcttatgataatgatctgaaagacgagtctgaaattgcatacaaatatgcagcattagcagaaacagatgaaaatgctttagcttttttaaagtctaataaccagcctaaacatgtaaaagactgtgcaacaatgtgcagatattataaaaaagctgaaatgaaaagattaagtatgtctcagtggatagacgaaagatgcaaggctactgatgatggtccaggtgattggaaggaagttgtgaaatttttaagacatcaagggatagaatttattttgtttttggcagactttaaaagatttttgagaggtaggcctaaaaaaaattgccttgtattctggggtcctccaaatacaggcaagtctatgttttgcatgagcctgcttagttttttgcacggagtagttatttcatatgtcaatagcaaaagtcatttttggctgcaacctcttacagaggggaaaatgggtctgttagatgatgccactaggccttgctggctctatatagacacttatttgagaaatgctctagatggcaatacatttagtgttgattgcaagcacaaagcgcctttgcaactaaaatgcccgcctctgctgattactactaatgtcaatgtttgtggagatgaaaaatttaaatatcttcgcagcagatgctctttctttcattttccacaagaatttcctttggatgacaatggaaatcctggctttcagttaaatgaccaaagctgggcttctttttttaaaaggttctggaaacatttagatttaagtgaccctgaagacggggaagatggagaaactcagcgaggccttagacttactgcaagaggaactactgagtctgtatgagcagaatagccaaagtcttgcagaccaatcaaggcactggtcattgctcagaaaagagcaagtcctactttattatgccagaggcaagggcataatgaggataggcatgcagcctgtgcctccacagtctgtgtctcaagccaaagctaagcaggccatagagcagtcactttacatagacagcttgttacactcaaagtatgcaaatgaaccgtggacactatgcgatacaagcagggagaggttggttgcagaacctgcatacaccttcaaaaaaggtggaaagcagattgatgtcagatatggtgacagtgaggaaaacattgtcagatatgtattgtggctggatatctattaccaggatgagtttgacacctgggaaaaagcacatggcaagctagatcacaaaggactctcatacatgcatgggactcagcaggtgtattatgtggactttgaagaggaggccaacaaatatagcgagactgggaaatatgagattctaaaccaacccactactattcccaccaccagtgccgccggaacctccggaccggaactccccggtcactccgcctcggggtccggtgcctgttcccttacccccaggaaagggccgtcacggcggcctggacggaggtcgtcgcggttccccagaaggtcaggaggacgaggaagactcggacgaggaggaagcggagaattacccccccagccgcagccgtcctcgtcgtggtcgccgccgtctccacaacaagtgggatcaaaacatcaactacgaaccaccagcagcgccggaggacgactgggaagacttctgcaagaagcttacgatcccccagttcttgttttagctggggatcctaatagtttaaaatgcataagatatagattaagtcataagcatagggggttatatttgggggccagcacgacgtggaaatggacatcaggcggggatggagcatctaagcatgaccggggcagtgcgcggatgctgttagcatttttaagtgatcaacaacgggaggactttatggacagagtgacttttcctaagtctgtgcgagtatttcggggagggttagatgagttataagggagggagggggggatggtgtggtgaaggggaccaaaaaaaaaaggttaacaggtgtagtaaggtgttgggacatataactgaaagggtggttcataagaatttcagccagagctttaatatttacagagtcaacaaacgttagtctcttagcctaaaaatttaagcagtttaaagcataagcaaacaatctttcattatttttctttttttttttttttttttagtgtgtagacttcacagaagagggcaaaacaaaaacattgtgtgtagtgttagataacagtcatattttagctaccatagtcttatttttttttctctgttttgttttaagtcaccatttattcactgcgaaaataccatcatatattcttagcctagctatattcatcagttttagaccattatccattagttgctaagttagtgttaggtaacaacgcttaggtaaacaacagaaaagatcatagtgtttacaacaacaacaagatagttaaagtgaacaacgacaagtccgacagtccaaccctactttgtgtttatttgtctctcgtatagtataaggcatatagtatcagttaagttagaaaatgtgtaaataagccagatatatcgatatccaattaggcaagtgaaacttcaatatgtttattgcagtctccaaatttagtttagtttgatatgttgtctgattagattatattgcagttgggagaatatcaagaacacaccatccacagacaaatgcattgacatcggaacaaaaagaaagaaaagaagtagaaaatgggaatgaaagtttgaatgaatgctgttaattttgttccatttgtacctgttatagctatctcttttttggtcgatcctttgtgatcctctgtacatactgtagaaaaccagtggaattcagaaactctactacttataagctgtcccccccacctgctctttcatcatactgtaaaactgtttactgttcaactacaatactgttatactgttttctgggcaccttctctctcactagcaaaacaatggttcactgaaaaatccagtatattgtaaatattgtatatacaaagttttgaaattgttcacaactgtataacctgtagaacatctctgcatttcttcacatgtcttacataggataacacacatttgcaaattgttctgagttgttgcatactccataagttccttttgaagttcctcttttttttttttgtagaaatccttaagtttttttatattgtaccggcagtcacagtaggtgtttgaatctagaataactacatcatacataccatcaaatcaaaccatcagtgaaacatcatcagctttaatgtcaaaagcaatcatagcattagcactgttgcataagtccaaaaactactcgacccccctacccgcttatctgaccaagagtctgtgtgcggacatctgagcacattgtaaatacatttaaccgctaaaaccccgtgcagcaaaggaaagacaaaaaaaagtccccgctgtcacaaccgtgacagcaaagaaagacaaaaaaaaataaaaaacatccctcgcttacgcaatggcattgatcaggaaaagacgcgcagcccctcaagatatataccctgcttgtaaagtgtccaacacttgccccgctgatattttgaataaaatggagcaaaatacgcttgcagataaaatcctcaaatatggtagtgctggtgtttttttggggggtctaggaatatcaacaggcaaaggggtaggggggcgcacaggttacattcctttgggaggaacagaaagtggagtgggtgtaggcacaagggtcacaacaataagacctactgtcccaataagcagtgtggggtctcctgactttattcctgtagatgcagtagaccctctagggcctgcagtcatacccccagaaagatttcctatagcagtagaggatccttttactctacctccaccacgtttcccaactgcagtagaagaagatgtaattgagctgcagcctattccaggcccctcatctgaaatcccactcgctggccctaagattaccactgatgctcagcctgcaatattggaagtcataccagagactaggcctcctaaagtaatcactaggcatcagtacagtaaccctgcatttgaggtgtccattacttctaactctggtgcaggggagtcttctgcttctgaccatgtgctggtggaaggtttttctggtggccactcaattggtgaacacattccattgcaagaccttgcacccagcaggccttcattctctgagaccatagaagatgaaactgcttttagcagcagtactcctaaacaaggctctagatctgaaaggcctaaaagttactataataggcgaagatatcagcaagtacaagttactgaccctgtgtttatttcaagaccacggtcacttgtcacgtttgataacccagcctttgatgaatctgttgacctgatatttgaaagagatgttgcagaaataactgcagcacctcatgcagactttacagatatcacaaagctcacaaagcctgcatatcacagaggcccatctggccatgtccgtgtcagtaggcttggacatagagctaatataaaaactagaagtggtcttacaatagggccacaaagccatttttactacgatgtcagcagcattgaccctgcagaatcttttgagctgcaggcacttggcaatgtatccagtgctgaacaaacaggagaagcagtaatctcctctggcacaggagactttgaaattataagccttgaagacagtattttggaatcctacaatgatgaggatttaatagacgtgtttgaggatgtagctagagatttgcatttattagttggagaaagaaggcagcaaccgatccaagttcaacgttacataaagcctttttcttttgttaatgagggagtacacataattcacccaggatctgagtcagatttttggctgcctcctgtaacgcctgacagcacacctgcaatagtgattgacattttggactcctctgcagattactatctgcatccaagtttaataaaaaaacgcaaacgcaaacatttttttttttaatttgcagatggcggtttggcttcctgcacagaataaattttaccttccaccacagcccagcaccaaggtcttaagcacggatgaatatgtctccagaacaaatattttttatcatgctagcagtgaacgtcttcttactgtggggcaccctttttatgaaatttataaagaagaacgttctgaagaggttatagttcctaaagtatctcctaatcagtaccgggtattccgcttgctacttccagaccctaacaattttgcatttggagataagtcattatttgatcctgaaaaagaaagacttgtttggggcttaagaggattagaaataggtagggggcaaccattaggtataagtgttacgggtcatccaacatttgacagatacaatgatgtagaaaacccaaacaaaaatcttgctggacatggaggtggaacagacagcagggttaacatgggtttagaccctaaacaaactcagatgtttatgatagggtgcaaaccagctttaggtgaacactggtctttaactagatggtgcacaggacaggtacacactgcaggacaatgtccaccaatagaactgagaaacacaacaatagaagatggagatatggtagatatagggtttggtgcaatggattttaaggctttgcagcattataagtcaggagttccaattgacatagtaaattctgcatgcaaatatccagactacctcaaaatggcaaatgagccttatggagatagatgttttttttttgtaagaagagagcaactgtatgccagacatattatgtccagatctggcacacaaggtttagaaccagtccccaaagatacctatgcaacaagagaagacaataacataggaacaactaattacttctccacacctagtggctctctggtttctagtgagggacaactgtttaacaggccttactggatccagcgctcgcagggcaagaataatgggattgcatggggcaatcagctgtttttaacagtagtggacaacacacgaggaactcccttaactataaacatagggcaacaagacaagccagaagaaggaaattatgttccttcatcatacagaacctacctcagacatgttgaagaatatgaagtaagcataattgtgcagctgtgcaaagttaagctgtcccctgaaaatctagcaataattcatactatggatcctaatattattgaggattggcacctaaatgtcactcctccatctggtactttagatgacacatataggtacataaactctcttgctactaagtgccctactaatatacctccaaaaactaacgttgatccttttgcagactttaaattttgggaagtagatcttaaagataaaatgactgaacagttagaccaaactccactgggtcgcaaatttttattccagacaaatgtgttacgtcctagatctgtaaaagtacgttctacctcgcacgtttctgtcaaacgaaaagctgtgaaacgcaaacgcaaataatgtgtcattgattacttgtgaataaacagataattatttatgtccagttgttgtggtcattgtttactgactgaccggcaccgcaccctgcacatattgcacacagcaccagcaaaggcaggctaactcagacaagccggcacctgaattaagcttttaatctttttaatcttaaaaatccctttaatcttttggagcgaccgttattggtttggagtgacgcccggacattcctgacaagaccggattcgttcgaccg

SEQ ID NO: 14 - DNA, 7348 bp, Human papillomavirus type 63
gttaacaactatcaggcgattctctagttctaacacgaacgtttacggtcgttgccagctttttccttataaaactctggtgggaatttctcttgggacagatggacctgacatctgtacattcggttcgggatctgagttctgctctccgtatcccatttattgatttggttgttccttgcaatttttgcttgaaatttcttacaaatgctgaaaaattgctgtttgattattttgacttgcatcttatctggcgagataatttcgtgtttgcttgttgtcagtgctgtgctaggcatgttagtctgcttgagtttatgctttattatcaggagtctttcgaggtatctgaagtagaagaattacttaatcaacctcttgtaaatattggtttaaggtgtgttacatgcacaaaaaaactgactgtttcagaaaagttagctgttgtttctgctggagaaagagttcataaagtaaggaacaaattcaaagcaaagtgcagtttgtgcagactctacattatatagtttgtgcagactctatataattaacaatggtgggagagcagccaaatataggtgatttggtgagtcaagaagaaccaagcgtcctagatctaaattgttatgaggatatacctgctgaggaggaggagtctgaatatccatatgcaattgtgcttccttgtggtttgtgcgatcagctgttaaggctgacctgcgtttctgacctgtctactcttacgcgtctggaggagctgctgttaggctcactgaggatcgtgtgtcccctgtgtgccattcgacaccaacgacactaagatgaccgacagaggtacaaataatgatgattggtatattgtggatgaggcagaatgtcgggatgatgatgagagcgaattggaggatttggaggacacctataattcattgtttaatagatctgaaagtgacatatcagatctattagacgatacgcagcaaagtcagggaaattccctggaactgttccacttacaggagcacttgcagaacgagcaggacctaaataccctaaaacgaaagtacttaaacagtcctccgcaggcaagtgccacagagactgcctgcaatagcctcagtcccagattggaatctataacaatttcgcagagggaaaaaaaggcaagaaagcaactatttacacaaaatgacagtggcatagagttatcgctatgccaggatgaagttgacaatattaacgaagcgcttcaggagcaggtagacatcgtacagtctctgggaggtggggtgcgtgactgtataggagtggacattttgaaatgcagtaatacaagatctgctctacttgccaaatttaaagacacagtaggtgtcagttttactgacctcaccagagcatacaaaaacaacaagacatgctgtagttactgggtcatagcagtgtggggagtaacatctacgtctgtggacgttgtgaaaactgtattccaagttcagtgtaattatatgcatgtagaacattgtttaactgaaaaaaataagtttctaattgtattagctggctttaaagctcaaaaaagtagagaaacagtgttaaatctcgtaactagcagtttgaatgtgcaaagtaattacataatggctgaaccaccaaaaaatagaagtatggcggcagcgttatattggtataggagatctatgtctccagctgtatatacctggggagaaatgccagattggatggcgcagcagacattgttgaatcatcaattagcatcagaaaagcattttgaattgtcacaaatggtacaatgggcttatgataatggctatacagatgaaagtgatattgcatactattatgctattttagcagaagaagatgaaaatgcaaaagcattcttggcttctaatgcacaagcaaaatatgttaaggactgtgctagaatggttagtcattacaaaagggcagaaatgagtagtatgtctatgtcagcatggatttataaaagactggaggaagttgaaaatggtggtgactggaaacatattgtaaagttcttgaggtttcaagaagtagaatttataagtttcatgatagcatttaaggaattgttaagtggtaaaccaaagaaaaattgtcttgtaatatatggtccaccaaatactggtaaatctatgttttgtatgagtttgttgagagtattaaaaggaaaagtaatatcttatgtaaatagcaaaagtcaattttggttgcaaccactagctagcactaaaatagcattattagatgatgcaacaaaaccagcatgggattatattgatttatttttgagaaatgctttagatgggaatcctatttgtgtagatctgaaacataaggcaccacaacaaataaaatgtcctccacttatgataacttctaatataaatgttaaggctgatgtatgttggatgtatttacatagtaggataacatgttttgaatttaaacaaccttttccatttgatgaaaatggtcaaccggcattttccttaacagacatcaattggaaatctttttttgaaaggttttggagccagttagacttaagtgaccaagaagacgaggagagtgatggaaagcctcaacaaccgcttagactggctacaagagcagcttctaactctatatgagaaagacagtaaagatattgaagatcagataatgcagtggaatctacttagacaggaacaagtgttattccactatgcccgaaaaaagggaataatgcgacttggcctgcaagttgtgccttcccttgcagcttcccaggataaagcaaaaacagctatagaaatgactctttatcttagtggcctcagagactcacaatatggttctgaacagtggtctttacaagatactagcagagaaatctttttagcaccaccagatcatacattcaaaaagggagggcaaacaattgaggtaatctatgatgaggatcccaataatagcaccagacatactgtatggcgccatatatattatcaaaacggtgataacagatggagaaaagcagctagtgatgtagatgttcatggtgtgttttatttagaatatgatggtgtcaaaaactactatgttgactttcaagaagaggccaatcgatatagcaaaacaggtcgatatactgttcaatatgagggtaaaaggttcacaaatgttatgtctcctgtcaatagctccccactacggacttctgggtctcctacagacaccaacccagccacccaaggacaatccacccaaactgccagaaaagcagagacgaaggggtcgagacaccacccgaaatcgccggctgttcgcaagcgacggccctacggacgaagaaggtccagaagtcccagagataccaccctcagacgaggagaaggagaatcggccagagcctctgccggtagtggagaacgggtggcattcatttctccgggagacgttggaacatcaactaggtcgcctccaaagggaggtcaatcaagacttcgaagacttatacaggaggctcgggatccacccataatttgtctgaaggggggccctaatcaacttaagtgcttaaggtataggattaaagcttcaaattcatctgactttgaaagtatcagtactacatggcattgggtacataataaatgcacagatagagtaggtcatgcacgtatgctggtgcgttttatatcaacagaacaacgtgaccgatttttagataaggtggtggtgcctaaatctgtttctgttattttaggggcatttgacggttcctaagggtgggtgttggggtatattttgtaatcatgttaagagtacgtaaacgacgagctgctccacaagatatttatcctgcttgtaaggttgcaaacaattgcccccctgatatacaaaataaaattgaacaaacaacagttgctgacaagattttacaatatgggagtttgggaatattcctgggaggtttgggtattggtactggcaagggtgggggtggccggtatggttatacacctctaggggacagtggtgcggtgcgagttggtggcagaagtacacctgtaagaccaacagtacctgtggagactgtaggaccaagggatatattacctatagattcattggatcctttagggccctcagtcattgaactagaagatattccagccacaacagtggaagtagtggctgaagtgcatcccatatctgatactccacaaataccggcacctactactgatgaatctagttcagctgttcttcatattccacaagaaagtcctgctgcacgtacaatcacacgttcccaatacaataatcctttattcaggatcacagctagtgcagacatagcatcaggtgaagcttcagcatctgataatatttttatagatgtagatacgccgggtcaaatagtaggacaagaaataccactagttaattttgatatgggacctatatctactgaaggtgagcttgaaactgagttcacaactagtacaccaagaaccacacaagtacaggaaaggcctacacgtttctataatagacgctattatgaacaagtgccagttactgcacctgaatttatcacaaggcctgcttccttagttacttttgagaatcctgcatttgaaaggagtgtttctttgatttttgaacaagatttagaagatattttaaatgctcctgatcaggattttagagacattgtttatttaagcagaccaacatacagtcgtgcccctgatggccgcatgcgcctaagccgcctgggacgcagagccactataagtaccagaagtggtgttactataggtgctcaatcacacttttatatggatattagctctatctcctcaaatgatggcattgagttacaaacactgggtgaagcttctggcgagactgtggtgcaaagttctcttgctgcatcggatcctattgaagcagaacattcattcattgaaccagcaccatctatagatagttatgatattgtttcacttcagtctgagacttattcagatgaacatttgttagatatgtatgaacctgtaggttcttccttgcaattacaaatatcagacgtcagaggtcggccaactgttattgatattccctttagaccccgcaggcctccattaggtcctataaatgctggtgttgatatctatagtccaactgctagtgttggatcacctactataaatcctactgatcttgacattccattaattattatacatttagataattcaacaggggattatgatttacatccaagtttgcgtaaacgtcgcaaattagttcatatttgatattttacagatggctgtttggcttcctgcccagaataagttttaccttcctacccaaccgatcaccaagattctaagcagcgatgattatgtgtctcgcaccaacatcttctatcacgctaccagtgatcgactgctcattgtgggacacccgctctatgaggttacccgtgcaaatgataacactatgactgtgcctaaagtttctccaaatcagtatagagtctttcgtgttagatttccagatcctaaccgatttgcctttggagataaggatatttttgacccagaaactgagagactagtttggggtcttagaggcatagaaatcggtaggggtcaaccattaggtgtgggtatttcaggcaatccattattaaataggtttgatgatgctgaaaatcctagcagatataataatacacatgcaactggtgataataggcaaaatgttgcttttgatgcaaaacaaacccaaatgtttctaattggctgtacaccagccactggggaacactggtcaatagctcgacgctgtgcaggaacacagtttcagcttggagattgtcctcctatagaattagttaacacagttattgaggatggtgatatgtttgacataggtctaggtgctatggactttggttctttgcaagcaaacaaagcagatgctcctttggatattgcaggcactgtctgcaaatatccagattatattaaaatgggacaggaagtacatggtaattctctgtttttctttgctcgcagagaacaaatgtatttaaggcatgtatttacacatgctggaattgttagtgaaaaagagaaagtccctaccagtgcatatattgctgctaaagccgagcaaccccaaaatactattgctacagataattattttgtagctcccagtggatctttagtgtcctctgatgtgcaaatttttaataggccctattggttacaacgttctcaaggacagaacaatggtatctgttggagaaatgagttatttgtaactgtagctgataataccagaggaaccacgatgaatataaatgttcttaacaaagcaacccctgagacttatgatagcgcagattataatgagtatactcgccatgtggaggaatatgagttatcctttatagttcagctttgtaaggtaaaactaacacctgaaaatttagcatttttgcataatatggatccaacaattatcgattcctggcagttaacagtttctcaacctcctgcaaatgctatagaggacaagtatagatttattgaatcattagcaacaaaatgtcctgataacgtgcccccacccactcctactgatccttacaaagatttacgtttttgggatgtagacctcagtgagcgaatgtcggagcagcttgatcaatttcctttaggccgcaaatttttgtatcaaagtggtcttgcacagcgttctgttccaaaaactgtgaatttcagaaaacgtagatcctccaatactactgtggccaaacggaggcgacgggcctgaatatacatgtgaatgttgaattatataatgtgaattgtgaattcttgactttggcacttgcactttattcttggcatactgatacttgaaacttgttcaatgcttgaaggttacacacctgtacagtattgttaataaacgtttatgctgctgtcatttacctgtcttcgagtcattattgcctagtcatatagcctcatgacttggcatgcaattggtatgtggcagatacttcaaacaggatactggtatcctttttggcgcgcgcgcgaattttgaagttaccactgttccaacttgttctgagacgtctggatctgatcccgaccgctgtcgttactgccaaagacgaaagtggtaggcgcgaaccgtttgtggtttccctggggctagcagaaactctttaggttgcgaccgttttcggtcgggccaataatctctttcgatcgttgtt

SEQ ID NO: 15 - DNA, 7614 bp, Human papillomavirus type 41
acaatcataatcatcgccctttcgtgttatttcttgtaacgaattcgttacaaaacacacacacagtatataagatagaggaacggattggtacaccacagatggcatcaacaagcggtgtgggatccgtcgggcctgcaagctgttgcgagacgcagaagccacataccatacgggagttgtgtttggcgcagcagataacttatccatgcatacagctctgctgccattattgctataagatccttagcgtattggatatttacgctttcgaccagagctgtctgtacttatcctggggagaaggggggccaacgggtatttgttctcagtgtactagagtgcttgcaaggctggagttcactgcacggcacgaagtgtcttgtgcagccagccgtctgccgcactttataggacagagcctcagcgaccttgaggtgaggtgtgtgaggtgcctagctcttctacaatctgtggaaaaggattacatattgcgggaagacttgtctgtgcatagaattggcgggatctggaggggaacttgtgttcgatgtatggtaggactgtattagctgtgagactaatatactgtttgctgtattgtattgctgtaatcgtgcgtaaattgctataccctgtaataatgagagggaatagtgttgacctgcaagaaattgtgcttgttcagcagggggaggtacctgagaatgctgcagtgcattcaggggagcattctgatgatgagggtgagagcgaggaggaggagcgggaacaggtgcagcaagtccccacacccaggagaacattatacctggtagagagtcagtgtccattttgccaggctatcatacgatttgtatgcgtagcaagcaacactgggatacggaatctacaggcactcctggtcaacagtcaccttgacctcgcttgtcacgcctgtgtcgagcagaatggcgtccagggtctcagacaccggcaatggcaatgaaaacaaagagaatgaaggtacagtggcatctgatcattctgaggcgcgttgtagctatatattatttgaggctgaatgtagcgatggcggggacgatgaggaaagtatggaggatagcttggtggaagaccttgtggatgatgcttctgtgcatcagggaaattccttgtcgctgtttcatgcccaaactgtcgaggaatacgagggagagatccagagcctaaaacgaaagtttatcctgagtcccttgcatagggatgtggcagaactaagcccgcgtctggcgggtgtttccctggaagaaaaccgtgggaaaaaggctcgcaaatctctgttccacgatgacagtggcatagacagcagcgcagtggaagtctcccagctatctagtacgccatcagctccagggccagacatccggctgcctaaaccctcagatatagatctagagccactgttccaaagccgccagcgctgtacgcatatgtatagcaaatttaaagctgtgtacggggttagctttacagatataaccaggccattcaaaagcgacaaaacaacatcacagcattgggttgtggccgcctactatttagcttttgatagtgagataagtgctatggaggttttgctgcgacaacaatgccaatttttatacattgacaacaatgatggcattatactgttcttcctggaatacaacgtgcagaaatctaggactacagtgtacaattggttcacagccaatttccattataatgaaaatagaatgctagctaatccgccaaggacacgaaacatgcctgctgctttattcttctatcatagatttatgggtacagggggtataaaacatggcgcaatgccagaaataattgtaaaccagtgcgtggtgtctaatcagcagacagacacctttgaattatcacgtatggtacagtgggcactggacaacgatctgcaagatgaacatatgttagctttagagtatgctttgcttgctgaaagtgatggcaatgcgcgggcttttttaaagcagaataatcagccaatgatagtgaagaattgtagcataatggttagacactacaagacagcgctggtcgcaaaaatgtctatttcacagtatgtgaataagcggtgtctggaccatggggaagctgatgaaaacagctggcggggaattgtgcattttctgaggtatcaaggtcaggaattcctgcccttcatgtgtaaaatgcacaatttcctacaccatagaccaaagaaatcaacacttgtattatgtggaccgtcggacacaggcaaatcatattttgccaatggtcttaacaaatttttggatggacacgtgctgagctttgtcagcaatgggtcacatttttggttatcaccattacgtggggcacggtgctgtctaatagacgatgcgaccctcacgttttggaggtacgcggaccaaaacatgagggcactgctagatggatatgagatttccattgatgcaaaacacagaaacccaatgcaaactagagcaccaccattaataataaccacaaatgaggacattatgcgattagatgaattcaaatatctgcaaaccagaacaatgtatgtgtactttaacaagccatttcctcttaaaggaaatgggcaaccgttatattacattgatggttatacatggaactctttttttaggaaattttggcgtcacctaaatctaaaagaccctgaggatgagtcagatggagagactcctggaacgattagactatatacaagagcagatactgacactatatgagaaagatagtgttgacctagaggatcatataaggctatggaatctgctaaggagggaaaatgcaatctggtatgtactcagacaggaaggacacgcaagggtcggcggcagagcggtgccggcaatgacggtatcggaagccaatgccaaattcgcaatagaaatgcagataaagctagaatcactaaaggccagtccctatgcggccgagggctggtcattgcaagaaaccaccaaggaacggtacttggctgaaccgtctcggacatttaagaaattagggcagccagttaccctaatgtttgacaatgatcccgaaaaccttacagaagttgtattgtggaaatgggtttattatattacaccaacagatgaatggtataaagctagaggtggcattgatgacactggtatatactacattgaccacgagtctgttaaaatgtactatgtgagatttgacatggaagcggagaactttagcgagacaggcactgtcacctaccggctaggcagcgccctggtaaatgtacctgaacctgtaactgttaccgacagctcctccacgagggagagaaccccaaaggtactacgaccgcaggggtcgagacgacgcagaaacgaggaaacgggggagccggtcgccccagcccctaagcgaagacgaggagcttacggacgcagatcctccccgaaggcccaacgcaggaccgcggcgtcgcctgtttctagaggaaacggaggatcgtctgacttcacttctggagagtctgacgaaggacatcgagtcagacatagagcacttcgaaagaaaactgcgggtgttgctccagcagaaggacactatctagttggcgccaaaggtccagtgaatagcctgcggtgcttaaggtacaaatggaaaaacaagtatagcggtgacataatgtatctggggactactttcacatggacggagtctgacgggacagaacggtgtgggtcggggcgctttttttgtgctttctctaatgaaacaaaaagagaaaagttcctcaaatctgtcaagattcctaaaaacattgggctgtttcgcgcacacgcagaaaagctgtgacctgtgtatcattaaacaatgcttgctaggcaaagggttaaacgcgctaatcctgaacaactgtataagacatgcaaagcaacggggggcgattgtccacccgatgttattaaacgctatgagcaaactacacctgctgatagtatattaaagtatgggagtgtaggggttttctttggcggtctgggcattggcacaggacgtggtggcggtggcacagtgcttggggctggggcagttgggggacgcccgtccatatccagtggtgcaattggtccccgggatattttgccaattgaatcaggggggccttcactggcagaggaaatacctctgcttcccatggcaccccgtgtgccaaggcctacagatccctttcggccgtcagtgctggaagagccttttattataaggcctcctgaacgcccaaacattttgcatgagcagcgtttccctacagacgctgcaccatttgacaatggcaacacagaaatcacaaccattcctagccaatatgatgttagtgggggaggggttgacattcagataattgaactccctagtgtgaatgaccccggtccctcggttgttacccgcacacaatacaacaatccaacgtttgaggtggaggtgtccactgacattagtggagaaacctcatcaacggacaacattattgtaggagctgaaagcggtggcacatccgtaggtgacaatgctgaactgatacctttgctagatatatcccggggggacacaattgacacaacaatacttgcccctggcgaggaggagactgcctttgtgaccagcactcctgaacgtgtgcctatacaggagcgattacctattaggccctatggcagacagtatcagcaagtgcgagttaccgaccctgaatttttagacagcgctgcagtacttgtctctttagagaatccagtgtttgatgcagacattactctcacgtttgaggatgatctgcagcaggcactacgtagtgacacagacctgcgggacgtgcgtcgcctcagtagaccttattaccagaggcgcactactggccttcgtgttagtcgcctggggcaacgtcggggtactatatccacgcgctctggtgttcaggtaggctccgctgctcattttttccaggacattagtccaatcggccaggctattgagccaattgatgcaattgaactagatgtactgggtgagcaatccggtgaggggactattgtgagaggagaccctacgccttctattgagcaagacataggactaaccgctttgggggacaacattgaaaatgaattgcaggaaatagatttattaactgcggatggtgaagaagaccaggagggcagagacctgcagttggtattttccactggcaatgatgaggtggttgatattatgactatacctatacgtgcaggcggggatgacaggccttcagtatttatttttagcgatgatggcactcacattgtctatcctactagcacaacagccaccaccccactcgtgcctgcacagcccagcgatgtgccctacattgttgttgacttgtatagtggaagtatggattatgatatacatcctagcctgttgcgcaggaaacgtaaaaaacgcaaacgtgtttatttttcagatggccgtgtggcttccaggcccaaatagattttacttaccccctcaacctatacaacggacattgaacacagaggaatacgtgagacgcaccagtactttcctccatgctgccactgaccgtttgcttactgttggacatccattttacaatattactaatgcggatggcaaagaggtggtccctaaagtttcctctaatcagttcagggccttccgtgtccgtttcccaaatcccaatacctttgcattttgtgataagtccctttttaaccctgacaaggagcgtctggtctggggtattcgtgggattgaggtttctaggggacagcccttaggtattggtgtaacagggaaccctttttttaataagtttgatgatgctgaaaatccctacaatggtataaacaaaaataacattactgaccaaggttcagactcaaggttgagcattgcatttgaccctaagcaaacacagctgctgatagtaggtgctaaacctgcaaagggtgagtactgggacgttgctgcaacatgtgaaaaccctccactgaccaaagcagatgacaaatgtcctgctctagagcttaagtcctcatacattgaggatgcagacatgagtgacataggcctgggaaacttgaatttttctacactgcagagaaacaaatccgatgccccattagatattgtggattctatctgcaaatatcctgactacctgcaaatgatagaagaactatatggagaccacatgtttttctatgtgcggcgtgaagctctgtatgctaggcatataatgcaacacgcgggcaagatggatgctgagcaatttcccacttctctgtacatagactcctctgtagaaggtgagaaattaaattccttgcagcgcactgataggtatttcatgacacccagcggctccctggtagctactgagcagcagctgtttaacaggcccttttggctgcagagatcccagggccataacaatggcatactgtggcacaacgaggcctttgtaacattggttgacactaccaggggaactaactttaccatcagtgttcctgagggggatgcttcttcatataacaattctaagttttttgagtttttaaggcacaccgaggagtttcagcttgcctttattctacagctgtgtaaggtagaccttacccctgagaatttggcttacatacacacaatggatccatccattattgaagactggcatttagctgtcacttcacctcccaattctgtactggaggatcattataggtacatactgtccattgcaactaaatgtccctctaaggatgcagatgatacctccactgacccatacaaagatcttaagttttgggaggttgatctacgggatcgtatgacagagcaattggaccagactccccttggcaggaagtttttgtttcaaactggtatcactcagtcatcatcaaataagcgggtgtccacgcagtctactgcccttactacctacaggcggcctactaagcgccgccggaaggcttaaacgaattgctggtattgtggtgcggtgtcctcgacggtccatgtgtcatcttataatcacttggtcagtccagggtacaccactccattatctatttacttcgcatgtatttctctgttatgttcctgtatgggttatgaatgtgttaataaaatatgttggtaacgctgtgcacgggtttgttcacgttcatgtctcatgatttggcacccctgtattcccgccgccgcccgggggatcgcagatataatccccaaacccaaagcgttccaacattggcaaacgtctctggccccgatacaactgaaacggtctgtcttgccaatagccccatctggcggggattcaactgaaacggtgtgtactgccaagtaacatttttgttattggaacgcctccggtgctggcggaagcgcaaggatttaggcgcgaagacagttttattgccaaaaccttttggttgctgccaatagcaggcgtggtctcaacgaattcgttgcggcaataggtatgtaccatggttatga

SEQ ID NO: 16 - DNA, 7879 bp, Phocoena spinipinnis papillomavirus
gttaacaattataagagaaaaagaaggttaccagaggataccgaaggcggtgtcagctatatgaagcattaaaatatccaggaagaacctcaaaccttaaggtaagcatggcggaggaaagcccatgcacaattaaatctttgtgccttgcatttggactgcgttttgatgaattgcttatatcttgtgtattttgtagaacaaatctaaggacctttgaagtatggtcgtttatgaccaggaatttaaaggtcttatggaggaaaggatttccttttgcctgctgtcctaaatgcctggaggtacaggcactggtggcttggctaaggcattttgaaagatctggaaatgcaaaggcggtagaagaggacaccggcgaatccctgggggacctgcccatgagatgtgtaggatgctttaagcctatgtcagcctctgaaaagcagttccagatagaggataaaaggccatttaccaaggtttctggatattggagagggttttgcctcaactgcctgacaacccccccaccgctgaccaggtattttatatcggtcactaatactggacgtacgccgcttataagctggggatttgatcccccaccacgccagctgtccgaaagcggctcttctgctagcagctggactatcacaacaacaacaacaggttcctcgtccaatgctgatgagcacccattgagcgacgcggaaagcgatggagaaaccgaggcattaatctagcaacacaggaaaaaaaaaaaagaggcgcctacgcactttgtatataagactgattgtaaatactaggtggagtaactaggaaaaactctaacaaaaaggtgttgtgctgtttattaaagtgtacggagccggttcaataaggacaacaacatggacaacacaccaggtacagacccattggaaggggggagcagtgactgggtacttttagaggccccagacgatggggagggggactctgaagaggaatatgatgaggaatttgataggggggaagatctagtagattttatagatgatagtgtaaatgtacaggacgttagcgactcagatttctatagaaggctacaagtagagcaacagagggaggatgatcagagggcggcgcatgtactaaaacggaaatttttagatagcccgaaaacgaaagcagacagtgatctaagcccacgtttagaggcaatatcactgcaagagagatcaggacgggcaaggagaaaactatacaaaaacagcactgtggacgatagtggacatggggattccctggaagcgtcgtgtttggagtcgttggcaggacgaggcgaacaggtacccatatcccaaagcgcagaaccgtgggagggggctacaccgacagtggtgtgtacagcaaaaacagcacaggaagtgcagagtactgcacagcaagaggattatacaagtcaggtcacgcagctaatgcaggcagggaagccaaggaatgttttgctagcattatgcaaggatgcatatggctgctcgttttcagatctcacgagatcatataagagtgataaaacagtctgtggggactgggtatgcttgatagcgggggttccttgttctctagaagaggctattacagatttgctaaagcctcacagcgattatacacatgtaaacatatctacctgtaggtatggcctattattattgttgttagtaaggtggaagacggccaaatgcagggaaacagtgcagaaacttctaggggggctcatgtctgttgaaaagcatcagatggtcctagaacccccaaaaataagacatccagcaacagccatgttctggtataaaaggacattggcaaacgcttcagtggtaacaggagagacaccagaatggatactaaagcaagtaagtttgcaggaacaaataggagccgcagccacattttcattgtcggcaatggtgcagtgggcatatgataatggtctggagggggagagtgagattgcatatggatatgcacagctagcagaagaggatactaatgcagaagcctttctccgtagcaatgcacaagcaaaacatgtaaaggactgtgcaattatggtgaggcattacaggcgtgcagaaatgtgcaaaatgaacatagcacagtggattaagctcaggtgttccaaggtggagggagagggagattggagacccatcatgaagtttctaaagtttcaaaaagtagagatattggcatttctaacatttatgagacatttcctaagaggaacccctaagagaaactgtatggtgctcttgggacccccaaatacaggcaaatcattatttggaatgagcctaatgcactttctaggcggtaaaatcatttcacatgttaactcaggaagccatttttggttgcagcctttgttagagtgtaaggtagccatgctggatgatgcaaccacaagcacgtgggactacatggatatttacctaagaaacatgttagatggtaacactgtatgcctggatgcaaaacacaaggcccctatgcagcttaagtgcccaccattaattgtaactacaaatgttgatgtaacagcaaatgataagtggaaatacctgcatagcagactaaaggtgtttacctttccaaatctatgtccattaaattgtaggggtgatccagaattccagttaaccccagaaaactggaaggcatttctcgaaaagtgctggactagtttaggattggaagacctgctgaaagacggggatggagaacctttgcagccgcttagatgtgctgcaagagcagcagatggaactgattgataaggacagtggctgccttaaggacatcataagctattatgccttattgaggagagaggctgtgcttctctttgctgcaaatgtacgtgacattaaaaaggtggggctcacagttgtcccaccaaagcaggtgtgtgaagcaaatgccaagcaggccatagaaatgcatcttgtattatgcagcttatctgagagcacatatggacaggaaccctggtatctggcacaggtctcacatgacatgtatatgttacgtcccactggcacatttaaaaaaaatggtaagagggttcttgtaacatttgatggggatgagagcaatctgatggaatatatgtgttgggaagcagtgtacaaacaacgtcaaaatggacaatggtcttgtgttaaatccattgtatctcatgaggggatctattatgactgtgaagggtacagagatatgtatgtggactttgcccgtgaagcagcaaagtatggaaacggtggagagtggtctgtacaatgtgatggacagggcataactgattgtgcacttgtatctagcaccagcaccccttccactttggacacctccctggacgcatccctgggaaacaccttactatcgccgggtcctggacggaataaacaaaccccaaaggccaaggggcgacgtggaaggaagagaaagcttgacccagcagagcccgacggcgtacgcttcggtccccccccatcgcccccaccatcaccaaagccagcaccagtcccaccaccatcaccaccatcaccacctccaacgccccctgagcccccaggcggcagcggaggaagcctcaactccacagggaccaccgaccccggcagttgcacaggaaacagtgatacctgtggaggacccagtgacagtgactgtgacaattggggttccaaacggcccggaacttgtcctgacattccaactctcctaatctcaggtgggcctaaccaggtaaaatgtctgcgttatagattgaggcggcatcaccgcaaggcatataggtcatgctccacaacatggtcttggataggggatgatctacaggaccacactgaacacaggatctgcctctccttctacagtgaagcacaacgtgtgaactttcaaaagactgttagactgcccaaaggtgttcgtgttggcagcgtaaatctgcctttctagctctgttctcttttgtgtgcatttgtgcatatctttgtacatacatctctttgtacatagccctgtaaaatctttttttatatattgttgatgtgtgtgtaagcagatggtccgtgtaaaacgcaggcggcgtgcagccgagggagacctttatgcaggctgcaggcgtgggcaggattgtccagacgatattaaacctaaatttgagcaagatacatgggcggatagatttcttaagtggtttagcagcatcatctacttgggtaaccttggaataagtactggtcgcggtgctggtggatctacagggtacgttccagttggctcaggtggtggacgtggagttaggcctgcaatgggaggccagccttcacgcccaaatgttgttgttgagaatgttggccctgcagaggttcctgttgatggagctgtggacgcttctgcaccatctgtcatcaccccttcagagtccacagttgtggtgggtgggtctacaacaccacatgaggagattcccttggtccccctgcatcctgaggttggcccagaccctgaacctggtttgccccttcccccaccggagtccggaggccctgcagtcctggatgtgactttgaatgtgacctctacatacacacatgacccctcaatcatacacccacgtgtctccagcttgggagagagcgcaggtgcagaggcgcctctatttcccactatctcactccagcctttggatgtgtctcttctgcctggtgagagttcttttcagcctcatgcatacattgatctttctggttcctttgaagaaattgagctggatgcatttactagtggtcctcaggatcctcaaacatcaacacctatgtccagagttgacagtggcttacggtctgtgagacgtgcatatagtaggcgtacaggtgctttacgaaggttgtaccaccgcctcacgcagcaggtgcgggtaaacaggccagagtttttaaggcggccttcacagcttgtttcatatgtctttgataatgcagcctttgaccctgatacaacattgcattttcctcaagcatctgaggatgttttgcaggcccctgaccttgattttcaggatgtagggacactccacaggcctatatattctactgagggtggatatgtccgtgtaagccgctttggggaacgtgaaaccattaggacccgctctggtgccgctataggcgccagggtacacttttacacagaccttagcagcatacaaagctttgcagagcaattaccttctgttggatccctaggccctgatgttgcagaccctggaatagagttacatctctttggggaaggcacaggagacacatctatagcggatgcccagggaggtggggtctcattaagcaatggaacccttcatactgaaacagagttcacaaatgcttctaatggttctttacactcagaatactctaacagcatgctcctggattcatacacagaaacatttaatgatgcacaactggcattgatggattcagaggggtccacacaggttttgtccattccagagcttgctcgcccggtgaggggatttgctgaatccacagggggcctttctgtctcataccctgtagatatggagatatctgggtcctctacatcgtttatccacaatattccaggacctccatcaatactgcttttttaccctgatagttcaccctcattttaccttcatccaagccttctacgtcgcaaacgaaaacgtgttttttattaattgtttttcagatggcttctacctcctactggctcccgtccacggacaagctgttcctgccccctcctgcgcctgtgtcaaaaatattaagcacagatgcttttgtcacacgtttagatatattttaccatgctggaacaggccgccagcttcttgtagggcatccatactttgatgtgctaggtgaaaatgataagttaattgctaagaaggtttctggtaatcagtaccgtgctgcacgttttacactacctgatcctaacagatttgctttgcaggaccccactatatatgaccctgaccgtgagcgcttggtatgggcatgccgtgggcttcaggtcggaaggggcttgcctcttggagggggaacaacaggccacccatattacaataaggccaaagatactgaaaatcctaatagtggcaagtatcctaaaacaggtgaaggggacaataggcagaatgtgtcctttgaccctaagcaggttcaaatggtgtttgtgggctgctcaccctgtgttggagaacattgggacaaggttacaagcacctgtgcagatcaggtacataaagaaggtgactgccctgcaattgaattggtatccagtcatatacaggatggggacatgtgtgatattggctttggggcaatcaacaacaaaactctacaggaatcacgttcagaggtgcctttggacattgtgtcctctatttgcaaacatccagacatacttcagatgtctaatgacccctttgggaactctatgtggttctttgccaaaagggagcagatgtatgtaagacatatgtgggccagacgtggaactgtttcagaaaaggttccagaccccgccaatggtggtgcacatgctcatgaattttacctgtcccctaagaatgcagaggaaaaggcaatggcctctaccatatactctgcaacaccaagtggctcacttattacaagtgatggccagctttttaacaggccttattggatacaaacagcccagggtaaaaataatggtatatgttgggggaatgaggtgtttgttactgtggctgataataccagaagcaccaatataaccatttctgtgaaggacccaagcaaaaataatgcccatcagggtgcatatgaggctgaccattttaaaatatacacaaggcatatggaggaatatgagtttagtttcatctttcagctctgcaaggtgcctttaaccccagaagtgttggcccaacttaacaacatgaactctaaaatcattgaaaagtggaatgttggctttgcaaccgctgcgcctgcatcctcctctcttgctgagcattacagatatattaattcactggctactaaatgcccacctgccccagaggacactgaagaaaaggacccttatgaaggtgaatcatattggaatattgatctttctgaggccttttcttcagagcttgattcctttcctttaggaagaaagtttttataccaggcaagtaaatctattcgtgcccccagtcgctcgaccaccaaacgccccgctgccaaatcccctgtaaagcactccagtaaacgtgcacgcagataacctgaatcataattgctaatcatgatgtgtatgtttgtgacatatatgtttgggacgtgtattgcatgatactttgcatgtgcatgtatgtgtgtttaccacaatcctgattgctaatacataatttgcatgtttttgtgtttgcatgtgcctatgtgtgtgttgaatgattaataaacagggtatcatttccgctcgcgccctataattgtcgcccgtgttgtctgtccactgcttgttgtccgtcagtattgcttctgcttctcccgcgcgcgggacattcatatttttgaatgtaaacggtttggtgttgctttcccgcctaaccagtacctttaaagtttcctggcgattctccattcaaaaaattggtgagtaatgaatttttggcgtcttagggattagttgaacaaaaggtaagtatgcctgttgtagcctgtttcaaggtaagtaggcacataaatacctttaccggtttggtcatgctgctcttttatatatcatgatgatgatt

SEQ ID NO: 17 - DNA, 7304 bp, Psittacus erithacus timneh papillomavirus
atgcgaacaatgcgctaccactaccccacggagacggacgacagcagtcctgatgaaggtgagactggcaaccatgctgtaacccacttgctaatgcagctgcaggaacaactgcatgcacttaactatcccacagatgatagcgacgactccacagacggtgaggaattagtattccgattaaccgtggaggaaagcgcaagcgaggacgacaatgacagccgtcaaagcctgtccgtagacgatgtgggggcagaggtagatgtagaggtggaaggggcatacggtggtgttgcatcggataatttactatgtcatgaaagtatggatgatcccgagtacagcggtgccagcgtcgggagtcgccctgacgggtatgatgaaagggcaccctggaaatgcacaatatgtgggagacctgtcactccacaagagctagctaccttcggggtagtcaatccctggaataagcagggagtgtgcaccgtgtgctttcacgggcaacaagagaggttcaacagcatctggggttaatggacttcattaccattgaagctgaagataccaatagttcagacagcagccaatgtgaggaagaggaagataaggacgattctgcattagggacatttatagacgatagtaatggaaacgacgaacccgacaatatcagccatgtgcaattgttgcactcccagcgatcacaagactttgagattatacctaagcgagtggcaggacattatacccgtaagcgacgaagaacacggtccccccacggtgagagcgatatcattcgatgccagcacggcggacatggagacccaaagaaaactgtgcaagtattgtctcatagtcctcctaaaacgattaggcagtattttacgtctggggatactaatagaccccataaagatacttcggagggatgcacagggtcaggggccacgcacaccacacgggttattaccgcgcaggtgcacgttcctgctgaacctacgtatacggctggaggaaattctcctgccagaactccccttagacagattgatcctaatggtcgacctccaggacgtgtcacacctgtatcgcctcctgtcccccaccgtagacgaaacgtatctgcaggtgggacacattatggagagaatggagcacgcctgccgccgccgtatgaaacggcatcagaaacgaggcagactgaaggacagaaactacttcaacgctgcctagtttcaaaaaataagacattaactgcgttagcggtgtttaaagaactatatacggctagctttacggaggttacaaggacatttaaaagcgataaaacgcaaagttatgaatgggtgttcatggtgttggggtgttcacatattgcattagaagctgtaaaagatgtattaatacataatacggaacatgtaatccttgatatagatccatataaacatctgggagtatattacgtaggatttacagttagcaaaagcagggaggggcttttgcggtttctaaaacaacataatatatttaccgaaaatgtagtgctatctaatccgccaaataaacgctccgtgctatccgcgctattctttgataagttagtacaggtaagcggggacaaaccacagtggatgatagatataataacctctggggacaagggtggtgaaggctttgaattaagtaaaatgatacaatgggctctggacaacaatatgtacgatgaaggggcaatagcatataactatgcgttattagctgatacggatctgaatgcacaattatggcttaagcataattcgcaggctaaatatgtacgggacgctgctacaatgtgtagacactatagaagggggcaaatgcaagcgataggggtaatggaacatttggctacacgtatgcgggaatatgcagatagcgatatagaagagggctggaaacgtataattgtgttcttacgatatcaacatgtagaccatcatacatttataaatgatttaaaatactggattgtaaatagaccaaaacgtagtacaatcgctatagtgggaataccggacagtgggaaaagcatgttcgggatgtcactcatacaatttctagatggacgggtgttaagcttctctaatcataaatcgcatttctggttacaaccattgtcagaaacacgttatgcattagtggacgatgtaacatggcctgcatgggactatatggatgtgtatatgcgaaatgcattagacggtaaccctatatgtatcgattgtaaacatagagcaccaatacaaacaaagtgtccgccgttgttattaacgagcaattacgaccctagggagcgtgggacaggggcggaaaacagctaccgatacctactaagtagaattacctttatgtcatttaataggagtatcccatgtattgggggacagccgagattcctaatttcaccagcggactggagatcattcatgcttaagttcaggaaagaactggacatcaacctaacagaccttgattatggaggggctacgggagagcctggagagactgcagaggagagaggctgagatactagagcaggacccaactgatctgcagacaataactgaatattgggaaaacgttaagaaacaacacctgctgctatatgcagctggacaaaaaggttataaacaactaggcctgcaacgcgtaccaccactacacgtaagcgaacaggaggcaagggacggtattctgatggtcgtcttattgaggtctctcctaggtacaccacatgccctacgcacgtggagtctaggggagtgggggcccagactgttccggaccccaccggacggtctaaagttcgggccgcatactgtacgggtattttattgtaatgatccaagtacggaaaccgaatacccatactgggatagttatctattttatgatcccactacaggggagtggacagagggtatagggggttatgataacgtaggtatatggcatgaaaccattaatgggcgagggtatcatatgatatggagggatgaagcgcgtagagtgtgtgggggtaatcaagtaacatgggaactttccactagcgaccgtgactcgctagacggtataccgccaccgctactggaatcaacgcgtgtggagtcacccgaaccagccacagagccggagacaccatctccaccacaactgtcaaactacgccttaggcgacccgccaaacatacgaggcggttcaaattcggcgcgcacccgatctcgacgaatccgtacaagaggtacgggtactgacacaccaacaggcatccgtccagaggacgtggggacggcgagaacaacggtccggggtggtgggacgcgactagaccgcctcatcgcggaggctaaggacccacctgggctgtgctttgtcggccggacaggccagctcaagaccatacggtaccgggtgcagaccggaccgtataacgtaacacgcataagcactacctggcactggataggggacggggaacatttgtcacgtatgattattctgttcaatgattcgcatcagcgggaagtatttgcaagagcgtttcgggtcgtatcgggtgtacgggtctataaggtctcattgtccggaatataaggatggttgcgtacagacggttccttctccagctgcctgttcctcctacctcatttccgtattattacgggccgcgttttttattacattctgctgctacctcgtcctctttgtcctctactgtgtccccctcctccgctcgtactatatcgaggcggcggagagccgcagcggacgacctatggcgtaaatgccaatatggggattgtccagacgatgtccgtcaacgttatactcagactactatagctgataaaatactccaatggggaagtgccctagcatatttaggtggccttgcggtcgggacaggccgtggcggtggtggtgttcggttgggtagtggggcgtctgcagtgccccgacccacggcccctgatactacaattccgtttacccgtcctgttgtacccgatagtgcggccgccgttcctgagggtggcatagggactgttcctgctgacagaccattccaggtgcccactgccaacgttcctgtggatgtggtacctgtgtccgggaccgagcatgctaatacattacctcctaatacgtttgtgaatcctgcctttgagggggatttagattcctcatcctctatcgatagtgtagttataggcgatttggttcgtagtgaggatgccccacggacacggggtgacacctttattatggaacatgaatttgtcggccctattagtgaggagcgtaggccgtacttccctactcttaatagtaatcctacacaacctttcgaggaaatcgagatggttactttcggtagtactgctgatgaggattctgcaatagtcggtcgggagcctagcacaagcactcctgtcacttttgggacacgtggcactgcaggacggcctcgtggatacactcttgatgtaaccatcagtaatcccgtatatgatgaggccctagatattgacaggttatttcaagaaggtttgcaggagtgggtggatagtgatattatctctcccgatgcccccggcattcctcttggggatccctcatatgcaacagcgtccttcggaacacggttgcaggtatctagaccgggacagctccctggtatccgcctccgcagtggtagacggttgcagataccggtattgtttacaggggacttgtcgtccatagctcccgatctggaattacagcccttgcagcctgttggtgcaaccggcactgtcgtttctaacagtggtgctgcagaaacagtgttctctgctgcagacacaataggctgggacggtcagaatatttcggcctctactggtattatagacattggtcccgtgtctggggacgacctaccgttttttgaaatcccccttgatgatccctttcctgaaatggagattgtggaggagagtgagactaatactactccttatactttatctgatatctcggttgtggatactacttatccgttccctgctatcacatctgcggcgtacccgtctgtgagcattcaggaggatggggggattgtcgtctacccgactcctgccgcaccggtgtcgtatggtgggctcgtcagtatggaccctaactcgttgttctggtttctgctccgccgccgtcggcgccgccgtcgtaccactaaacgtatccttctcaacagatgagtgctgctgggcctgctcctgcgttaccatcggcattgtatattcctaatgctgcgcctctacaaccacccctatttactacggacgactttgtttcccctacggactatgtgtatcacgtaaatacgggacgtcttttgatggtcggtaacccatacttttctgtccctgatgctgataaggaccgtgcagcggttcctaaggtgtctggtaatcaatatagggtgttcagattgaagcttcctgatcctaatgatcagtttgacctcccggacggtctgtttgacccggagaagtttcgatatgtatggcaacttgtaggccttgaggtttgccgtggtcagcctctgggtgtgggcatttccgcggccccggcctttaataagggtcgtgatgttgaaagccctgcacgtttagttgcggacgatgccacgcgagaggatgacaatcgcgtgagtgtcggtcttgaccccaaacagaaccagatgttaattgtcggttgtgccccagcatatggtcagcactggggcaaggcaactccgtgtccggatgacacattggatacccagtgtccacctatagaactgattagtagcacattgcaggatggtgacatgtgcgatattggcttcgggtgcatggactttgcagccttggccgccaatacgtccgatatacccttggaactcattaacactgttagtaagtacccagactggatccggatgcataatgatcctaagggcgattgctgtttctttctaatgcgtagagaacagttgtatgcacgacacatgtggcaacattctggtggcatcggtgaggccataccgagtgtttatcttaatacctcgtttacgagtactaataactgtgcttacatgtgtgttccttccgggtctgtatacacctctgatacccagttgtttaatcggccgtactggctgtccaaggcgcaaggtcctaacaacggcgtttgttggggtgatgatctgttcattactgtgttggacaatacgcggggtggggtcatgaacatttctacgaaacctacggatagtggggatgtgtataaaccttcggacttccgtgaatatgtccgacatgtagaggaatacgaattatcctgtgtgttacggctatgtaaagtgcccctctccccagatgttcttgcctctctctaccgtgctgtcccccatgtgctgggccgttggggtatttccgagtacccacaggccgatactaccccggaggataaatatcggtatatcagttcacaggcaacccgatgtccactacctgctgcagatacgcctacgccggtgcaggatccgtgggcggatatgactttctggactgtcgattgcacgtcccgcatttcccccgaattaccgcgttttcccctcggtcgcaagttcctagctttgcccggaccccgtccggcaaccccattatatggaaaacgttctgctactgccgcagccctcacgggtgcagctggtgtgcgatctgctggcgtccgctcaggcgtgcgcactgcgaagcgtaggcggaggtaatggcgtcgcttatatgatatccaatgtttttaacgttacataaatatatgcatatcttgttacatgcctgttcctgtattcattgtaccctcgtcaaccccttagttgacagccccttatacgcccacatttcgatgtgttgagacatttcctggggcttatgtttgcgcgttcaaccgttcggttgacattcgttgacagctgcaattgctcgaatgaatagaaggcgcggcggcagcaaccgtctggcaccgttccgacttgaaaaaggtacacccttatataatatatatattatatcatcagatataaagtttttaccactctcaaccctatggttgacataacgtatattggataggaaccgtaaccaatagcggttcgtacaggggggtccttcgcttccggatcccatatacgggagatatcataagaataaatagctgaggggaataacagt

SEQ ID NO: 18 - DNA, 7303 bp, Bovine papillomavirus type 9
aaagtctgcacctggtgctgtgtaaagacacaggtatgcgggcacacctaacgggtgcgcctttgaagtttttattggccataaaactaattgttctggccaagttggctataaacagttcctgaaaggcgcgcacgggcagaaatccactgcaagctgagacaggacgtaccgtttgcggtcggaaccgtctcatcacagtaagttgttataattaacaatcacgtacatgtagttactgactgcacctaattcggttgcaccgttcccggtacgtatataaaggcagcagtttggggtctggtgagaagcatagcttcataatgtccatgcgtctaatctatttcttattgctgttgtggtgtgggttcaactttttgtctttgttgtttgcagttgtgatttatttgcttttattatctgctatggataatcttaatggatgggattgagggaatcgtactgatattattgattttggtgttttggttcgggtttacttttgttgcctctgtcctcattattgtgatatatgcctgtctaatatttgcaatagaaagtatgaatggatggaactgaagctgtgctgatagctctattgatctgtgtaatttgtgatattatactattgtatgtagctgaaattctgcagcatttgcttatattgtctttgctagatgaggagttttagctaggtgtgggcataaaagctgaccgtaccagtacttaattgcaggtggcgcctgagaggagtcacataaaggtcttacacgttctgcaaggaaaatgaagggccactacgtgactttaaaggatattgctttagaatttgaggatgcagttagcccggttaatttagattgtgaggaggaggagatagagacagaagttgtggagtgtcctaacccttactccgtcacagcaacttgttatgtttgtgaaaagacattacgaatagctgttgtcacatctgcggacggcatccgtcaactgcaacttctcctgttggattcactttccctactgtgtgcagcttgttgtagcgacgcaatccgcgctggcagaccccgaaatggaccctaaaggtattgacgtccttgagtttattgaggaacaagcagaatgtagtgaatctgatagccaggaggggtgtgaagaaggagaaagcttgtcagatttgtctgacttaattgataatgctgattgcgagcaagggaattccagagaattgtttgcacaacaggaggctttcgatttccataaggacatacgcgctgcaaaacgaaagttaaaacggtcactccgaaagcctttacaatgcataacgagtcagactaacaacagccctcgtagagctgcgtccaaaaggcgattgctagacgacagtggttataatgaagatattcctggagaggtggctcctcaggtagatgaaaatggcgcatcagagggatacgggagtctagcttcgcaaaatgtgtcggggcagaacataaatgcttgtaacaaggagaatggggattgcagagcgtttttgcgagcaggaagccaaaaagccgcctatttggcaatatttaaagagaagtttacgatcagttttactgcgttaactagaatttttaaaaatgataagacgtgctgtaatagttgggtgggggtggtgtttggtgctagggatgaacgtatggaggcttcaaaaaccattttgcaaaactgttgtgactttgtgcttctgcttacacatacctgtaaatatgggtttatgggattgtacttgttgtcttttaaaaatagtaaaagtcgggagactgtcagacatttatttaaacagcttttgcagatggaaaaagatgaaatgtttttggaaccaccaaaattacgaagtctgcctgctgcaacgttttggtggaagatgcagcacagtcagggctgctatgtatgggggcaattgcctgaatggatagcaaggcaaacaatgatatctcatcaaatagcagacgatgagccttttaatcttagtcaaatggtgcaatgggcttatgatcacgattatgttgatgaagccaaaatagcttactactatgctcgtctagcaactgaagatgccaatgctgcagcttttctgcgctgtaataatcaagtaaggcatgttaaggaatgtgcacagatgacaaggtactataaaactgctgaaatgagggaaatgtctatagggcagtggattagaaaaagtatttcagcaatagatggtacaggcgattggaagactattgtcaactttttaaaatatcaacatgttaatttcctgtcatttcttagtgcatttaaagacctgcttcatggtgttccaaaaagaaactgcatggtgctttttggacctcctaacactggcaaatctatgtttattatgagcttaatgaagactttgaagggcagagtattgtcttttgtaaattctaaaagtcatttttggctgcaacctttggactctgcaaaaattgctgttttagacgatgctacacgagccacatggtcgtactttgatacatacctcaggaatggtctggatgggacccctgtttcactagatatgaaacacagggctcccttgcaaatctgctttccccccttgctaataactactaatctcgatattatgaaggaccccacatatatgtatttgcatagcagattagtggcctttgagtttgctaaccccttcccattagatgaggctggcaaccctttatttttaatcaatgaactcagctggaaatctttttttgaaaggctttggactcagctagagctaactggccctgaggacgacgaagatggacagcctagaagcccgtttcgagtctgtgcaagaacagctgttacagatatatgaaacagattcacagacattagaagtggcaatcacgtattggtcacttataaggcgagagcatgccttgtattatcgagccaggcaagaaggtaaaacaaggctgggactgtatccagttcccccctcaagggtgtcagaaaaaaaggctaaggatgcgatcaagatatatttgcatttgcagagcctgcagcagtctgagtttgcaaatttaaagtggtcacttgtggacactagcttagagaactttctagctgcccctgaaaacacattcaaaaaaaaagggcagcttgtaactgtggtgtatgactctgatgctaataattccatggtgtatacagcatggcgagaaatttactatgttgacgaaaaggacacatggagacgaacgcacagtcaggtggaccatgatggaatattttatgaggacgcacaggggaacaaagtgtactatgtgaactttcatgatgatgcagcactgtattctaatctagggcgatgggaagtgcattttgaaaaccacgttctttctccccctgtcaccagctcggtttccggtgggcctgccaagcaccgacgaccccaggccggggatcactccccgggacacacctctgccgtttctgccgcagccgggcaatataccggagactccgggcaacggcacacgcggtcgaggtcgcggtctaggtccaggtccagatccaggacgcctacgaggccgacatccggaggaccccctcgaaaacgagggagaggaggaggaacgcacaccggaggatcacccaccgaggtacggcgacaccctggaggaacgcctccgccaactgctgaacaagtgggagtccgacatcgaacgcctgagagaaaaactgcgtctcgccttactcaccttatagaagaggccttcgaccctcccatcctgcttttgcaaggtgctgcaaatacccttaagtgctttagacgacgtgccacacattcccatcctcatagattcctgtgcatgagcacaagctggacatgggctagcaaaacgtctaccttaaagtcaggccacagaatgctagtggctttttcgaactttgaccagagaacgaactttctagccaatgtgcacttgcccaagggtgtaaccgctgttacggggtctttagacgggttatagcatttattaaaatggttcgtgcagcaagacggaaacgtgcgtctgaggatgatttgtacaggggctgccagatgggtcaggactgccctatcgacatcaaaaacaaatatgaacacaacacacttgctgacaggatccttaaatgggtcagctcatttctgtactttggcaccctgggcataagcagtggcaaaggcacagggggcacaacaggctacactccattaggaacagggagtgggggagtacgacctggtaaaggggcgaatgtagttcgcccaactgtcattgttgatgcgctgaatcctcccggcgtccccattgaccctgctgtcccagacagcagtgtagtacctctgctggaaagcagtggaggaagcaccacacttgacacaccaatgggtggggaggtggaaatcattgctgaagttcaccctcctcctactgtgggccctcctgacattataattgaccatccagatgaccctcctatattagatgtaacctctgaaacacatcctacgtcttctgtaaaaagcaccactagcaagcatgacaacccagcattcacagcatatgttgctagtgcacaattgcctggagaaacttctgcagcagatcatgtctatgttttgcatggtttcaatggtgattttgtaggccctgcagattcagagggcaatgcaatctttgaggagataccattagacgagtttggtgtccctgatagtcccccaagcagcagcaccccagacagcagatttaggagggtcctaaacagattccaaagaaggctgtataacaggaggcttgtgcagcaagtcaagattactgacagaaatacatttttaagacagccttcccaattcgtgcaatgggaatttgataaccctgtttatgaagatgagtcgttgtcattaatctttcaacaagatgtggacgacgtgtcagctgcacctgttgcagaatttcaagacgttgtcaaattaagcaggcctattttcacagaggcacaaggcttagtcagagtcagtagactgggacagcgtggcaccattcgaacccgtagtggcttgcagataggtggacacgttcattactacactgatataagtccaattagacctgcagaagacatagaaatgacatcctttggtgaagtctcaggggacagtattattatgcagccactagcagaaagtaccttaataggtgcaggcgttagggaaaacatggatgaagggttaatagaatattctgacagtgttttagacgatgatcttaatgaggatttcagcaatgtcaggcttgaaatcaccacgtctgcacgcaatagaaccagtattctcactgttcaagatggaataccccctgggtcagtcaaaatgtttatcaatgataatgcagctacagttcatccctattacccagcacatgaggagggtacagacactagggacattccattgtctcctacactacctccagcaataattattgattttgatgaggacacagcaactttctttttacatcccagcttgctcaaaaaacacaaacataaacactggttcttttaatgttttgcagatgtctttctggctacctagctcgggaaagctgtacttaccaccacctacaccagtcacacagtttctagacacggatgattttgttacacgaacagacatctattatcacacgaacactgaaagattattaactgttggacacccctactttgaccttaaacaaggcaacgaggttgtggtgcctaaagtgtctggaagccagtttagggtctttaggctaaagtttccagaccctaacaaatttagcttccagaatccagatgtatacaatcctgacagtcaaagactagtttgggccctacggggcattgaaatttgtaggggccagcctttaggaataggtgtcacaggccaccctgctttcaataagttcaaagatgcagaaaattctaacacaaatccagcccaaggtaaagatgacagggtgaatgtttgtctggaccctaagcaggttcagcttttcattgtaggatgcactccttgtgatggggaacattgggacgttgcaaaggcatgcacagatctcaagcctggcgattgtcctccactagagctagtaaattctaccatacaagatggagagatgtgtgatacagggtttggcaatatgaactttagcacactgcaaaagagcaaatcgggagcacccttggacataatcaatcaagttgttaaatacccagattttctaaaaatgggcagtgacccttatggtaactccatgtttttttatgctaaacgagaacaaatgtatgtgaggcatctatggtccagggcaggcacattaggtgatgacattcctgcaaatggagacaccaacccctattttctgtctggcgattcggctttacctacctcagtatactttgggagccctagtgggtcattagtttccagtgatcagcaaatttataatcgtccgttttggattcaaagagcccaaggcagcaacaatgggatgtgttggaataatgagctttttgtcactgctgtggacagtacacgaggaactaacttcacaatttccgtgcacaccagtcagcctgagcccctgccccaggaccgatatgcagctaccaaatttaagcattatctcagacatgttgaagaatgggaactttcacttatacttcagctgtgtgttgttgatctcaaacctgaagccttagcccatttgcacagcatgaatccctctattatagacaattggaatttaggtttcatacagcctcccaataacattgaagaccagtacagattcataaattcactagccaccagatgccctaaggctgcagacgtgcaggaaaaaatagacccctataaaggaatgcggttctgggatgtggacctaacagagaggttctccctaaatttagagcagcattctctaggcagaaaattcctatttcaaattggaaaaagagctcccaaacggtctgcaccgaaatcggtcgctttttcaagttccagcaaaaaggcgccaaagcgtaggcggaaaaatgtctaggcgccaatccccgaggcactgtgatactgatggttgtgctgattgttgttatgtttcctgcatgaatactcaaaataaaatgaagatttactgatattcggtgtttgtcagttgtttttcactcctatctttgtttgtgcaagaaa

SEQ ID NO: 19 - DNA, 7610 bp, Equus caballus papillomavirus type 1
gccagcggcgccacagcaccacaaatggcgcggccgcgacctatgctagggaaactatgtatgtggtgccatgagccactgaccaatctggacgcactgaactttcttgagtgtaacctctctatggtctggaggaacgggacgccatacggcgcgtgtaaggcgtgtgtggagttccagtgctttctggaggtctatctacacagcgaaggcgactataggccccgtgaggtagcgcaggaggtgggacggtccctctgtgatgtgcgagtacggtgctggtcctgcagcaagcccctgacgaagaacgagaagcaggaaatagagctgctggggaagccattaacgaaggtgcgccacaaggagtggagagctctgtgctacaactgcggtctgccacgacatgatagggaatggctccccgtcattgagggagattgttctatctgagctaccgcaaagcctggcagacccagcagaagcggaaagcgaagaggaggaggtggaggtggaattggacgccgtccgacctcaggcgccatacgccgtgtgtaccgtctgctgccgctgcggagaaaaagttggactttgtgtgcttgcaaccgacgagggaatacacggcttagaagagcttctctttgaggctctgcagcttttctgcgcccagtgtgccccccccatcggtcgccatggacgctgaggaagcaggtacgcctgtgagagatgcaggtacccctgtcagggaaggctcctcatggtttctgcagcaagaggcagactgtagtgatatggacggtagcgaggccagtgaggaaagtgaggccgaggatctaatagacgatgctcccgttagacagggaaattccctgctgctattccagcagcaagaggcgcaggcggacgagcaacacctgtcggtgtttaaaaggaagtactgtagtcctaaagagaaagtagcagatctcagcccgcgactgggagctattagcatttccccgatcagggggccccaggttaagcgccgcctgttcaacccagagcaggacagcgggttagacctctcgctgcagaatgaagctgtcgatgttgttgagccgacggagaaccaggtaccctcggatgcagttcggacagttcgccacgtggatggacagactggggggcttaaccttaatatactgcgaagcgccaacagaaaggctaccatgctggggcttttcaaagatgcatttggcgtgccttatggggagctaacaaggcaattccgtagtgataagacggggtgctttgactgggtggtggccgcttacgccgtgcgggagccattttttgaaagtgggaaagcgcaactaaggcagcactgccgatatacgcatgtaacctataggcccatgccacggggcactgtgctgcttatgcttgtgtcatttaataaccaaaagtgccgcgatactgtgaataagctaatcaggaccctgtttaatgtgcatgagcttctgctaatgctggagcccccaaagattcgcagtgtggcggcggctatgtattggtacaagcagtcattgacgaatgctacagaaacctttggggagcttccagagtggatcaaaaaattaattcttataaaccaccagacggatgaggaagtgaaatttgatttttcacagtttgtgcagtgggcatatgataatgaatatcaggaggagcatgaaatagcttataattatgccagcatagcggatgaggacagcaatgctgctgcatggctaggcctaacggggcaggcaaaggttgtgaaggatgtggccacaatggtgcgctactacagaagggcagaaatgaatagaatgtctatgtctaattggatacacaatagaaagaaaaaatctaagccagggcaatggcagcctattgtaaactttttgaagtatcagggggtggccatggtaacatttatcaatgcactcaaatcttttttgaagggcacaccaaagaaaaattgtttggtgatatgggggccacctaacacaggaaagtcgtggttctgcatgagccttatgcacttcctggggggtagggtcctgtcacatgtgaactcgaacagccatttttggctgcagcctctaggggacgctaaagtggcactgctggatgatgccacgactgtggtctgggactactttgaccggtacatgagaaacgcatgtgacggcaaccctatatctttggacatgaaacacaaggcccctgttcaaataaagtgcccccctctacttattacctcaaatatagatgtcaaggcagatgataggtggctatatttgcacagccgcctggtcaccttccatttcccaaatttgtttccctttgaggacgatgggagtcctgtttaccaatttaatgacgaaaactggaactctctttttacaaggttatggagagcattagacctcagcgaccaagaggacgagggtgatgatggagaccctgcgccagcgtttagatgctgtacaagaaaaactaatgaatcttttggaggagggcagctctgatctgtcctcccaaatttgctattggcaggcggtgcgaaaggagaatgtactgctgtactacgccagggagaagggcctaagcagactgggattacaaatggtaccccataaggctgtcagccaatcacaggcaaagcaggccatacacatggagctaatactgttgagtctgcagggctcctcctatgagcaggaaccgtggacactgtcggactgtagctgggaacgctggctgcaggcccctataaactgtctaaagaaggaccctgtaattgtagaggttgtgtatgatgggaactctgaaaatgcaaactggtatactttgtggggattgatatactatcagacctttgagggggactggatgtgtaccagggggcagtgtgaccattcgggcctttattatgaggaggaaggccataaaaggtattatgtgcacttcatagatgatgctgccaggtattcaaagactcggacctgggaggtacgatgcagaaaccaaatttatctcccttctattcctgtaaccagcactccgcctcagtctccatctcacatcgacctccctgacggagcagcaggaggaggacctaatcaatcgcctcggcctggagccttggcagtgtcgcctcaggagcccccaaaaaagaggtaccgctctccagctgacacagtcagcagctctcggctgtcaggggggctccgctgcccagccgactggtgccgcaggaagctacaacgcacgtctgcccccacctgggtgccgccgtcggtatcggaagtacctgaagcgccggagggatcagtatcggagactgggggagcatctccaggagttgattcaacaactggacgggggaacgaccccgcgcccgtaccattggaggctgcgttcgccccaatagtcatctttcaagggggtacgaatcaatgcaagtgctataggtggaggttaaaaaaaaggcatcgctctctttttgtggcaattaccactacttatttctggaccggggacaaagggggacaaagggttgggaatgcacgtttaatggttacattttcctctgatttgcaaagacgtctgctgcttgccactgtgccccctccccgaggtgtcacggctacctcctttaccctaaccccctcctgactatgccccttccccaacccgcgtgtgtttgcatttgtaccccatcaataaacaataaagaccatgtcagccagccacggggttctgacggtcccacggcggacaagggtgaggcgggcggtgagaaggccgagggcctcggtgcaagatctgtaccccacctgtcgcacgggggactgccccccggacgttgtaaacaaggtggagggtacaacccttgcagacaagcttctccaatggttaagctccttcatatacctgggaaaccttggaatagggaccgggagggggggtggcgggcggttcgggtatacacctgtgggtcgccccagcgggccggagggtggcgttcgtgtggcgcgccccagcattactatagaccccttgggggctgcggatgtaatccccctggacacccttgggccggatgctcctgcgattgtccccctttccgaggtggtggagaccgatttgggtgcaggggctggtggcctgggggaggtgcccgagaccgagcttacttctgggggggccccggtgctagacaccacccatacatggctgccccccggctccgaaggggggtttacagctacattcccgaacccttcatttgatggggatgtaataagtggtccctcaaacagtgatcctgtggtccggggggatgtatttgtcacaggggatatggatgtgtccgtgggccgggaagagtgggagttggacattttttctggtcccggaccgtctaccagcacccctgacccaagtgtgcgtgtctcagcccgcaccaggggaggcctgaccgggagacaatatgaacagatagaactgcaagatctcgcggcactggggggcggggggaacagagagtcctatgcctttgacaatcctgtgtttgatagcgggtctgtggagtttgcagtggactaccatggagatcccccgtttcaagacctgcagaagctgggtcctgtggaaacatataggtcctccagaggggtttctgtgtcccgtgtaggtcataggggcacgatgagcacccgatccggccgtaacataggggcacaggtccactattttcacatcttaagcagcattgcgcctgaggagtcgctggtgtccggccctgtatcggggcgtcctggggaggacgcgtttgaggagattagcttgaccagcttccctagcctgtacagtgagtcagagctactcgatgaggaggtgatagaaccatccggccatctagtcattggtagtgggcgcgagtcacgtccttatccaacagaagtaatgtacaggccgcctgtggtgacctttgacctttcttttgagcagggcattgaacctgcagtgtatatgacccctaagcctccccacggtagcattcctgggattattattttggttgatagtccagacacctcgggtgtctttgacctgcacccctccttgctgcgccgccgtaagcggaggtatatgtggaactaatttttttttttcagatcatggcgtcctactggtcctccaactcacagaaggtgtacctgcccccaaccacattgactaaggccgtatccacggacacctatgtgaccagattgggtatatactatcatggtcacagtgaccggcttctgacggtgggccacccattctacgagataacaaatgggcgtgaccaaaccatgcgggtgcccaaggtgtctgcaaatcagtttcgggttttcagagtgattcttccaaaccccaacaaatttgctcttcctgatagcaatgtttttgatccagattcagaaaggctggtgtgggctgtcaaggctatggagatttgcagaggtcagcccatagggcctcaggtgactgggcacccattgtttaataggtttgaagatgtggaaaaccctgcagtttataagccagggttcggcacgggtgacaagagacagaatatggcatcagactataagcaaatacagatggtggtactgggctgcaggcctgccttgggggagcactggggtaagacacgcagcatttgcccaggcattcaaaataatgttctcaccggtgactgccctgctatagagctgtttcacaccactatagaggatggagatatggtagacataggtctagggaatctggactttgcacagctgcaggccgataagtcaggtgcccccctggatatagttcagtcaatttgcaaatatccggatacattgaaaatggcccaggagattactggtgacaccatgttttttagtgctaggcgggagcagagctatcttagacacatgatgacacgtgccggtatcaacaaagaggctataccagaggccctatatataaagggcgccacagagccacagaatactgtgggcacctctgtttactgtggggtggtgtctggctccttatttagtagcgatgcacagatttttaacaggccattctggctaaatcaggcccagggtctaaataatggcatagcctggaataatcagctttttgtcacggcggtggataatacccgggccactaatttcactataaccgtggcgacagatgagagggagaaggatacctatgatgctggaagctttaatgcttacctgagacatgtagaatcctatgagctgcagtttgtatttgaactttgtaaggtcaagctgacccctgagaacctgacaatcctgcaccagcaggaccccggcatacttaagggctgggagctgggggtgacccccccttcgggttcggtcttggaggacacctaccgctacattaattctgtagcgaccaagtgcccgcctaatccacctgaggaggtgcaggaggatccctggggacggttcgcattttggagagttgacctttcagagcgcttttctcttgaccttgaccagtttccattgggaagaaggtttttggccctttccgcaccccgtacccgcacgtccgcagctaagcgtaagaccccggtttctgccaagtcttctaaacaaagaagaaagggctaatcccttgttttgcatatattcctgctcgttcttgcctgatgtaatcgatataagcctttttgcagggtgctcacacgcaactctcattgtttgtacagtgttttttatagctgtagtgtgttgtaagcatcaatgtgctatgtctaattgtataataaagggtgagtgtcctgtgactgtcccttagtgtccttgctatttgtccttccttcacggaccgcccaaaggaatttgggcggcacttgagcgcctgccggctcttcccgcgcgctgcttggcttttgtgccaagttggcgccaaattcaaacaaaaacaagctgctagctgccaagaaacgctggccggacgcccaaacacgctgcgacgggtctctaactgcctccagcacttcttggtgacgccataaggaggagcaccgaggtaggtgctttgtcagcttttttggcttgtattcactggcaacccggcgagttgctgtgtgagaacttggcagtagaacaggggctatggttttggcacgggtacggatggggtcgcctgaaggcgtcgcagtactcaggacggtgctggcgcacctggccgggacctccgccagataccgcctgtggctgctagaggaactggcgggtatcctatacggtgagtaagacctcgggggtctgtgccaggtgcttaatccgggtgctgggacgttttcccagaccggcaccgctcaagggataggcgtcccaatctagctttcagcagcaccgctgacgggggggctaggaccagttgcaaccggcatcggtgctctgccaatttttaaataagcttgttgttgttgtctacaacaagcgcccgaatagtaatgttcccgcttcggggtcctataaaaatga

SEQ ID NO: 20 - protein, 531 aa, Human papillomavirus type 16
MQVTFIYILVITCYENDVNVYHIFFQMSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVHTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTQAIACQKHTPPAPKEDDPLKKYTFWEVNLKEKFSADLDQFPLGRKFLLQAGLKAKPKFTLGKRKATPTTSSTSTTAKRKKRKL

SEQ ID NO: 21 - protein, 516 aa, Human papillomavirus type 5
MAVWHSANGKVYLPPSTPVARVQSTDEYIQRTNIYYHAFSDRLLTVGHPYFNVYNINGDKLEVPKVSGNQHRVFRLKLPDPNRFALPDMSVYNPDKERLVWACRGLEIGRGQPLGVRSTGHPYFNKVKDTENSNAYITFSKDDRQDTSFDPKQIQMFIVGCTPCIGEHWDKAVPCAENDQQTGLCPPIELKNTYIQDGDMADIGFGNMNFKALQDSRSDVSLDIVNETCKYPDFLKMQNDIYGDACFFYARREQCYARHFFVRGGKTGDDIPRAQIDNGTYKNQFYIPGADGQAQKTIGNSMYFPTVSGSLVSSDAQLFNRPFWLQRAQGHNNGILWANQMFITVVDNTRNTNFSISVYNQAGALKDVADYNADQFREYQRHVEEYEISLILQLCKVPLKAQVLAQINAMNSSLLEDWQLGFVPTPDNPIQDTYRYIDSLATRCPDKNPPKEKEDPYKGLHFWDVDLTERLSLDLDQYSLGRKFLFQAGLQQTTVNGTKAVSYKGSNRGTKRKRKN

SEQ ID NO: 22 - protein, 513 aa, Deer papillomavirus
MAFWQPGQALYLPPTPVTKVLCSEQYINVRDIFYHGETERMLTSGSILSLEVTQKHTTVPKVSPNQYRVFRVALPDPNQFALPDKALHNPSKERLVWAVVGVQVSRGQPLGGEVRGHSYFNTFLDAENVSKKVTAQGTDDRKQAGMDTKQQQVLMLGCTPAIGEYWTKARPCVTDRPDAGSCPPIELKLSFIEDGDMMDIGFGAANFKELNATKSDLPLDIANSICLYPDYLKMTEEAAGNSMFFFARKEQVYVRHIWTPWGTDKELPPEAYYLKPPGEMELKMPSVFFASPSGSLVSTDGQLFNRPYWILRAQGMNNGVCWNNTLFVTVGDNTRGSTLTITVPNNDEPLTEYDTSKFNVYQRHVEEFKLAFILELCSVELTPETVSSLQGSMPSILENWEINLQPPTSSVLEDIYRFIDSPATKCADNVSPSKPEDPYSAHKFWEVNLKEKLSLDLDQFPLGRLVLQFDCRLDRLLPQKDHFTYPEKRYKRHMRITGTVRKVLLYICFSLNS

SEQ ID NO: 23 - protein, 494 aa, Bovine papillomavirus type 5
MAVWQQQGQRLYFPPNPVTKVLCTESYVKRTSIFYHGETERLLTVGHPYWKIPEQNIPKVSGNQYRVFRVQLPDPNQFALPDKNLHNPAKERLVWAILGLQVSRGQPLGAPVTGNQLFNVWTDAENVTAKRALPGSDDRKQLGMDVKQTQMLLIGCTPAIGEYWGKAIPCEGKQPKAGDCPPIELKNKPIEDGDMMDIGFGACDWKDFSQNLSDVPLDLINSKSLYPDYLKMAEDSLGNSCFFYARREQVYVRHVYSRGGEQKEAIPKDMTLPQQVPDNKDTSFTFMGTPSGSLVSTDGQLFNRPYWLYQAQGLNNGVCWDNELFITVGDNSRGGVFTISVPVDDRKPEQYNSANMNIYCRHVEEYKLAVILELCSVELTSETVAYLQTVNPSVLEKWEVGVNPPPATVLEDTYRYQESKAIKCIDQTAAAKKDKYENLSFWNIDLREKLSADLDQYPLGRRFLAQNGITCSRKRLRPASTKKSTTNKKRKTSR

SEQ ID NO: 24 - protein, 516 aa, Human papillomavirus type 4
MSSWLSTTGKVYLPPAQPVARVLETDEYITGTSLYFHAGTERLLTVGHPYFPVKDVQEPHKVLVPKVSGSQFRVFRFNLPDPNRFALIDNGFYDSDHERLVWKLRGIEIGRGGPLGIGTTGHPLYNKFGDTENPNGYKKQSDDNRQDVSLDPKQTQMFIIGCTPAIGEHWDKAEPCPSPAPQQGDCPPIELVNSYIQDGDMCDIGFGAFNFKALQADKSSAPLDVIATVCKWPDFLKMGKDIYGDSLFFFGRREQLYARHFFVRAGTMGDALPEPFEATSDYFIGAQNQQDQYTLGPHIYVGTPSGSLVSSESQLFNRPYWLNRAQGTNNGICWDNQLFVTLVDNTHNTNFTISVKSDGANDNYQYKASDFKQYLRHIEEFEMEFIFQLCKVPLTADVMAHLNVMNPNILDNWQLNFVPPPPSGIEDQYRFLQSRATRCPTQTPATEKEDPYKDLSFWVVDLSERFSSELSQFSLGRRFLYQSGLINGSLKRKRIISSSHAQTNTKRSAKRKRSLK

SEQ ID NO: 25 - protein, 530 aa, Mastomys natalensis papillomavirus
MSYIGAIMGRTIIYTRHCPDAGVTAGISIFQMAYWLPNNQKLYLPPAPVQRILSTDEFTTRTDIYYYASSDRLLTVGNPYYPILDGDTVTVPKVSPNQYRVFRCKLPDPNRFAFGEKSVYDPEKQRLAWCIRGVEIARGQPLGIGITGHPLYNRLEDVENPGKYPSAPGTDNRQNVGLDPKQTQMFIVGCVPAQGEHWSRALTCSNQVVKKGDCPPIQRMSGMIEDGDMGDIGYGNLDFRVLQENKSEVPLEVVDSICKYPDYLGMSKETHGNSCFFYARQARLYSRHFFNRAGVQGETVPESLYKKGKDGQAQSTLALATYSGTPSGSLVSSDAVLFNRPYWLERAQGQNNGILWNNDLFVTVLDNTRGTHFSISIATQDENDYTASNYKQYTRHVEEFELEFIFQLVKINLSTEVLAYLHGMDPSILDNWNLTLGPPNDGSLADKYRFIESLATKCPDNVEVTKPDPYKGRIFWNIDLTERLTADLDQFSLGRKFLYQHARISNRKRSLPASRNGGGTSSSSTKRRKK

SEQ ID NO: 26 - protein, 499 aa, Rabbit oral papillomavirus
MAVWLSQQSKFYVPPQPITKILSTDEYVSRTNIFYHASTDRLLTVGHPYYELEKGGTVVVPKVSPNQYRVFRVRLPDPNKFAFNDKQLYDPEKERLVWAVRGVEVGRGQPLGVNVTGNPLFNRYDDVENSSRYNSGHNNDQDNRQNIAFDPKQTQLFILGCVPATGEHWTQAQRCAGAGYEQGDCPPIELINTVIEDGDMSDIGLGAMDHRLLQVSKAEVPMELVNSVSKYPDYIKMLKDPFGDSLFFYARGEQMYARHFFSRAGDDKENPTDTLITGKGNQSTVSTDNYMVTPSGSLVSSDSQVFNRAYWLQRAQGMNNGICWNNQMFVTIVDNTRGTVMNIVTKANGNGAVDTWANNAFKSYLRHVEEFELQFIVQLCKVRLSPENLAFLHKMQPSIIDNWQLSITAPATSNLEDQYRFIQSLATKCPPVEPPQEDTDPYKNYKFWDVDLSEKMSDQLDQFPLGRKFLNQSGLGQNRQVKTAAPTTSMRGLKRKRRI

SEQ ID NO: 27 - protein, 503 aa, Canine oral papillomavirus
MAVWLPAQNKFYLPPQPSTKVLSTDEYVSRTNIFYHASSERLLTVGHPFYEIYKEERSEEVIVPKVSPNQYRVFRLLLPDPNNFAFGDKSLFDPEKERLVWGLRGLEIGRGQPLGISVTGHPTFDRYNDVENPNKNLAGHGGGTDSRVNMGLDPKQTQMFMIGCKPALGEHWSLTRWCTGQVHTAGQCPPIELRNTTIEDGDMVDIGFGAMDFKALQHYKSGVPIDIVNSACKYPDYLKMANEPYGDRCFFFVRREQLYARHIMSRSGTQGLEPVPKDTYATREDNNIGTTNYFSTPSGSLVSSEGQLFNRPYWIQRSQGKNNGIAWGNQLFLTVVDNTRGTPLTINIGQQDKPEEGNYVPSSYRTYLRHVEEYEVSIIVQLCKVKLSPENLAIIHTMDPNIIEDWHLNVTPPSGTLDDTYRYINSLATKCPTNIPPKTNVDPFADFKFWEVDLKDKMTEQLDQTPLGRKFLFQTNVLRPRSVKVRSTSHVSVKRKAVKRKRK

SEQ ID NO: 28 - protein, 507 aa, Human papillomavirus type 63
MAVWLPAQNKFYLPTQPITKILSSDDYVSRTNIFYHATSDRLLIVGHPLYEVTRANDNTMTVPKVSPNQYRVFRVRFPDPNRFAFGDKDIFDPETERLVWGLRGIEIGRGQPLGVGISGNPLLNRFDDAENPSRYNNTHATGDNRQNVAFDAKQTQMFLIGCTPATGEHWSIARRCAGTQFQLGDCPPIELVNTVIEDGDMFDIGLGAMDFGSLQANKADAPLDIAGTVCKYPDYIKMGQEVHGNSLFFFARREQMYLRHVFTHAGIVSEKEKVPTSAYIAAKAEQPQNTIATDNYFVAPSGSLVSSDVQIFNRPYWLQRSQGQNNGICWRNELFVTVADNTRGTTMNINVLNKATPETYDSADYNEYTRHVEEYELSFIVQLCKVKLTPENLAFLHNMDPTIIDSWQLTVSQPPANAIEDKYRFIESLATKCPDNVPPPTPTDPYKDLRFWDVDLSERMSEQLDQFPLGRKFLYQSGLAQRSVPKTVNFRKRRSSNTTVAKRRRRA

SEQ ID NO: 29 - protein, 583 aa, Human papillomavirus type 41
MTGLQYLFLAMMALTLSILLAQQPPPHSCLHSPAMCPTLLLTCIVEVWIMIYILACCAGNVKNANVFIFQMAVWLPGPNRFYLPPQPIQRTLNTEEYVRRTSTFLHAATDRLLTVGHPFYNITNADGKEVVPKVSSNQFRAFRVRFPNPNTFAFCDKSLFNPDKERLVWGIRGIEVSRGQPLGIGVTGNPFFNKFDDAENPYNGINKNNITDQGSDSRLSIAFDPKQTQLLIVGAKPAKGEYWDVAATCENPPLTKADDKCPALELKSSYIEDADMSDIGLGNLNFSTLQRNKSDAPLDIVDSICKYPDYLQMIEELYGDHMFFYVRREALYARHIMQHAGKMDAEQFPTSLYIDSSVEGEKLNSLQRTDRYFMTPSGSLVATEQQLFNRPFWLQRSQGHNNGILWHNEAFVTLVDTTRGTNFTISVPEGDASSYNNSKFFEFLRHTEEFQLAFILQLCKVDLTPENLAYIHTMDPSIIEDWHLAVTSPPNSVLEDHYRYILSIATKCPSKDADDTSTDPYKDLKFWEVDLRDRMTEQLDQTPLGRKFLFQTGITQSSSNKRVSTQSTALTTYRRPTKRRRKA

SEQ ID NO: 30 - protein, 524 aa, Phocoena spinipinnis papillomavirus
MASTSYWLPSTDKLFLPPPAPVSKILSTDAFVTRLDIFYHAGTGRQLLVGHPYFDVLGENDKLIAKKVSGNQYRAARFTLPDPNRFALQDPTIYDPDRERLVWACRGLQVGRGLPLGGGTTGHPYYNKAKDTENPNSGKYPKTGEGDNRQNVSFDPKQVQMVFVGCSPCVGEHWDKVTSTCADQVHKEGDCPAIELVSSHIQDGDMCDIGFGAINNKTLQESRSEVPLDIVSSICKHPDILQMSNDPFGNSMWFFAKREQMYVRHMWARRGTVSEKVPDPANGGAHAHEFYLSPKNAEEKAMASTIYSATPSGSLITSDGQLFNRPYWIQTAQGKNNGICWGNEVFVTVADNTRSTNITISVKDPSKNNAHQGAYEADHFKIYTRHMEEYEFSFIFQLCKVPLTPEVLAQLNNMNSKIIEKWNVGFATAAPASSSLAEHYRYINSLATKCPPAPEDTEEKDPYEGESYWNIDLSEAFSSELDSFPLGRKFLYQASKSIRAPSRSTTKRPAAKSPVKHSSKRARR

SEQ ID NO: 31 - protein, 520 aa, Psittacus erithacus timneh papillomavirus
MSAAGPAPALPSALYIPNAAPLQPPLFTTDDFVSPTDYVYHVNTGRLLMVGNPYFSVPDADKDRAAVPKVSGNQYRVFRLKLPDPNDQFDLPDGLFDPEKFRYVWQLVGLEVCRGQPLGVGISAAPAFNKGRDVESPARLVADDATREDDNRVSVGLDPKQNQMLIVGCAPAYGQHWGKATPCPDDTLDTQCPPIELISSTLQDGDMCDIGFGCMDFAALAANTSDIPLELINTVSKYPDWIRMHNDPKGDCCFFLMRREQLYARHMWQHSGGIGEAIPSVYLNTSFTSTNNCAYMCVPSGSVYTSDTQLFNRPYWLSKAQGPNNGVCWGDDLFITVLDNTRGGVMNISTKPTDSGDVYKPSDFREYVRHVEEYELSCVLRLCKVPLSPDVLASLYRAVPHVLGRWGISEYPQADTTPEDKYRYISSQATRCPLPAADTPTPVQDPWADMTFWTVDCTSRISPELPRFPLGRKFLALPGPRPATPLYGKRSATAAALTGAAGVRSAGVRSGVRTAKRRRR

SEQ ID NO: 32 - protein, 530 aa, Bovine papillomavirus type 9
MRTQQLSFYIPACSKNTNINTGSFNVLQMSFWLPSSGKLYLPPPTPVTQFLDTDDFVTRTDIYYHTNTERLLTVGHPYFDLKQGNEVVVPKVSGSQFRVFRLKFPDPNKFSFQNPDVYNPDSQRLVWALRGIEICRGQPLGIGVTGHPAFNKFKDAENSNTNPAQGKDDRVNVCLDPKQVQLFIVGCTPCDGEHWDVAKACTDLKPGDCPPLELVNSTIQDGEMCDTGFGNMNFSTLQKSKSGAPLDIINQVVKYPDFLKMGSDPYGNSMFFYAKREQMYVRHLWSRAGTLGDDIPANGDTNPYFLSGDSALPTSVYFGSPSGSLVSSDQQIYNRPFWIQRAQGSNNGMCWNNELFVTAVDSTRGTNFTISVHTSQPEPLPQDRYAATKFKHYLRHVEEWELSLILQLCVVDLKPEALAHLHSMNPSIIDNWNLGFIQPPNNIEDQYRFINSLATRCPKAADVQEKIDPYKGMRFWDVDLTERFSLNLEQHSLGRKFLFQIGKRAPKRSAPKSVAFSSSSKKAPKRRRKN

SEQ ID NO: 33 - protein, 505 aa, Equus caballus papillomavirus - 1
MASYWSSNSQKVYLPPTTLTKAVSTDTYVTRLGIYYHGHSDRLLTVGHPFYEITNGRDQTMRVPKVSANQFRVFRVILPNPNKFALPDSNVFDPDSERLVWAVKAMEICRGQPIGPQVTGHPLFNRFEDVENPAVYKPGFGTGDKRQNMASDYKQIQMVVLGCRPALGEHWGKTRSICPGIQNNVLTGDCPAIELFHTTIEDGDMVDIGLGNLDFAQLQADKSGAPLDIVQSICKYPDTLKMAQEITGDTMFFSARREQSYLRHMMTRAGINKEAIPEALYIKGATEPQNTVGTSVYCGVVSGSLFSSDAQIFNRPFWLNQAQGLNNGIAWNNQLFVTAVDNTRATNFTITVATDEREKDTYDAGSFNAYLRHVESYELQFVFELCKVKLTPENLTILHQQDPGILKGWELGVTPPSGSVLEDTYRYINSVATKCPPNPPEEVQEDPWGRFAFWRVDLSERFSLDLDQFPLGRRFLALSAPRTRTSAAKRKTPVSAKSSKQRRKG

SEQ ID NO: 34 - protein, 510 aa, Human papillomavirus type 2
MSCGLNDVNVSTISLQMALWRPNESKVYLPPTPVSKVISTDVYVTRTNVYYHGGSSRLLTVGHPYYSIKKSNNKVAVPKVSGYQYRVFHVKLPDPNKFGLPDADLYDPDTQRLLWACVGVEVGRGQPLGVGVSGHPYYNRLDDTENAHTPDTADDGRENISMDYKQTQLFILGCKPPIGEHWSKGTTCNGSSAAGDCPPLQFTNTTIEDGDMVETGFGALDFATLQSNKSDVPLDICTNTCKYPDYLKMAAEPYGDSMFFSLRREQMFTRHFFNLGGKMGDTIPDELYIKSTSVPTPGSHVYTSTPSGSMVSSEQQLFNKPYWLRRAQGHNNGMCWGNRVFLTVVDTTRSTNVSLCATEASDTNYKATNFKEYLRHMEEYDLQFIFQLCKITLTPEIMAYIHNMDPQLLEDWNFGVPPPPSASLQDTYRYLQSQAITCQKPTPPKTPTDPYASLTFWDVDLSESFSMDLDQFPLGRKFLLQRGAMPTVSRKRAAVSGTTPPTSKRKRVRR

SEQ ID NO: 35 - protein, 504 aa, Human papillomavirus type 3
MALWRSSDNLVYLPPTPVSKVLSTDDYVTRTNIYYYAGSSRLLTVGHPYFAIPKSSNSKMDIPKVSAFQYRVFRVRLPDPNKFGLPDARIYNPDAERLVWACTGVEVGRGLPLGVGLSGHPLYNKLDDTENSNIAHGDIGKDSRDNISVDNKQTQLCIVGCTPPMGEHWGKGTPCKQNASPGDCPPLELITAPIQDGDMVDTGYGAMDFGNLQSNKSDVPLDICQTTCKYPDYLGMAAEPYGDSMFFYLRKEQLFARHFLNRAGMAGDTVPDALYIKGDSQSGGRDKIGSAVYCPTPSGSMVTSETQLFNKPYWLRRAQGHNNGICWANQLFVTVVDTTRSTNMTLCVSTETSATYDATKFKEYLRHGEEYDLQFIFQLCKVTLTPEIMAYLHTMNSTLLEDWNFGLTLPPSTSLEDTYRFLTSSAITCQKDAPPTEKQDPYAKLNFWDVDLKDRFSLDLSQFPLGRKFLMQLGVGTRSSISVRKRSATTTSRTAAAKRKRTKK

SEQ ID NO: 36 - protein, 503 aa, Human papillomavirus type 10
MALWRSSDNLVYLPPTPVSKVLSTDDYVTRTNIYYYAGTSRLLTVGHPYFPIPKSSNNKVDVPKVSAFQYRVFRVRLPDPNKFGLPDARIYNPDAERLVWACTGVEVGRGQPLGVGLSGHPLYNKLEDTENSNIAHGPIGQDSRDNISVDNKQTQLCIIGCTPPMGEHWGKGTPCRNPPAQGDCPPLELITSPIQDGDMVDTGYGAMDFTALQLNKSDVPIDICQSTCKYPDYLGMAAEPYGDSMFFYLRREQLFARHFFNRASAVGDAIPDTFILKSNGGGRDVGSAVYSPTPSGSMVTSEAQLFNKPYWLRRAQGHNNGICWANQLFVTVVDTTRSTNMCLCVPSEASPATTYDATKFKEYLRHGEEYDLQFIFQLCKVTLTPDIMAYLHTMNSSLLEDWNFGLTLPPSTSLEDTYRFLSSSAITCQKDTPPTEKQDPYAKLNFWDVDLKDRFSLDLSQFPLGRKFLLQLGVRSRSAVSVRKRPATSATGSTAAKRKRTKK

SEQ ID NO: 37 - protein, 485 aa, Human papillomavirus type 27
MALWRPNESKVYLPPTPVSKVISTDVYVTRTNVYYHGGSSRLLTVGHPYYSIKKGSNNRLAVPKVSGYQYRVFHVKLPDPNKFGLPDADLYDPDTQRLLWACVGVEVGRGQPLGVGVSGHPYYNRQDDTENAHTLDSAEDGRENISMDYKQTQLFILGCKPSIGEHWSKGTTCNGSSAAGDCPPLQFTNSTIEDGDMVETGFGALDFATLQSNRSDVPLDICTNVCKYPDYLKMAAEPYGDSMFFSLRREQMFTRHFFNRAGKMGDTIPDELYIKSTTISDPGSHVYTSTPSGSMVSSEQQLFNKPYWLRRAQGHNNGMCWGNRIFLTVVDTTRSTNVSLCAAEVSDNTNYKATNFKEYLRHMEEYDLQFIFQLCKITLTPEIMAYIHNMDPQLLEDWNFGVPPPPSASLQDTYRYLQSQAITCQKPTPPKTPTDPYANMTFWDVDLRESFSMDLDQFPLGRKFLLQRGTTPTVSRKRTAVGRGH

SEQ ID NO: 38 - protein, 510 aa, Human papillomavirus type 57
MFCGLNDVNVCTISLQMAMWRPNESKVYLPPTPVSKVLSTDVYVTRTNVYYHGGSSRLLTVGHPYYSIKKSGNNKVSVPKVSGYQYRVFHVKLPDPNKFGLPDANLYDPDTQRLLWACVGVEVGRGQPLGVGISGHPYYNKQDDTENSHNPDAADDGREYISMDYKQTQLFILGCKPPIGEHWSKGTTCSGSSAVGDCPPLQFTNTTIEDGDMVETGFGALDFAALQSNKSDVPLDICTNICKYPDYLKMAADPYGDSMFFSLRREQMFTRHFFNRGGSMGDALPDELYVKSSTVQTPGSYVYTSTPSGSMVSSEQQLFNKPYWLRRAQGHNNGMCWGNRIFLTVVDTTRSTNVSLCATVTTETNYKASNYKEYLRHMEEYDLQFIFQLCKITLTPEIMAYIHNMDARLLEDWNFGVPPPPSASLQDTYRYLQSQAITCQKPTPPKTPTDPYATMTFWDVDLSESFSMDLDQFPLGRKFLLQRGATPTVSRKRAAATAAAPTAKRKKVRR

SEQ ID NO: 39 - protein, 565 aa, Human papillomavirus type 77
MCIYTLAPTLFCLLLHNGLLFLYYLLTQHIMCTLMEAIFICGLLPFLCLGNVAVNVFHIFLQMALWRSSDNLVYLPPTPVSKVISTDDYVTRTNVYYYAGSSRLLTVGHPYFAIPKTSGTKVDVPKVSAFQYRVFRVRLPDPNKFGLPDARIYNPEAERLVWACTGVEVGRGQPLGVGLSGHPLYNKLNDTENSNIAHADNSPDSRDNISVDCKQTQLCILGCTPPMGEYWGKGTPCARTNTTPGDCPPLELMTSYIQDGDMVDTGYGAMDFTALQFNKSDVPLDICQSICKYPDYLGMAADPYGDSMFFFLRREQLFARHFFNRAGDVGDKIPESLYLKGSSGRETPGSAIYSPTPSGSMVTSEAQIFNKPYWLQQAQGHNNGICWGNQVFLTVVDTTRSTNMSLSASTESQTPSTYDATKIKEYLRHGEEYDLQFIFQLCKVTLTPEIMAYIHTMNTALLEDWNFGLTLPPSTSLEDTYRFVTSSAITCQKDVAPTEKQDPYAKLNFWDVDLKDRFTLDLSQFPLGRKFLLQIGARRRSVVPSRKRRAPTPSPASTKRKRSKK

SEQ ID NO: 40 - protein, 505 aa, Bovine papillomavirus type 6
MSYWLPSSGKLFLPPPTPVSNILNTDDFVTRTDTFYHASSERLLNVGHPYFELKKGEEVIVPKVSGSQFRVFRLQLPDPNKFTFQTPNVYNPETQRLVWALKGIEICRGQPLGIGVTGHPSFNKFRDAENLNNNQPSQGEDDRVNTALDPKQVQLFIVGCTPCEGEHWDVAESCQPLEIGACPPLQLVNTLIQDGEMCDIGFGNINNKALQATKSDAPLDIVDQIVKYPDFLKMSSDLQGNSMFFYAKREQLYLRHLWARGGTVGEEIPPNGSPSPYYLPGKVKPLPSSVYFGGPSGSLVSSDQQIFNRPFWIQRAQGNNNGVCWHNQLFVTAVDSTRGTNFTISVPKKNMGVQPQDLYKSTDFNHYLRHVEEWELSCIMQLCIVDLKPETLAHLHNMDPRILETWNLGFIQPPTNIEDQYRFIKSLATKCPGKEETAEKEDPYAKYKFWDVNLTERFSSNLEQYSLGRKFLFQIGKRGSKRPAPKTVTFDSSSKKAPKRRRKNA